(19) United States
(12) Patent Application Publication
(10) Pub. No.: US 2020/0181153 A1
(43) Pub. Date: Jun. 11, 2020

(54) IMIDAZOPYRIDAZINONE COMPOUNDS AND USES THEREOF

(71) Applicant: Aduro Biotech, Inc., Berkeley, CA (US)

(72) Inventors: Chudi Obioma Ndubaku, Oakland, CA (US); Tucker Curran Roberts, Berkeley, CA (US); Thomas Johnson, Saint-Laurent, CA (US); Stephane Ciblat, Montreal, CA (US); Yeeman K. Ramtohul, Pierrefonds, CA (US); Bernadette K. Latimer, Berkeley, CA (US)

(21) Appl. No.: 16/708,850

(22) Filed: Dec. 10, 2019

Related U.S. Application Data
(60) Provisional application No. 62/777,703, filed on Dec. 10, 2018.
(60) Provisional application No. 62/807,404, filed on Feb. 19, 2019.

(51) Int. Cl. C07D 487/04

(52) U.S. Cl. CPC C07D 487/04; A61K 45/06

(57) ABSTRACT

The present invention relates to imidazopyridazinone compounds. The present invention also relates to pharmaceutical compositions containing these compounds and methods of treating autoimmune, inflammatory, and neurodegenerative diseases by administering these compounds and pharmaceutical compositions to subjects in need thereof. The present invention also relates to the use of such compounds for research or other non-therapeutic purposes.

### RELATED APPLICATIONS

This application claims priority to, and the benefit of, U.S. Provisional Application No. 62/777,703, filed on Dec. 10, 2018; and 62/807,404, filed on Feb. 19, 2019, the contents of each of which are incorporated herein by reference in their entirety.

### BACKGROUND

The enzyme cyclic GMP-AMP Synthase (cGAS) catalyzes the synthesis of cyclic GMP-AMP (cGAMP) from ATP and GTP in the presence of DNA. This cGAMP then functions as a second messenger that binds to and activates STimulator of INterferon Genes (STING). The activation of IRF3 and the NF-κB signaling by this pathway results in the production of cytokines and type I interferons, which triggers an innate immune response to bacterial or viral infection. Genetic mutations that alter the balance of this pathway may result in an increased activation of the STING pathway, resulting in autoimmune and inflammatory diseases. For example, a loss of function mutation of TREX1 exonuclease, which digests DNA, can result in an accumulation of self-DNA in the cytosol, leading to excessive levels of cGAMP produced by cGAS and elevated expression of interferon induced genes in this pathway. Mutations in TREX1 are associated with systemic inflammatory diseases such as Aicardi-Goutieres Syndrome, familial chilblain lupus and systemic lupus erythematosus. Trex^{-/-} mice were shown to exhibit autoimmune and inflammatory phenotypes which are eliminated with genetic deletion of cGas in these mice (Gao et al., PNAS 112(42):E5699-705, 2015; Gray et al., The Journal of Immunology 195:1939-1943, 2015). Thus there is a need for inhibitors of the cGAS/STING pathway for the treatment of a variety of diseases.

### SUMMARY

The present invention provides the compounds of Formula (I):

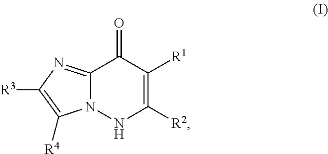

or a pharmaceutically acceptable salt, pharmaceutically acceptable solvate or pharmaceutically acceptable hydrate thereof. In this formula:

R¹ is Q¹-T¹-(X¹)ₙ;

Q¹ is a bond or C₁₋₃alkylene, wherein the C₁₋₃alkylene group is optionally substituted with one or more substituents independently selected from the group consisting of halo, C₁₋₆alkyl, C₂₋₆alkenyl, C₂₋₆alkynyl, cyano, C₁₋₆haloalkyl, —OR^{w2}, and —NR^{w2}R^{x2};

T¹ is H, halo, cyano, C₃₋₈cycloalkyl, C₆₋₁₀aryl, 3- to 12-membered heterocycloalkyl, 5- to 10-membered heteroaryl, —C(═O)C₀₋₃alkylene-C₃₋₈cycloalkyl, —C(═O)—C₀₋₃alkylene-C₆₋₁₀aryl, —C(═O)—C₀₋₃alkylene-3 to 12-membered heterocycloalkyl, —C(═O)—C₀₋₃alkylene-5 to 10-membered heteroaryl, —C(═O)R^{a}, —C(═O)OR^{a}, —NR^{a}R^{b}, —S(═O)₂R^{a}, —NR^{a}C(═O)R^{a}, —NR^{a}C(═O)NR^{a}R^{b}, —NR^{a}C(═O)OR^{a}, —NR^{a}S(═O)₂R^{a}, —C(═O)NR^{a}S(═O)₂R^{a}, —NR^{a}S(═O)₂NR^{a}R^{b}, —C(═O)NR^{a}R^{b}, or —S(═O)₂NR^{a}R^{b};

each X¹ is independently selected from the group consisting of halo, cyano, oxo, C₀₋₃alkylene-C(═O)R^{c}, C₀₋₃alkylene-OR^{c}, C₀₋₃alkylene-C(═O)OR^{c}, C₀₋₃alkylene-OC(═O)R^{c}, C₀₋₃alkylene-NR^{c}R^{d}, C₀₋₃alkylene-NR^{c}R^{d}R^{d′}, C₀₋₃alkylene-S(═O)ₘR^{c}, C₀₋₃alkylene-NR^{c}C(═O)R^{c}, C₀₋₃alkylene-NR^{c}C(═O)NR^{c}R^{d}, C₀₋₃alkylene-OC(═O)NR^{c}R^{d}, C₀₋₃alkylene-NR^{c}C(═O)OR^{c}, C₀₋₃alkylene-NR^{c}S(═O)₂R^{c}, C₀₋₃alkylene-C(═O)NR^{c}S(═O)₂R^{c}, C₀₋₃alkylene-NR^{c}S(═O)₂NR^{c}R^{d}, C₀₋₃alkylene-C(═O)NR^{c}R^{d}, C₀₋₃alkylene-S(═O)₂NR^{c}R^{d}, C₀₋₃alkylene-C(═NR^{c})NR^{c}R^{d}, C₀₋₃alkylene-NR^{c}C(═NR)NR^{c}R^{d}, and R^{S1}, in which R^{S1} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S1} is optionally substituted with one or more substituents independently selected from the group consisting of halo, cyano, nitro, oxo, C₁₋₆alkyl, C₂₋₆alkenyl, C₂₋₆alkynyl, C₁₋₆haloalkyl, C₀₋₃alkylene-NR^{e}R^{f}, C₀₋₃alkylene-OR^{e}, C₀₋₃alkylene-NR^{e}C(═O)R^{e}, C₀₋₃alkylene-NR^{e}C(═O)OR^{e}, C₀₋₃alkylene-NR^{e}C(═O)NR^{e}R^{f}, C₀₋₃alkylene-OC(═O)R^{e}, C₀₋₃alkylene-C(═O)OR^{e}, C₀₋₃alkylene-C(═O)NR^{e}R^{f}, C₀₋₃alkylene-C(═O)R^{e}, C₀₋₃alkylene-S(═O)ₘR^{e}, C₀₋₃alkylene-S(═O)₂NR^{e}R^{f}, C₀₋₃alkylene-NR^{e}S(═O)₂R^{e}, C₀₋₃alkylene-C(═O)NR^{e}S(═O)₂R^{e}, C₀₋₃alkylene-NR^{e}S(═O)₂NR^{e}R^{f}, and R^{S2}, in which R^{S2} is C₀₋₃alkylene-C₀₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl,

and each R^{S2} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w}, and —NR^{w}R^{x};

R² is Q²-T²-(X²)ₚ;

Q² is a bond or C₁₋₃alkylene, wherein the C₁₋₃alkylene group is optionally substituted with one or more substituents independently selected from the group consisting of halo, C₁₋₆alkyl, C₂₋₆alkenyl, C₂₋₆alkynyl, cyano, C₁₋₆haloalkyl, —OR^{w3}, and —NR^{w3}R^{x3};

T² is H, halo, cyano, C₁₋₆alkyl, C₂₋₆alkenyl, C₂₋₆alkynyl, C₃₋₈cycloalkyl, C₆₋₁₀aryl, 3- to 12-membered heterocycloalkyl, 5- to 10-membered heteroaryl, —C(═O)—C₀₋₃alkylene-C₃₋₈cycloalkyl, —C(═O)—C₀₋₃alkylene-C₆₋₁₀aryl, —C(═O)—C₀₋₃alkylene-3- to 12-membered heterocycloalkyl, —C(═O)—C₀₋₃alkylene-5- to 10-membered heteroaryl, —OR^{z}, —S(═O)ₘR^{k}, —P(═O)R^{kk}R^{mm}, —NR^{k}R^{m}, —C(═O)OR^{k}, or —C(═O)NR^{k}R^{m};

each X² is independently selected from the group consisting of halo, cyano, oxo, C₀₋₃alkylene-ORⁿ, C₀₋₃alkylene-S(═O)ₘRⁿ, C₀₋₃alkylene-NRⁿR^{o}, C₀₋₃alkylene-C(═O)NRⁿR^{o}, C₀₋₃alkylene-C(═O)ORⁿ, and R^{S3}, in which R^{S3} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and R^{S3} is optionally substituted with one or more substituents independently selected from the group consisting of halo, oxo, cyano, C₀₋₃alkylene-OR^{p}, C₀₋₃alkylene-S(═O)ₘR^{p}, C₀₋₃alkylene-NR^{p}R^{q}, C₀₋₃alkylene-C(═O)NR^{p}R^{q}, C₀₋₃alkylene-C₀₋₃alkylene-C(═O)OR^{p}, C₁₋₆alkyl, C₂₋₆alkenyl, C₂₋₆alkynyl, C₁₋₆haloalkyl, and R^{S4}, in which R^{S4} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S4} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w4}, and —NR^{w4}R^{x4};

R³ is halo, C₁₋₃alkyl, C₁₋₃haloalkyl, C₂₋₃alkenyl, C₂₋₃alkynyl, C₃₋₆cycloalkyl, —CN, —OR^{r}, —C(═O)R^{r}, —S(═O)ₘR^{r}, NR^{r}R^{t}, or —C(═O)OR^{r}, wherein C₁₋₃alkyl, C₂₋₃alkenyl and C₂₋₃alkynyl are optionally substituted with one C₃₋₆cycloalkyl;

R⁴ is C₁₋₆alkyl, C₁₋₆haloalkyl, S(═O)ₘR^{u}, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl, wherein C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl are optionally substituted with one or more substituents independently selected from the group consisting of halo, oxo, C₁₋₆alkyl, C₂₋₆alkenyl, C₂₋₆alkynyl, cyano, C₁₋₆haloalkyl, OR^{w5}, and NR^{w5}R^{x5};

each of R^{a} and R^{b}, independently, is H or R^{S5}, in which R^{S5} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and R^{S5} is optionally substituted with one or more substituents independently selected from the group consisting of halo, cyano, oxo, C₁₋₆haloalkyl, C₀₋₃alkylene-OR^{c2}, C₀₋₃alkylene-C(═O)R^{c2}, C₀₋₃alkylene-C(═O)OR^{c2}, C₀₋₃alkylene-OC(═O)R^{c2}, C₀₋₃alkylene-C(═O)NR^{c2}R^{d2}, C₀₋₃alkylene-S(═O)ₘR^{c2}, C₀₋₃alkylene-S(═O)₂NR^{c2}R^{d2}, C₀₋₃alkylene-NR^{c2}R^{d2}, C₀₋₃alkylene-NR^{c2}C(═O)R^{c2}, C₀₋₃alkylene-NR^{c2}C(═O)OR², C₀₋₃alkylene-NR^{c2}C(═O)NR^{c2}R^{d2}, C₀₋₃alkylene-NR^{c2}S(═O)₂R^{c2}, C₀₋₃alkylene-C(═O)NR²S(═O)₂R², C₀₋₃alkylene-NR^{c2}S(═O)₂NR^{c2}R^{d2}, C₀₋₃alkylene-N(S(═O)₂R^{c2})₂, and R^{S6}, in which R^{S6} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S6} is optionally substituted with one or more substituents independently selected from the group consisting of halo, cyano, oxo, C₁₋₆alkyl, C₂₋₆alkenyl, C₂₋₆alkynyl, C₁₋₆haloalkyl, C₀₋₃alkylene-NR^{e2}R², C₀₋₃alkylene-OR^{e2}, C₀₋₃alkylene-NR^{e2}C(═O)R^{e2}, C₀₋₃alkylene-NR^{e2}C(═O)OR^{e2}, C₀₋₃alkylene-NR^{e2}C(═O)NR^{e}R^{f2}, C₀₋₃alkylene-OC(═O)R^{e2}, C₀₋₃alkylene-C(O)OR^{e2}, C₀₋₃alkylene-C(═O)NR^{e2}R^{f2}, C₀₋₃alkylene-C(═O)R^{e2}, C₀₋₃alkylene-S(═O)ₘR^{e2}, C₀₋₃alkylene-S(═O)₂NR^{e2}R², C₀₋₃alkylene-NR²S(═O)₂R^{e2}, C₀₋₃alkylene-C(═O)NR^{e2}S(═O)₂R^{e2}, C₀₋₃alkylene-NR^{e2}S(═O)₂NR^{e2}R^{f2}, and R^{S7}, in which R^{S7} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S7} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w6}, and —NR^{w6}R^{x6};

each of R^{c}, R^{c2}, R^{d}, R^{d′}, and R^{d2}, independently, is H or R^{S8}, in which R^{S8} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S8} is optionally substituted with one or more substituents independently selected from the group consisting of halo, cyano, oxo, C₁₋₆alkyl, C₂₋₆alkenyl, C₂₋₆alkynyl, C₁₋₆haloalkyl, C₀₋₃alkylene-NR^{e3}R^{f3}, C₀₋₃alkylene-OR^{e3}, C₀₋₃alkylene-C(═O)OR^{e3}, C₀₋₃alkylene-C(═O)NR^{e3}R^{f3}, C₀₋₃alkylene-C(═O)R^{e3}, C₀₋₃alkylene-S(═O)ₘR^{e3}, C₀₋₃alkylene-S(═O)₂NR^{e3}R^{f3}, C₀₋₃alkylene-NR^{f3}C(═O)R^{e3}, C₀₋₃alkylene-NR^{f3}S(═O)ₘR^{e3}, and R^{S9}, in which R^{S9} is C₀₋₃alkylene-C₃₋₈cycloalkyl, C₀₋₃alkylene-C₆₋₁₀aryl, C₀₋₃alkylene-3- to 12-membered heterocycloalkyl, C₀₋₃alkylene-5- to 10-membered heteroaryl;

and each R^{S9} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w7}, and —NR^{w7}R^{x7};

each of R^{e}, R^{e2}, R^{e3}, R^{f}, R^{f2}, and R^{f3}, independently, is H or R^{S10}, in which R^{S10} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S10} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w8}, and —NR^{w8}R^{x8};

each of R^{kk} and R^{mm}, is independently selected from the group consisting of R^{k}, —OR^{k}, and —NR^{k}R^{m};

each of R^{k} and R^{m}, independently, is H or R^{z}, in which R^{z} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R⁷ is optionally substituted with one or more substituents independently selected from the group consisting of halo, cyano, oxo, C₁₋₆alkyl, C₂₋₆alkenyl, C₂₋₆alkynyl, C₁₋₆haloalkyl, C₀₋₃alkylene-NRⁿ²R^{o2}, C₀₋₃alkylene-ORⁿ², C₀₋₃alkylene-C(═O)ORⁿ², C₀₋₃alkylene-C(═O)NRⁿ²R^{o2}, C₀₋₃alkylene-C(═O)Rⁿ², C₀₋₃alkylene-S(═O)ₘRⁿ², C₀₋₃alkylene-S(═O)₂NRⁿ²R^{o2}, and R^{S11}, in which R^{S11} is C₁₋₃alkylene-C₃₋₈cycloalkyl, C₀₋₃alkylene-C₆₋₁₀aryl, C₀₋₃alkylene-3- to 12-membered heterocycloalkyl, C₀₋₃alkylene-5- to 10-membered heteroaryl;

and each R^{S11} is optionally substituted with one or more substituents independently selected from the group consisting of halo, oxo, cyano, C₀₋₃alkylene-OR², C₀₋₃alkylene-S(═O)ₘR², C₀₋₃alkylene-NR^{p2}R^{q2}, C₀₋₃alkylene-C(═O)NR^{p2}R^{q2}, C₀₋₃alkylene-C₀₋₃alkylene-C(═O)OR^{p2}, C₁₋₆alkyl, C₂₋₆alkenyl, C₂₋₆alkynyl, C₁₋₆haloalkyl, and R^{S12}, in which R^{S12} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each R^{S12} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w9}, and —NR^{w9}R^{x9};

each of Rⁿ, Rⁿ², R^{o}, and R^{o2}, independently, is H or R^{S13}, in which R^{S13} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each R^{S13} is optionally substituted with one or more substituents independently selected from the group consisting of halo, oxo, cyano, C₀₋₃alkylene-OR^{p3}, C₀₋₃alkylene-S(═O)ₘR^{p3}, C₀₋₃alkylene-NR^{p3}R^{q3}, C₀₋₃alkylene-C(═O)NR^{p3}R^{q3}, C₀₋₃alkylene-C(═O)OR^{p3}, C₁₋₆alkyl, C₂₋₆alkenyl, C₂₋₆alkynyl, C₁₋₆haloalkyl, and R^{S14}, in which R^{S14} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each R^{S14} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w10}, and —NR^{w10}R^{x10};

each of R^{p}, R^{p2}, R^{p3}, R^{q}, R^{q2}, and R^{q3}, independently, is H or R^{S15}, in which R^{S15} is C₁₋₆alkyl, C₂₋₆alkenyl, C₂₋₆alkynyl, C₀₋₃alkylene-C-cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each R^{S51} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w11}, and —NR^{w11}R^{x11};

each of R^{r}, R^{t}, and R^{u}, independently, is H or R^{S16}, in which R^{S16} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S16} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —C(═O)OR^{w12}, —OR^{w12}, and —NR^{w12}R^{x12};

each R^{w}, R^{w2}, R^{w3}, R^{w4}, R^{w5}, R^{w6}, R^{w7}, R^{w8}, R^{w9}, R^{w10}, R^{w11}, R^{w12}, R^{x}, R^{x2}, R^{x3}, R^{x4}, R^{x5}, R^{x6}, R^{x7}, R^{x8}, R^{x9}, R^{x10}, R^{x11}, and R^{x12}, independently, is H, C₁₋₆alkyl, C₂₋₆alkenyl, C₂₋₆alkynyl or C₁₋₆haloalkyl;

each of n and p independently is 0, 1, 2, 3, 4, or 5, wherein when T¹ is H, n is 0, and when T² is H, p is 0; and

m is 0, 1, or 2;

with the proviso that the compound is not

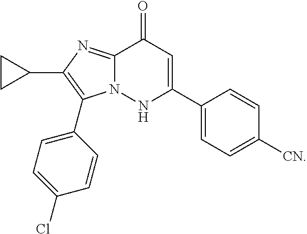 [SMILES: N#Cc1ccc(-c2cc(=O)c3nc(C4CC4)c(-c4ccc(Cl)cc4)n3[nH]2)cc1]

For example, Q¹ is a bond or —CH₂— and T¹ is C₃₋₈cycloalkyl, C₆₋₁₀aryl, 3- to 12-membered heterocycloalkyl, 5- to 10-membered heteroaryl, or —C(═O)NR^{a}R^{b}.

For example, Q¹ is a bond and T¹ is C₃₋₈cycloalkyl, C₆₋₁₀aryl, 3- to 12-membered heterocycloalkyl, or 5- to 10-membered heteroaryl.

For example, Q¹ is a bond and T¹ is C₆₋₁₀aryl, 3- to 12-membered heterocycloalkyl, or 5- to 10-membered heteroaryl.

For example Q¹ is a bond and T¹ is phenyl, 5- or 6-membered monocyclic heteroaryl, or 9- or 10-membered bicyclic heteroaryl, preferably wherein T¹ is 9- or 10-membered bicyclic heteroaryl.

For example, Q¹ is a bond or —CH₂—, T¹ is —C(═O)NR^{a}R^{b} and n is 0.

For example, one of R^{a} and R^{b} is H or methyl and the other of R^{a} and R^{b} is not H or methyl.

For example, R² is Q¹-T²-(X²)ₚ. Q² is a bond, T² is H, halo, cyano, C₁₋₆alkyl, C₁₋₆haloalkyl, C₂₋₆alkenyl, C₂₋₆alkynyl, C₃₋₈cycloalkyl, C₆₋₁₀aryl, 3- to 12-membered heterocycloalkyl, or 5- to 10-membered heteroaryl, each X² independently is halo, cyano, oxo, C₀₋₃alkylene-ORⁿ, C₀₋₃alkylene-S(═O)ₘRⁿ, C₀₋₃alkylene-NRⁿR^{o}, C₀₋₃alkylene-C(═O)NRⁿR^{o}, C₀₋₃alkylene-C₀₋₃alkylene-C(═O)ORⁿ, and each Rⁿ and R^{o} is independently H, C₁₋₆alkyl, C₂₋₆alkenyl, C₂₋₆alkynyl or C₁₋₆haloalkyl.

For example, R² is Q²-T²-(X²)ₚ, Q² is a bond, T² is H, halo, cyano, C₁₋₆alkyl, C₁₋₆haloalkyl, C₂₋₆alkenyl, or C₂₋₆alkynyl, and each X² independently is halo or OC₁₋₆alkyl.

For example, R² is H, cyano, methyl or methoxymethyl.

For example, R² is H, methyl or methoxymethyl.

For example, R³ is C₁₋₃alkyl, C₁₋₃haloalkyl, —CN, —S(═O)₂C₁₋₃alkyl or —C(═O)OC₁₋₃alkyl.

For example, R³ is —CN, C₁₋₃alkyl, C₁₋₃haloalkyl or —C(═O)OC₁₋₃alkyl.

For example, R³ is C₁₋₃alkyl, C₁₋₃haloalkyl or —C(═O)OC₁₋₃alkyl.

For example, R³ is —CF₃, methyl or —C(═O)OC₁₋₃alkyl.

For example, R³ is —CF₃ or —CN.

For example, R³ is —CF₃.

For example, R³ is —CN.

For example, R⁴ is C₁₋₃alkyl, C₁₋₃haloalkyl, —S(═O)₂C₁₋₃alkyl, C₃₋₈cycloalkyl, C₆₋₁₀aryl, 3- to 12-membered heterocycloalkyl, or 5- to 10-membered heteroaryl, wherein C₃₋₈cycloalkyl, C₆₋₁₀aryl, 3- to 12-membered heterocycloalkyl, or 5- to 10-membered heteroaryl are optionally substituted with 1-3 substituents selected from the group consisting of halo, oxo, C₁₋₆alkyl, C₂₋₆alkenyl, C₂₋₆alkynyl, cyano, C₂₋₆haloalkyl, —OR^{w5}, and —NR^{w5}R^{x5}.

For example, R⁴ is C₃₋₈cycloalkyl, C₆₋₁₀aryl, 3 to 12-membered heterocycloalkyl, or 5- to 10-membered heteroaryl, wherein C₃₋₈cycloalkyl, C₆₋₁₀aryl, 3- to 12-membered heterocycloalkyl, or 5- to 10-membered heteroaryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

For example, R⁴ is C₃₋₈cycloalkyl or C₆₋₁₀aryl, wherein C₃₋₈cycloalkyl and C₆₋₁₀aryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

For example, R⁴ is phenyl optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

For example, R⁴ is C₃₋₈cycloalkyl.

For example, R⁴ is cyclopentyl.

For example, R⁴ is phenyl.

For example, the compound can be of Formula (Ia):

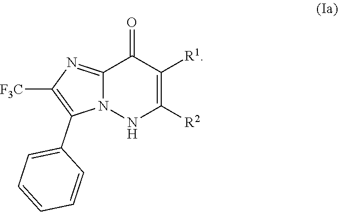

For example, Q¹ is a bond or —CH₂— and T¹ is C₃₋₈cycloalkyl, C₆₋₁₀aryl, 3- to 12-membered heterocycloalkyl, 5- to 10-membered heteroaryl, or —C(═O)NR^{a}R^{b}.

For example, Q¹ is a bond and T¹ is C₃₋₈cycloalkyl, C₆₋₁₀aryl, 3- to 12-membered heterocycloalkyl, or 5- to 10-membered heteroaryl.

For example, Q¹ is a bond and T¹ is C₆₋₁₀aryl, 3- to 12-membered heterocycloalkyl, or 5- to 10-membered heteroaryl.

For example Q¹ is a bond and T¹ is phenyl, 5- or 6-membered monocyclic heteroaryl, or 9- or 10-membered bicyclic heteroaryl, preferably wherein T¹ is 9- or 10-membered bicyclic heteroaryl.

For example, Q¹ is a bond or —CH₂—, T¹ is —C(═O)NR^{a}R^{b} and n is 0.

For example, one of R^{a} and R^{b} is H or methyl and the other of R^{a} and R^{b} is not H or methyl.

For example, n is 0.

For example, T¹ is aryl or heteroaryl, preferably phenyl, 5- or 6-membered monocyclic heteroaryl, or 9- or 10-membered bicyclic heteroaryl.

For example, T¹ is 5- to 10-membered heteroaryl.

For example, T¹ is pyridinyl, pyrazinyl, pyridazinyl, pyrimidinyl, indolyl, indolinyl, isoindolyl, isoindolinyl, indazolyl, pyrazolopyridinyl, pyrazolopyrimidinyl, oxazolopyrimidinyl, oxazolopyridinyl, imidazopyridinyl, benzimidazolyl, tetrahydrobenzimidazolyl, benzofuranyl, dihydrobenzofuranyl, isobenzofuranyl, dihydroisobenzofuranyl, triazolopyridinyl, benzothiazolyl, azabenzimidazolyl, azabenzoxazolyl, azabenzothiazolyl, imidazopyridinyl, quinolinyl, isoquinolinyl, quinazolinyl, oxazolyl, isoxazolyl, thiazolyl, isothiazolyl, benzoxazolyl, benzodioxolyl, chromanyl, tetrahydrooxazoloazepinyl, tetrahydrobenzoxazolyl, oxadiazolyl, thiadiazolyl, pyrazolyl, triazolyl, imidazolyl, furanyl, or thiophenyl.

For example, T¹ is pyridinyl, pyrazinyl, pyridazinyl, pyrimidinyl, indolyl, indazolyl, pyrazolopyridinyl, benzimidazolyl, benzothiazolyl, azabenzimidazolyl, azabenzoxazolyl, azabenzothiazolyl, imidazopyridinyl, quinolinyl, isoquinolinyl, quinazolinyl, oxazolyl, isoxazolyl, thiazolyl, isothiazolyl, benzoxazolyl, oxadiazolyl, thiadiazolyl, triazolyl, imidazolyl, furanyl, or thiophenyl.

For example, T¹ is C₀₋₁alkylene-C₆₋₁₀aryl.

For example, T¹ is phenyl, benzyl, naphthyl, or CH₂naphthyl.

For example, T¹ is 3- to 12-membered heterocycloalkyl, preferably 4- to 10-membered heterocycloalkyl.

For example, T¹ is piperazine, piperidine, quinuclidine, or morpholine.

Subsets of compounds of Formula (I) includes those of Formula (IIa) or Formula

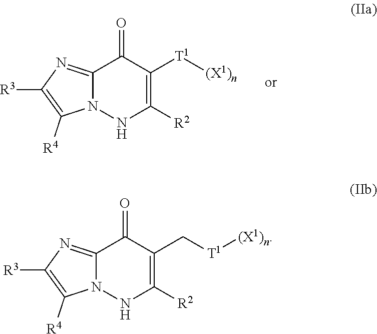

For example, T¹ is C₃₋₈cycloalkyl, C₆₋₁₀aryl, 3- to 12-membered heterocycloalkyl, 5- to 10-membered heteroaryl, —C(═O)—C₀₋₃alkylene-C₃₋₈cycloalkyl, —C(═O)—C₀₋₃alkylene-C₆₋₁₀aryl, —C(═O)—C₀₋₃alkylene-3- to 12-membered heterocycloalkyl, —C(═O)—C₀₋₃alkylene-5- to 10-membered heteroaryl, —NR^{a}R^{b}, —S(═O)₂R^{a}, —NR^{a}C(═O)R^{a}, —NR^{a}C(═O)NR^{a}R^{b}, —NR^{a}C(═O)OR^{a}, —NR^{a}S(═O)₂R^{a}, —C(═O)NR^{a}S(═O)₂R^{a}, —NR^{a}S(═O)₂NR^{a}R^{b}, —C(═O)NR^{a}R^{b}, or —S(═O)₂NR^{a}R^{b}; each X¹ independently is halo, cyano, oxo, C₀₋₃alkylene-C(═O)R^{c}, C₀₋₃alkylene-OR^{c}, C₀₋₃alkylene-NR^{c}R^{d}, C₀₋₃alkylene-OC(═O)NR^{c}R^{d}, C₀₋₃alkylene-C(═NR^{c})NR^{c}R^{d}, C₀₋₃alkylene-NR^{c}C(═NR^{c})NR^{c}R^{d}, or R^{S1}, in which R^{S1} is C₁₋₆alkyl, C₂₋₆alkenyl, C₂₋₆alkynyl, C₁₋₆haloalkyl, C₀₋₃alkylene-C₃₋₈cycloalkyl, C₀₋₃alkylene-C₆₋₁₀aryl, C₀₋₃alkylene-3 to 12-membered heterocycloalkyl, or C₀₋₃alkylene-5 to 10-membered heteroaryl, and R^{S1} is optionally substituted with one or more substituents independently selected from the group consisting of halo, oxo, C₁₋₆alkyl, C₂₋₆alkenyl, C₂₋₆alkynyl, C₁₋₆haloalkyl, C₀₋₃alkylene-NR^{e}R^{f}, C₀₋₃alkylene-OR^{e}, C₀₋₃alkylene-NR^{e}C(═O)R^{e}, C₀₋₃alkylene-NR^{e}C(═O)OR^{e}, C₀₋₃alkylene-NR^{e}C(═O)NR^{e}R^{f}, C₀₋₃alkylene-OC(═O)R^{e}, C₀₋₃alkylene-C(═O)OR^{e}, C₀₋₃alkylene-C(═O)R^{e}, C₀₋₃alkylene-S(═O)ₘR, C₀₋₃alkylene-S(═O)₂NR^{e}R^{f}, C₀₋₃alkylene-NR^{e}S(═O)₂R^{e}, C₀₋₃alkylene-NR^{e}S(═O)₂NR^{e}R^{f}, and R^{S2}, in which R^{S2} is C₀₋₃alkylene-C₃₋₈cycloalkyl, C₀₋₃alkylene-C₆₋₁₀aryl, C₀₋₃alkylene-3- to 12-membered heterocycloalkyl, C₀₋₃alkylene-5- to 10-membered heteroaryl, and R^{S2} is optionally substituted with one or more substituents independently selected from the group consisting of halo, C₁₋₆alkyl, C₂₋₆alkenyl, C₂₋₆alkynyl, cyano, C₁₋₆haloalkyl, —OR^{w}, and —NR^{w}R^{x}.

Subsets of compounds of Formula (I) includes those of Formula (IIc) or Formula (IId):

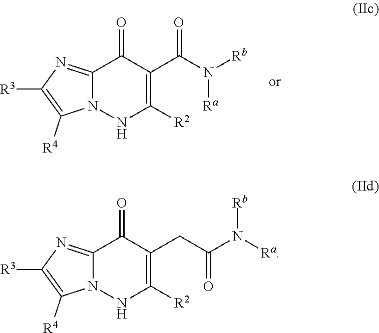

Subsets of compounds of Formula (I) includes those of Formula (IIe) or Formula (IIf):

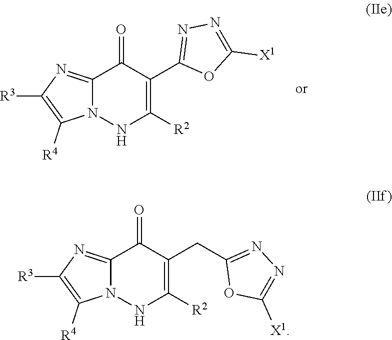

For example, one of R^{a} and R^{b} independently is 5 to 10-membered heteroaryl and the other is hydrogen.

For example, one of R^{a} and R^{b} independently is pyridinyl, pyrazinyl, pyridazinyl, pyrimidinyl, indolyl, indazolyl, benzimidazolyl, imidazopyridinyl, quinolinyl, isoquinolinyl, quinazolinyl, oxazolyl, isoxazolyl, thiazolyl, isothiazolyl, benzoxazolyl, oxadiazolyl, triazolyl, imidazolyl, furan, or thiophenyl, and the other of R^{a} and R^{b} is hydrogen.

For example, one of R^{a} and R^{b} is C₀₋₁alkylene-C₆₋₁₀aryl.

For example, one of R^{a} and R^{b} independently is phenyl, benzyl, naphthyl, or CH₂naphthyl.

For example, one of R^{a} and R^{b} independently is 5- to 9-membered heterocycloalkyl.

For example, one of R^{a} and R^{b} independently is dihydrobenzofuran, tetrahydrobenzimidazole, morpholine, tetrahydrofuran, piperidine, or piperazine.

For example, each of R^{a} and R^{b} independently is C₅₋₆cycloalkyl.

For example, each of R^{a} and R^{b} independently is cyclohexane or cyclopropane.

For example, X¹ is C₀₋₁alkylene-C₆₋₁₀aryl.

For example, X¹ is phenyl, benzyl, naphthyl, or CH₂naphthyl.

For example, X¹ is 5- to 10-membered heteroaryl.

For example, X¹ is benzoxazolyl, benzimidazolyl, pyridinyl, pyrazinyl, pyridazinyl, pyrimidinyl, indolyl, indazolyl, imidazopyridinyl, quinolinyl, isoquinolinyl, quinazolinyl, oxazolyl, isoxazolyl, thiazolyl, isothiazolyl, benzoxazolyl, oxadiazolyl, triazolyl, imidazolyl, furanyl, or thiophenyl.

For example, X¹ is 5- to 9-membered heterocycloalkyl.

For example, X¹ is tetrahydrobenzoxazole, tetrahydrobenzimidazole, morpholine, tetrahydrofuran, tetrahydropyran, piperidine, pyrrolidine, or piperazine.

For example, X¹ is C₃₋₆cycloalkyl.

For example, X¹ is OR^{a} or C(═O)C₁₋₆alkyl.

For example, X¹ is C₁₋₃alkyl.

For example, X¹ is OCF₃, OC₁₋₃alkyl, NH₂, CN, OH or halo.

For example, X¹ is C₀₋₁alkylene-C(═NR^{c})NR^{c}R^{d}.

For example, X¹ is C₀₋₁alkylene-NR^{c}C(═NR^{c})NR^{c}R^{d}.

In one example, for a compound of Formula I, or a pharmaceutically acceptable salt, pharmaceutically acceptable solvate or pharmaceutically acceptable hydrate thereof;

R¹ is Q¹-T¹-(X¹)ₙ;

Q¹ is a bond, —CH₂—, or —CH₂CH₂—;

T¹ is C₆₋₁₀aryl, 3- to 12-membered heterocycloalkyl, 5- to 10-membered heteroaryl, —C(═O)—C₀₋₃alkylene-3- to 12-membered heterocycloalkyl, —NR^{a}R^{b}, —NR^{a}C(═O)R^{a}, —C(═O)NR^{a}S(═O)₂R^{a}, or —C(═O)NR^{a}R^{b};

each X¹ independently is halo, cyano, oxo, C₀₋₃alkylene-OR^{c}, C₀₋₃alkylene-C(═O)OR^{c}, C₀₋₃alkylene-NR^{c}R^{d}, C₀₋₃alkylene-N⁺R^{c}R^{d}R^{d′}, C₀₋₃alkylene-S(═O)ₘR^{c}, C₀₋₃alkylene-NR^{c}C(═O)R^{c}, C₀₋₃alkylene-OC(═O)NR^{c}R^{d}, C₀₋₃alkylene-C(═O)NR^{c}R^{d}, C₀₋₃alkylene-C(═NR^{c})NR^{c}R^{d}, C₀₋₃alkylene-NR^{c}C(═NR^{c})NR^{c}R^{d}, or R^{S1}, in which R^{S1} is C₁₋₆alkyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl,

and each R^{S1} is optionally substituted with one or more substituents independently selected from the group consisting of halo, cyano, nitro, oxo, C₁₋₆alkyl, C₁₋₆haloalkyl, C₀₋₃alkylene-NR^{e}R^{f}, C₀₋₃alkylene-OR^{e}, C₀₋₃alkylene-NR^{e}C(═O)R^{e}, C₀₋₃alkylene-C(═O)OR^{e}, C₀₋₃alkylene-C(═O)R, C₀₋₃alkylene-S(═O)ₘR^{o}, C₀₋₃alkylene-NR^{c}S(═O)₂R, and R^{S2}, in which R^{S2} is C₀₋₃alkylene-C₆₋₁₀aryl or C₀₋₃alkylene-3- to 12-membered heterocycloalkyl, and each R^{S2} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w}, and —NR^{w}R^{x};

each of R^{a} and R^{b}, independently, is H or R^{S5}, in which R^{S5} is C₁₋₆alkyl, C₀₋₃alkylene-C₃₋₈cycloalkyl, C₀₋₃alkylene-C₆₋₁₀aryl, C₀₋₃alkylene-3 to 12-membered heterocycloalkyl, or C₀₋₃alkylene-5- to 10-membered heteroaryl,

and R^{S5} is optionally substituted with one or more substituents independently selected from the group consisting of halo, cyano, oxo, C₁₋₆haloalkyl, C₀₋₃alkylene-OR^{c2}, C₀₋₃alkylene-C(═O)R^{c2}, C₀₋₃alkylene-C(═O)OR^{c2}, C₀₋₃alkylene-S(═O)ₘR^{c2}, C₀₋₃alkylene-NR^{c2}R^{d2}, C₀₋₃alkylene-NR^{c2}C(═O)R^{c2}, C₀₋₃alkylene-NR²C(═O)OR^{c2}, C₀₋₃alkylene-NR^{c2}S(═O)₂R^{c2}, C₀₋₃alkylene-N(S(═O)₂R^{c2})₂, and R^{S6}, in which R^{S6} is C₁₋₆alkyl, C₀₋₃alkylene-C₃₋₈cycloalkyl, or C₀₋₃alkylene-3- to 12-membered heterocycloalkyl,

and each R^{S6} is optionally substituted with one or more substituents independently selected from the group consisting of C₀₋₃alkylene-C₃₋₈cycloalkyl C₀₋₃alkylene-NR^{e2}R², C₀₋₃alkylene-OR^{e2};

R² is Q²-T²-(X²)ₚ;

Q² is a bond, —CH₂—, or —CH₂CH₂—;

T² is H, halo, cyano, C₁₋₆alkyl, C₃₋₈cycloalkyl, 3- to 12-membered heterocycloalkyl, 5- to 10-membered heteroaryl, C(═O)-3 to 12-membered heterocycloalkyl, —OR, —S(═O)ₘR^{k}, —P(═O)R^{kk}R^{mm}, —NR^{k}R^{m}, —C(═O)OR^{k} or —C(═O)NR^{k}R^{m};

each X² independently is halo, cyano, oxo, C₀₋₃alkylene-ORⁿ, C₀₋₃alkylene-C(═O)NRⁿR^{o}, C₀₋₃alkylene-C(═O)ORⁿ or R^{S3}, in which R^{S3} is C₁₋₆alkyl optionally substituted with C₀₋₃alkylene-OR^{p};

each of R^{kk}, and R^{mm}, is independently selected from the group consisting of R^{k}, —OR, and —NR^{k}R^{m};

each of R^{k}, and R^{m}, independently, is H or R^{z}, in which R^{z} is C₁₋₆alkyl, C₀₋₃alkylene-C₃₋₈cycloalkyl, C₀₋₃alkylene-3 to 12-membered heterocycloalkyl, or C₀₋₃alkylene-5 to 10-membered heteroaryl;

and each R^{z} is optionally substituted with one or more substituents independently selected from the group consisting of halo, C₀₋₃alkylene-NRⁿ²R^{o2}, C₀₋₃alkylene-ORⁿ², C₀₋₃alkylene-C(═O)ORⁿ², C₀₋₃alkylene-C(═O)NRⁿ²R^{o2}, and R^{S11}, in which R^{S11} is C₀₋₃alkylene-3 to 12-membered heterocycloalkyl,

and each R^{S11} is optionally substituted with one or more substituents independently selected from the group consisting of halo, oxo, cyano, C₀₋₃alkylene-OR^{p}—, C₀₋₃alkylene-S(═O)ₘR^{p2}, C₀₋₃alkylene-NR^{p2}R^{q2}, C₀₋₃alkylene-C(═O)NR^{p2}R^{q2}, C₀₋₃alkylene-C₀₋₃alkylene-C(═O)OR^{p2}, C₁₋₆alkyl, C₂₋₆alkenyl, C₂₋₆alkynyl, and C₁₋₆haloalkyl;

R³ is C₁₋₃alkyl, C₁₋₃haloalkyl, C₂₋₃alkenyl, C₂₋₃alkynyl, C₃₋₆cycloalkyl, —CN, —OR^{r}, —C(═O)R^{r}, —S(═O)ₘR^{r}, —NR^{r}R^{t}, or —C(═O)OR^{r}, wherein C₁₋₃alkyl, C₂₋₃alkenyl and C₂₋₃alkynyl are optionally substituted with C₃₋₆cycloalkyl;

R⁴ is C₁₋₃alkyl, C₁₋₃haloalkyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl, wherein C₀₋₃alkylene-C₃₋₈cycloalkyl, C₀₋₃alkylene-C₆₋₁₀aryl, C₀₋₃alkylene-3 to 12-membered heterocycloalkyl, or C₀₋₃alkylene-5 to 10-membered heteroaryl are optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5};

each of R^{c}, R^{c2}, R^{d}, R^{d′}, and R^{d2}, independently, is H, C₁₋₆alkyl, C₁₋₆haloalkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each of R^{e}, R^{e2}, R^{f}, and R^{f2}, independently, is H, C₁₋₆alkyl, C₁₋₆haloalkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3 to 12-membered heterocycloalkyl, or C₀₋₃alkylene-5- to 10-membered heteroaryl;

each of Rⁿ, Rⁿ², R^{o}, and R^{o2}, independently, is H or R^{S13}, in which R^{S13} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl; and each R^{S13} is optionally substituted with one or more substituents independently selected from the group consisting of halo, oxo, cyano, C₀₋₃alkylene-OR^{p3}, C₀₋₃alkylene-S(═O)ₘR^{p3}, C₀₋₃alkylene-NR^{p3}R^{q3}, C₀₋₃alkylene-C(═O)NR^{p3}R^{q3}, C₀₋₃alkylene-C(═O)OR^{p3}, C₁₋₆alkyl, C₂₋₆alkenyl, C₂₋₆alkynyl, C₁₋₆haloalkyl, C₀₋₃alkylene-C₃₋₈cycloalkyl, C₀₋₃alkylene-C₆₋₁₀aryl, C₀₋₃alkylene-3- to 12-membered heterocycloalkyl, and C₀₋₃alkylene-5- to 10-membered heteroaryl;

each of R^{p}, R^{p2}, R^{p3}, R^{q2}, and R^{q3}, independently, is H, C₁₋₆alkyl, C₁₋₆haloalkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each of R^{r} and R^{t}, independently, is H, C₁₋₆alkyl, C₁₋₆haloalkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each R^{w}, R^{w5}, R^{x}, and R^{x5}, independently, is H, C₁₋₆alkyl, C₂₋₆alkenyl, C₂₋₆alkynyl or C₁₋₆haloalkyl;

each of n and p independently is 0, 1, 2, 3, 4, or 5, wherein when T¹ is H, n is 0, and when T² is H, p is 0; and

m is 0, 1, or 2.

In one example, for a compound of Formula I, or a pharmaceutically acceptable salt, pharmaceutically acceptable solvate or pharmaceutically acceptable hydrate thereof;

R¹ is —(CH₂)₀₋₁—C(═O)NR^{a}R^{b}; —CH₂CH₂—NR^{a}R^{b}; —CH₂CH₂—NR^{a}C(═O)R^{a}; —C(═O)NR^{a}S(═O)₂R^{a}; —(CH₂)₀₋₁—C₆₋₁₀aryl; —(CH₂)₀₋₁-5- to 6-membered monocyclic heteroaryl; —(CH₂)₀₋₁-9- to 10-membered bicyclic heteroaryl; a 4- to 6-membered monocyclic heterocycloalkyl; a 9- to 10-membered bicyclic heterocycloalkyl; —C(═O)-4- to 6-membered monocyclic heterocycloalkyl; —C(═O)-9- to 10-membered bicyclic heterocycloalkyl; wherein the aryl, 5heteroaryl, and heterocycloalkyl rings are optionally independently substituted with 1, 2, 3, 4, or 5 X¹;

each X¹ independently is halo; cyano; oxo; C₁₋₆alkyl optionally substituted with one or more substituents independently selected from the group consisting of halo, C₀₋₃alkylene-NR^{e}R^{f}, C₀₋₃alkylene-OR^{e}, C₀₋₃alkylene-C(═O)OR^{e}, C₀₋₃alkylene-C₃₋₆cycloalkyl, C₀₋₃alkylene-C₆₋₁₀aryl, and C₀₋₃alkylene-4 to 6-membered heterocycloalkyl, wherein heterocycloalkyl is optionally independently substituted with one or more C₁₋₆alkyl; C₀₋₃alkylene-C₃₋₄cycloalkyl optionally substituted with one or more substituents independently selected from the group consisting of C₁₋₆alkyl, C₁₋₆haloalkyl, C₁₋₃alkylene-NR^{e}R^{f}, and C₀₋₃alkylene-OR^{e}; C₀₋₃alkylene-C₆₋₁₀aryl, wherein C₆₋₁₀aryl is optionally substituted with one or more substituents selected from the group consisting of halo, cyano, nitro, C₁₋₆alkyl, C₁₋₆haloalkyl, C₀₋₃alkylene-NR^{e}R^{f}, C₀₋₃alkylene-OR^{e}, C₀₋₃alkylene-C(═O)OR^{e}, C₀₋₃alkylene-C(═O)R^{e}, C₀₋₃alkylene-S(═O)ₘR^{e}, and C₀₋₃alkylene-NR^{e}S(═O)₂R^{e}; C₀₋₃alkylene-4- to 6-membered monocyclic heterocycloalkyl or 9- or 10-membered bicyclic heterocycloalkyl, wherein heterocycloalkyl is optionally substituted with one or more substituents independently selected from the group consisting of oxo, C₀₋₃alkylene-NR^{e}R^{f}, and C₀₋₃alkylene-OR^{e}; C₀₋₃alkylene-5- or 6-membered monocyclic heteroaryl or 9- or 10-membered bicyclic heteroaryl, wherein heteroaryl is independently optionally substituted with one or more C₀₋₃alkylene-OR^{c}; C₀₋₃alkylene-OR^{c}; C₀₋₃alkylene-C(═O)OR^{c}; C₀₋₃alkylene-NR^{c}R^{d}; C₀₋₃alkylene-N⁺R^{c}R^{d}R^{d′}; C₀₋₃alkylene-S(═O)ₘR^{c}; C₀₋₃alkylene-NR^{c}C(═O)R^{c}; C₀₋₃alkylene-OC(═O)NR^{c}R^{d}; C₀₋₃alkylene-C(═O)NR^{c}R^{d}; C₀₋₃alkylene-C(═NR^{c})NR^{c}R^{d}; or C₀₋₃alkylene-NR^{c}C(═NR)NR^{c}R^{d};

each of R^{a} and R^{b}, independently, is H or R^{S5}, in which R^{S1} is C₁₋₆alkyl, C₀₋₃alkylene-C₃₋₆cycloalkyl, C₀₋₃alkylene-C₆₋₁₀aryl, C₀₋₃alkylene-4- to 6-membered monocyclic heterocycloalkyl, C₀₋₃alkylene-9- or 10-membered bicyclic heterocycloalkyl, C₀₋₃alkylene-5- or 6-membered monocyclic heteroaryl, or C₀₋₃alkylene-9- or 10-membered bicyclic heteroaryl;

and R^{S5} is optionally substituted with one or more substituents selected from the group consisting of halo, cyano, oxo, C₁₋₆haloalkyl, C₀₋₃alkylene-OR^{c2}, C₀₋₃alkylene-C(═O)R^{c2}, C₀₋₃alkylene-C(═O)OR², C₀₋₃alkylene-S(═O)ₘR, C₀₋₃alkylene-NR^{c2}R^{d2}, C₀₋₃alkylene-NR²C(═O)R², C₀₋₃alkylene-NR^{c2}C(═O)OR², C₀₋₃alkylene-NR^{c2}S(═O)₂R^{c}, C₀₋₃alkylene-N(S(═O)₂R^{c2})₂, and R^{S6}, in which R^{S6} is C₁₋₆alkyl, C₀₋₃alkylene-C₃₋₈cycloalkyl, or C₀₋₃alkylene-4- to 6-membered monocyclic heterocycloalkyl;

and each R^{S6} is optionally substituted with one or more C₀₋₃alkylene-C₃₋₆cycloalkyl, C₀₋₃alkylene-NR^{e2}R^{f}, C₀₋₃alkylene-OR^{e2};

R² is Q²-T²-(X²)ₚ;

Q² is a bond, —CH₂—, or —CH₂CH₂—:

T² is H, halo, cyano, C₁₋₆alkyl, C₃₋₆cycloalkyl, 4- to 6-membered monocyclic heterocycloalkyl, 9- or 10-membered bicyclic heterocycloalkyl, 5- or 6-membered monocyclic heteroaryl, 9- or 10-membered bicyclic heteroaryl, C(═O)-4- to 6-membered monocyclic heterocycloalkyl, —OR. —S(═O)ₘR^{k}, —P(═O)R^{k}R^{m}, —NR^{k}R^{m}, —C(═O)OR^{k} or —C(═O)NR^{k}R^{m};

each X² independently is halo, cyano, oxo, C₀₋₃alkylene-ORⁿ, C₀₋₃alkylene-C(═O)NRⁿR^{o}, C₀₋₃alkylene-C(═O)ORⁿ or C₁₋₆alkyl, wherein C₁₋₆alkyl is optionally substituted with one C₀₋₃alkylene-OR^{p};

each of R^{kk} and R^{mm} is independently selected from the group consisting of R^{k}, —OR^{k}, and —NR^{k}R^{m};

each of R^{k}, and R^{m}, independently, is H or R^{z}, in which R^{z} is C₁₋₆alkyl, C₀₋₃alkylene-C₃₋₆cycloalkyl, C₀₋₃alkylene-4- to 6-membered monocyclic heterocycloalkyl, C₀₋₃alkylene-9- or 10-membered bicyclic heterocycloalkyl, C₀₋₃alkylene-5 or 6-membered monocyclic heteroaryl, or C₀₋₃alkylene-9- or 10-membered bicyclic heteroaryl;

and each R^{z} is optionally substituted with one or more substituents selected from the group consisting of halo, C₁₋₆alkyl, C₀₋₃alkylene-NRⁿ²R², C₀₋₃alkylene-ORⁿ², C₀₋₃alkylene-C(═O)ORⁿ², C₀₋₃alkylene-C(═O)NRⁿ²R^{o2}, and R^{S1}, in which R^{S1} is C₀₋₃alkylene-4- to 6-membered monocyclic heterocycloalkyl, C₀₋₃alkylene-9- or 10-membered bicyclic heterocycloalkyl, C₀₋₃alkylene-5- or 6-membered monocyclic heteroaryl, or C₀₋₃alkylene-9- or 10-membered bicyclic heteroaryl;

and each R^{S11} is optionally substituted with one or more substituents independently selected from the group consisting of halo, oxo, cyano, C₀₋₃alkylene-OR^{p2}, C₀₋₃alkylene-S(═O)ₘR^{p2}, C₀₋₃alkylene-NR^{p2}R⁴, C₀₋₃alkylene-C(═O)NR^{p2}, C₀₋₃alkylene-C₀₋₃alkylene-C(═O)OR^{p2}, C₁₋₆alkyl, C₂₋₆alkenyl, C₂₋₆alkynyl, and C₁₋₆haloalkyl;

R³ is C₁₋₃alkyl, C₁₋₃haloalkyl, C₂₋₃alkenyl, C₂₋₃alkynyl, C₃₋₆cycloalkyl, —CN, —OR^{r}, —C(═O)R^{r}, —S(═O)ₘR^{r}, NR^{r}R^{t}, or —C(═O)OR^{r}, wherein C₁₋₃alkyl, C₂₋₃alkenyl and C₂₋₃alkynyl are optionally substituted with C₃₋₈cycloalkyl;

R⁴ is C₁₋₃alkyl, C₁₋₃haloalkyl, C₃₋₈cycloalkyl, phenyl, or 5- or 6-membered monocyclic heteroaryl, wherein cycloalkyl, phenyl and heteroaryl are optionally substituted with one or more substituents independently selected from the group consisting of halo, cyano, C₁₋₃alkyl, C₁₋₃haloalkyl, C₂₋₃alkenyl, C₂₋₃alkynyl, —OR^{w5}, and —NR^{w5}R^{x5};

each of R^{r} and R^{t}, independently, is H, C₁₋₃alkyl, C₁₋₃haloalkyl, C₂₋₃alkenyl, C₂₋₃alkynyl, C₃₋₆cycloalkyl, phenyl, 4- to 6-membered monocyclic heterocycloalkyl, or 5- or 6-membered monocyclic heteroaryl;

each of R^{c}, R^{c2}, R^{d}, R^{d′}, R^{d2}, R^{e}, R^{e2}, R^{f}, R^{f2}, Rⁿ, Rⁿ², R^{o}, R^{o2}, R^{p}, R^{p2}, and R^{q2}, independently, is H, C₁₋₃alkyl, C₁₋₃haloalkyl, C₃₋₈cycloalkyl, phenyl, 4- to 6-membered monocyclic heterocycloalkyl, or 5- or 6-membered monocyclic heteroaryl;

each R^{w}, R^{w5}, R^{x}, and R^{x5}, independently, is H, C₁₋₃alkyl, or C₁₋₃haloalkyl;

p is 0, 1, 2, 3, 4, or 5; and

m is 0, 1, or 2.

In another aspect, the present invention provides the compounds of Formula (III):

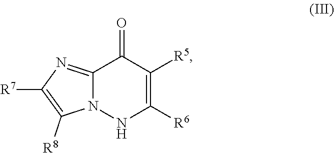

or a pharmaceutically acceptable salt, pharmaceutically acceptable solvate or pharmaceutically acceptable hydrate thereof. In this formula:

R⁵ is selected from the group consisting of —C(═O)NR⁹R¹⁰, phenyl optionally substituted with 1, 2, or 3 R¹¹, and a 5 to 10-membered heteroaryl optionally substituted with 1, 2, or 3 R¹²;

R⁶ is selected from the group consisting of —H, halo, —CN, C₁₋₃haloalkyl, and —C₁₋₃alkyl;

R⁷ is selected from the group consisting of —CN, —C₁₋₃alkyl, and C₁₋₃haloalkyl;

R⁸ is selected from the group consisting of phenyl optionally substituted with 1, 2, or 3 R¹³, and a 5- or 6-membered monocyclic heteroaryl optionally substituted with 1, 2, or 3 R¹⁴;

R⁹ and R¹⁰ are independently selected from the group consisting of —H, phenyl optionally substituted with 1, 2, or 3 R¹⁵, and a 5 to 10-membered heteroaryl optionally substituted with 1, 2, or 3 R¹⁶;

each R¹¹, R¹³ and R¹⁵ is independently selected from the group consisting of —C₁₋₃alkyl, —C₁₋₃haloalkyl, halo, —CN, —OH, —OC₁₋₃alkyl, and —OC₁₋₃haloalkyl; and

each R¹², R¹⁴ and R¹⁶ is independently selected from the group consisting of —C₁₋₃alkyl, —C₁₋₃haloalkyl, halo, —CN, —OH, —OC₁₋₃alkyl, and —OC₁₋₃haloalkyl.

In one embodiment of compounds of Formula (III), R⁵ is selected from the group consisting of —C(═O)NR⁹R¹⁰, phenyl optionally substituted with 1, 2, or 3 R¹¹, a 5- or 6-membered monocyclic heteroaryl optionally substituted with 1, 2, or 3 R¹², and a 9- or 10-membered bicyclic heteroaryl optionally substituted with 1, 2, or 3 R¹²; and one of R⁹ and R¹⁰ is —H and the other of R⁹ and R¹⁰ is selected from the group consisting of phenyl optionally substituted with 1, 2, or 3 R¹⁵, a 5- or 6-membered monocyclic heteroaryl optionally substituted with 1, 2, or 3 R¹⁶, and a 9- or 10-membered bicyclic heteroaryl optionally substituted with 1, 2, or 3 R¹⁶.

In one embodiment of compounds of Formula (III), R⁵ is selected from the group consisting of —C(═O)NR⁹R¹⁰, phenyl optionally substituted with 1, 2, or 3 R¹¹, a 5- or 6-membered monocyclic heteroaryl optionally substituted with 1, 2, or 3 R¹², and a 9- or 10-membered bicyclic heteroaryl optionally substituted with 1, 2, or 3 R¹²; one of R⁹ and R¹⁰ is —H and the other of R⁹ and R¹⁰ is selected from the group consisting of phenyl optionally substituted with 1, 2, or 3 R¹⁵, a 5- or 6-membered monocyclic heteroaryl optionally substituted with 1, 2, or 3 R¹⁶, and a 9- or 10-membered bicyclic heteroaryl optionally substituted with 1, 2, or 3 R¹⁶; R⁶ is C₁₋₃haloalkyl or —C₁₋₆alkyl, preferably —CH₃; R⁷ is —CN or —CF₃; and R⁸ is phenyl optionally substituted with 1, 2, or 3 substituents independently selected from the group consisting of —C₁₋₃alkyl, —C₁₋₃haloalkyl, halo, —CN, —OC₁₋₃alkyl, and —OC₁₋₃haloalkyl, preferably where R⁸ is unsubstituted phenyl.

In another aspect, conjugates are provided comprising compounds of the invention linked to a suitable ligand. In one embodiment, compounds of Formula I can be modified by replacing or modifying the R³ or R⁴ substituent to provide a suitable substituent comprising a reactive group capable of binding to a suitable linker. In some embodiments, the reactive group comprises a suitable hydroxy or amine group (e.g. an R³ or R⁴ substituent or modification thereof comprising a terminal —OH, —NH₂, C(═O)NH₂, and the like) that is capable of reacting with a suitable linker. In one embodiment, compounds of Formula I can be modified by replacing or modifying the R³ or R⁴ substituent, to provide a suitable substituent bound to a linker moiety, wherein said linker moiety comprises a reactive group capable of binding to a suitable ligand. In one embodiment, compounds of Formula I can be modified by replacing or modifying the R³ or R⁴ substituent to provide a suitable substituent bound to a linker moiety, wherein said linker moiety is bound to a suitable ligand. In one embodiment, the ligand binds to an E3 ubiquitin ligase. In some embodiments, the E3 ubiquitin ligase is MDM2, cIAP1, CRBN, or VHL. In one embodiment, a modified compound of the invention is a compound of Formula (Va), or Formula (Vb):

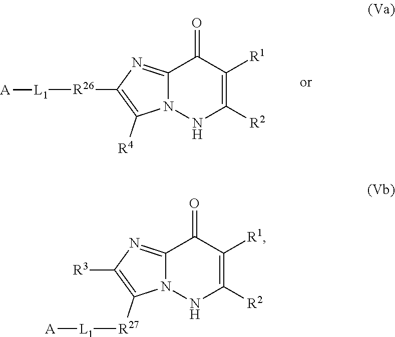

or a pharmaceutically acceptable salt, pharmaceutically acceptable solvate or pharmaceutically acceptable hydrate thereof. In these formulae, A is an E3 ubiquitin ligase ligand; L₁ is a suitable linker, R²⁶ is a suitable R³ or modification or replacement of R³ (as defined in Formula I), and R²⁷ is a suitable R⁴ or modification or replacement of R⁴ (as defined in Formula I); and R¹, R², R³, and R⁴ are as defined for compounds of Formula (I).

In reference to the present invention comprising a compound as disclosed herein, the compound includes a compound of Formula I (including Ia), Formula II (i.e. including IIa, IIb, IIc, IId, IIe, and IIf), or Formula III and all embodiments thereof, including any pharmaceutically acceptable salt, pharmaceutically acceptable solvate or pharmaceutically acceptable hydrate thereof.

The present invention provides a pharmaceutical composition comprising a compound as disclosed herein, including any pharmaceutically acceptable salt, pharmaceutically acceptable solvate or pharmaceutically acceptable hydrate thereof together with a pharmaceutically acceptable diluent or carrier. In some embodiments, the composition comprises a Janus Kinase (Jak) inhibitor, including a Jak1, Jak2, Jak3 or Tyk2 inhibitor, or compound that inhibits any combination thereof.

The present provides a kit comprising a compound as disclosed herein, or a pharmaceutically acceptable salt, pharmaceutically acceptable solvate or pharmaceutically acceptable hydrate thereof, including any container, pack, or dispenser together with instructions for administration. In some embodiments, the kit comprises a Janus Kinase (Jak) inhibitor, including a Jak1, Jak2, Jak3 or Tyk2 inhibitor, or compound that inhibits any combination thereof.

The present invention provides a compound as disclosed herein, including any pharmaceutically acceptable salt, pharmaceutically acceptable solvate or pharmaceutically acceptable hydrate thereof, for use in the treatment of a cGAS/STING pathway-mediated condition. In some embodiments, the use comprises administering a therapeutically effective amount of a Janus Kinase (Jak) inhibitor, including a Jak1, Jak2, Jak3 or Tyk2 inhibitor, or compound that inhibits any combination thereof.

The present invention provides a method of inhibiting the cGAS/STING pathway in a cell, comprising contacting the cell with one or more compounds or compositions of the present invention.

The present invention provides a method of inhibiting cytokine production in a cell, comprising contacting the cell with one or more compounds or compositions of the present invention, including any pharmaceutically acceptable salt, pharmaceutically acceptable solvate or pharmaceutically acceptable hydrate thereof.

The present invention provides a method of treating a cGAS/STING pathway-mediated condition, comprising administering to a subject in need thereof a therapeutically effective amount of one or more compounds or compositions of the present invention, including any pharmaceutically acceptable salt, pharmaceutically acceptable solvate or pharmaceutically acceptable hydrate thereof.

The present invention provides a method of treating a cGAS/STING pathway-mediated condition, comprising administering to a subject in need thereof a therapeutically effective amount of one or more compounds or compositions of the present invention, including any pharmaceutically acceptable salt, pharmaceutically acceptable solvate or pharmaceutically acceptable hydrate thereof, in combination with a Janus Kinase (Jak) inhibitor, including a Jak1, Jak2, Jak3 or Tyk2 inhibitor, or compound that inhibits any combination thereof.

For example, the cGAS/STING pathway-mediated condition is an autoimmune, inflammatory, or neurodegenerative condition. For example, wherein the disease is selected from the group consisting of systemic inflammatory response syndrome (SIRS), sepsis, septic shock, atherosclerosis, celiac disease, dermatomyositis, scleroderma, interstitial cystitis, transplant rejection (e.g. graft-versus-host disease). Aicardi-Goutieres Syndrome. Hutchison Guilford progeria syndrome, Singleton-Merten Syndrome, proteasome-associated autoinflammatory syndrome, SAVI (STING-associated vasculopathy with onset in infancy); CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenic purpura, thrombotic thrombocytopenic purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjogren's syndrome, X-linked reticulate pigmentary disorder, polymyositis, spondyloenchondrodysplasia, age-related macular degeneration, Alzheimer's disease and Parkinson's disease.

For example, wherein the disease is SIRS, sepsis, septic shock, atherosclerosis, celiac disease, interstitial cystitis, transplant rejection, Aicardi-Goutieres Syndrome, chilblain lupus erythematosus, systemic lupus erythematosus, idiopathic thrombocytopenic purpura, thrombotic thrombocytopenic purpura, autoimmune thrombocytopenia, spondyloenchondrodysplasia, psoriasis, Type 1 diabetes, Type 2 diabetes, or Sjogren's syndrome.

A method of treating an inflammatory disease in a subject, comprising administering to the subject in need thereof a therapeutically effective amount of one or more compounds or compositions of the present invention, including any pharmaceutically acceptable salt, pharmaceutically acceptable solvate or pharmaceutically acceptable hydrate thereof.

For example, wherein the disease is rheumatoid arthritis, juvenile rheumatoid arthritis, inflammatory bowel disease (ulcerative colitis, Crohn's disease), age-related macular degeneration, IgA nephropathy, glomerulonephritis, vasculitis, polymyositis, or Wegener's disease.

Another aspect of the invention provides a method of treating neurodegenerative diseases in a subject, comprising administering to the subject in need thereof a therapeutically effective amount of one or more compounds or compositions of the present invention, including any pharmaceutically acceptable salt, pharmaceutically acceptable solvate or pharmaceutically acceptable hydrate thereof.

For example, wherein the disease is Alzheimer's disease, Parkinson's disease, multiple sclerosis, IgM polyneuropathies, or myasthenia gravis.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

Other features and advantages of the invention will be apparent from the following detailed description and claims.

### DETAILED DESCRIPTION

STING (STimulator of INterferon Genes) is a central mediator for a cytosolic pathway that triggers type I interferon, in response to sensing cytosolic double-stranded (ds) DNA from infectious pathogens or aberrant host cells (Danger Associated Molecular Patterns, DAMPS) (Barber, Immunol. Rev 243: 99-108, 2011). Alternatively known as TMEM 173, MITA, ERIS, and MPYS, STING was discovered using cDNA expression cloning methods as a MyD88-independent host cell defense factor expressed in macrophages, dendritic cells (DCs) and fibroblasts was found to induce expression of IFN-3 and NF-κB dependent pro-inflammatory cytokines in response to sensing cytoplasmic DNA, in response to infection with herpes simplex virus (Ishikawa and Barber, Nature 455: 674-79, 2008).

While STING was discovered as being the critical sensor for inducing the production of IFN-β in response to infection with herpes simplex virus, the mechanism for this sensing function initially remained elusive. This conundrum was solved with the discovery of cyclic GMP-AMP synthase (cGAS), a host cell nucleotidyl transferase that directly binds dsDNA, and in response synthesizes a second messenger, c[G(2′,5′)pA(3′,5′)p] (cyclic GMP-AMP or 2′3′-cGAMP), which activates the STING pathway and induces IFN-β expression (Sun et al., Science 339: 786-91, 2013; Wu et al., Science 339: 826-30, 2013). This 2′3′-cGAMP product differed from bacterial-derived canonical cyclic dinucleotides, which were shown to respond differently to single nucleotide polymorphisms in the hSTING gene (Diner et al., Cell Reports 3:1355-1361, 2013; Gao et al., Cell 154:748-762, 2013; Conlon et, al., J Immunol 190:5216-5225, 2013). It was demonstrated that, while the bacterial-derived cyclic dinucleotides contained bis-3′-5′ linkages, cGAS produces a non-canonical, i.e., mixed linkage, CDN represented as c[G(2′,5′)pA(3′,5′)p] (Diner et al., Cell Reports 3:1355-1361, 2013; Gao et al., Cell 153:1094-1107, 2013; Ablasser et al., Nature 498: 380-84, 2013; Kranzusch et al., Cell Reports 3: 1362-68, 2013; Zhang et al., Mol. Cell. 51: 226-35, 2013). Cells without a functional cGAS are unable to express IFN-3 in response to stimulation with cytosolic DNA.

Given the role of cGAS in the STING pathway and the role of type I interferons in various diseases, treatment with a cGAS/STING pathway inhibitor may have therapeutic benefit in a number of inflammatory, autoimmune, and neurodegenerative diseases, including, but are not limited to, systemic inflammatory response syndrome (SIRS), sepsis, septic shock, atherosclerosis, celiac disease, dermatomyositis, scleroderma, interstitial cystitis, transplant rejection (e.g. graft-versus-host disease). Aicardi-Goutieres Syndrome, Hutchison Guilford progeria syndrome, Singleton-Merten Syndrome, proteasome-associated autoinflammatory syndrome, SAVI (STING-associated vasculopathy with onset in infancy), CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenic purpura, thrombotic thrombocytopenic purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjogren's syndrome, X-linked reticulate pigmentary disorder, polymyositis, spondyloenchondrodysplasia, age-related macular degeneration. Alzheimer's disease and Parkinson's disease. See, for example, Krienkamp et al., Cell Reports 22:2006-2015, 2018; Kerur et al., Nature Medicine 24:50-61, 2018; Yang et al., PNAS 114 (23): E4612-E4620, 2017; King et al., Nature Medicine 23:1481-1487, 2017; Bai et al., PNAS 114 (46):12196-12201, 2017; Ahn et al., Cell Reports 21:3873-3884, 2017; Li et al., J. Experimental Medicine, 215(5) 1287, 2018. In some embodiments, compounds of the invention are useful in treating Aicardi-Goutieres Syndrome, X-linked reticulate pigmentary disorder, dermatomyositis, systemic lupus erythematosus, rheumatoid arthritis, multiple sclerosis, or Type I or Type II diabetes.

The present invention provides novel pyrazolopyrimidinone or triazolopyrimidinone compounds, synthetic methods for making the compounds, pharmaceutical compositions containing them and various uses of the compounds.

### Imidazopyridazinone Compounds

The present invention provides the compounds of Formula (I):

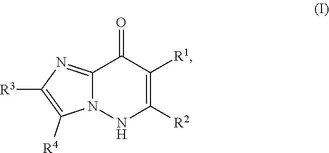

or a pharmaceutically acceptable salt, pharmaceutically acceptable solvate or pharmaceutically acceptable hydrate thereof. In this formula:

R¹ is Q¹-T¹-(X¹)ₙ;

Q¹ is a bond or C₁₋₃alkylene, wherein the C₁₋₃alkylene group is optionally substituted with one or more substituents independently selected from the group consisting of halo, C₁₋₆alkyl, C₂₋₆alkenyl, C₂₋₆alkynyl, cyano, C₁₋₆haloalkyl, —OR^{w2}, and —NR^{w2}R^{x2};

T¹ is C₃₋₈cycloalkyl, C₆₋₁₀aryl, 3- to 12-membered heterocycloalkyl, 5- to 10-membered heteroaryl, —C(═O)C₀₋₃alkylene-C₃₋₈cycloalkyl, —C(═O)—C₀₋₃alkylene-C₆₋₁₀aryl, —C(═O)—C₀₋₃alkylene-3 to 12-membered heterocycloalkyl, —C(═O)—C₀₋₃alkylene-5 to 10-membered heteroaryl, —NR^{a}R^{b}, —S(═O)₂R^{a}, —NR^{a}C(═O)R^{a}, —NR^{a}C(═O)NR^{a}R^{b}, —NR^{a}C(═O)OR^{a}, —NR^{a}S(═O)₂R^{a}, —C(═O)NR^{a}S(═O)₂R^{a}, —NR^{a}S(═O)₂NR^{a}R^{b}, —C(═O)NR^{a}R^{b}, or —S(═O)₂NR^{a}R^{b};

each X¹ is independently selected from the group consisting of halo, cyano, oxo, C₀₋₃alkylene-C(═O)R^{c}, C₀₋₃alkylene-OR^{c}, C₀₋₃alkylene-C(═O)OR, C₀₋₃alkylene-OC(═O)R^{c}, C₀₋₃alkylene-NR^{c}R^{d}, C₀₋₃alkylene-N⁺R^{c}R^{d}R^{d′}, C₀₋₃alkylene-S(═O)ₘR^{c}, C₀₋₃alkylene-NR^{c}C(═O)R^{c}, C₀₋₃alkylene-NR^{c}C(═O)NR^{c}R^{d}, C₀₋₃alkylene-OC(═O)NR^{c}R^{d}, C₀₋₃alkylene-NR^{c}C(═O)OR, C₀₋₃alkylene-NR^{c}S(═O)₂R^{c}, C₀₋₃alkylene-C(═O)NR^{c}S(═O)₂R^{c}, C₀₋₃alkylene-NR^{c}S(═O)₂NR^{c}R^{d}, C₀₋₃alkylene-C(═O)NR^{c}R^{d}, C₀₋₃alkylene-S(═O)₂NR^{c}R^{d}, C₀₋₃alkylene-C(═NR^{c})NR^{c}R^{d}, C₀₋₃alkylene-NR^{c}C(═NR^{c})NR^{c}R^{d}, and R^{S1}, in which R^{S1} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S1} is optionally substituted with one or more substituents independently selected from the group consisting of halo, cyano, nitro, oxo, C₁₋₆alkyl, C₂₋₆alkenyl, C₂₋₆alkynyl, C₁₋₆haloalkyl, C₀₋₃alkylene-NR^{e}R^{f} C₀₋₃alkylene-OR^{e}, C₀₋₃alkylene-NR^{e}C(═O)R^{e}, C₀₋₃alkylene-NR^{e}C(═O)OR^{e}, C₀₋₃alkylene-NR^{e}C(═O)NR^{e}R^{f}, C₀₋₃alkylene-OC(═O)R^{e}, C₀₋₃alkylene-C(═O)OR, C₀₋₃alkylene-C(═O)NR^{e}R^{f}, C₀₋₃alkylene-C(═O)R^{e}, C₀₋₃alkylene-S(═O)ₘR^{e}, C₀₋₃alkylene-S(═O)₂NR^{e}R^{f}, C₀₋₃alkylene-NR^{e}S(═O)₂R^{e}, C₀₋₃alkylene-C(═O)NR^{e}S(═O)₂R^{e}, C₀₋₃alkylene-NR^{e}S(═O)₂NR^{e}R^{f}, and R^{S2}, in which R^{S2} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl,

and each R^{S2} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w}, and —NR^{w}R^{x};

R² is Q²-T²-(X²)ₚ;

Q² is a bond or C₁₋₃alkylene, wherein the C₁₋₃alkylene group is optionally substituted with one or more substituents independently selected from the group consisting of halo, C₁₋₆alkyl, C₂₋₆alkenyl, C₂₋₆alkynyl, cyano, C₁₋₆haloalkyl, —OR^{w3}, and —NR^{w3}R^{x3};

T² is H, halo, cyano, C₆₋₁₀alkyl, C₂₋₆alkenyl, C₂₋₆alkynyl, C₃₋₈cycloalkyl, C₆₋₁₀aryl, 3- to 12-membered heterocycloalkyl, 5- to 10-membered heteroaryl, C(═O)—C₀₋₃alkylene-C₃₋₈cycloalkyl, C(═O)—C₀₋₃alkylene-C₆₋₁₀aryl, C(═O)—C₀₋₃alkylene-3- to 12-membered heterocycloalkyl, C(═O)—C₀₋₃alkylene-5- to 10-membered heteroaryl. —OR^{z}, —S(═O)ₘR^{k}, —P(═O)R^{kk}R^{mm}, —NR^{k}R^{m}, —C(═O)OR^{k}, or —C(═O)NR^{k}R^{m};

each X² is independently selected from the group consisting of halo, cyano, oxo, C₀₋₃alkylene-OR, C₀₋₃alkylene-S(═O)ₘRⁿ, C₀₋₃alkylene-NRⁿR^{o}, C₀₋₃alkylene-C(═O)NRⁿR^{o}, C₀₋₃alkylene-C(═O)ORⁿ, and R^{S3}, in which R^{S3} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and R^{S3} is optionally substituted with one or more substituents independently selected from the group consisting of halo, oxo, cyano, C₀₋₃alkylene-OR^{p}, C₀₋₃alkylene-S(═O)ₘR^{p}, C₀₋₃alkylene-NR^{p}R^{q}, C₀₋₃alkylene-C(═O)NR^{p}R^{q}, C₀₋₃alkylene-C₀₋₃alkylene-C(═O)OR^{p}, C₁₋₆alkyl, C₂₋₆alkenyl, C₂₋₆alkynyl, C₁₋₆haloalkyl, and R^{S4}, in which R^{S4} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S4} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w4}, and —NR^{w4}R^{x4};

R³ is C₁₋₆alkyl, C₁₋₃haloalkyl, C₂₋₃alkenyl, C₂₋₃alkynyl, C₃₋₈cycloalkyl, —CN, —OR^{r}, —C(═O)R^{r}, —S(═O)ₘR^{r}, NR^{r}R^{t}, or —C(═O)OR^{r}, wherein C₁₋₃alkyl, C₂₋₃alkenyl and C₂₋₃alkynyl are optionally substituted with one C₃₋₆cycloalkyl;

R⁴ is C₁₋₆alkyl, C₁₋₆haloalkyl, S(═O)ₘR^{u}, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl, wherein C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl are optionally substituted with one or more substituents independently selected from the group consisting of halo, oxo, C₁₋₆alkyl, C₂₋₆alkenyl, C₂₋₆alkynyl, cyano, C₁₋₆haloalkyl, OR^{w5}, and NR^{w5}R^{x5};

each of R^{a} and R^{b}, independently, is H or R^{S5}, in which R^{S5} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and R^{S5} is optionally substituted with one or more substituents independently selected from the group consisting of halo, cyano, oxo, C₁₋₆haloalkyl, C₀₋₃alkylene-OR^{c2}, C₀₋₃alkylene-C(═O)R^{c2}, C₀₋₃alkylene-C(═O)OR^{c2}, C₀₋₃alkylene-OC(═O)R^{c2}, C₀₋₃alkylene-C(═O)NR^{c2}R^{d2}, C₀₋₃alkylene-S(═O)ₘR^{c2}, C₀₋₃alkylene-S(═O)₂NR^{c2}R^{d2}, C₀₋₃alkylene-NR^{c2}R^{d2}, C₀₋₃alkylene-NR^{c2}C(═O)R^{c2}, C₀₋₃alkylene-NR^{c2}C(═O)OR^{c2}, C₀₋₃alkylene-NR^{c2}C(═O)NR^{c2}R^{d2}, C₀₋₃alkylene-NR²S(═O)₂R^{c2}, C₀₋₃alkylene-C(═O)NR^{c2}S(═O)₂R^{c2}, C₀₋₃alkylene-NR^{c2}S(═O)₂NR^{c2}R^{d2}, C₀₋₃alkylene-N(S(═O)₂R^{c2})₂, and R^{S6}, in which R^{S6} is C₁₋₆alkyl, C₂₋₆alkenyl, C₂₋₆alkynyl, C₀₋₃alkylene-C₃₋₄cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S6} is optionally substituted with one or more substituents independently selected from the group consisting of halo, cyano, oxo, C₁₋₆alkyl, C₂₋₆alkenyl, C₂₋₆alkynyl, C₁₋₆haloalkyl, C₀₋₃alkylene-NR^{e2}R^{f2}, C₀₋₃alkylene-OR^{e2}, C₀₋₃alkylene-NR^{e2}C(═O)R^{e2}, C₀₋₃alkylene-NR²C(═O)OR^{e2}, C₀₋₃alkylene-NR^{e2}C(═O)NR^{e2}R^{f2}, C₀₋₃alkylene-OC(═O)R², C₀₋₃alkylene-C(═O)OR², C₀₋₃alkylene-C(═O)NR^{e2}R^{f2}, C₀₋₃alkylene-C(═O)R^{e2}, C₀₋₃alkylene-S(═O)ₘR^{e2}, C₀₋₃alkylene-S(═O)₂NR^{e2}R^{f2}, C₀₋₃alkylene-NR^{e2}S(═O)R^{e2}, C₀₋₃alkylene-C(═O)NR^{e2}S(═O)₂R^{e2}, C₀₋₃alkylene-NR^{e2}S(═O)₂NR^{e2}R^{f2}, and R^{S7}, in which R^{S7} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S7} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w6}, and —NR^{w6}R^{x6};

each of R^{c}, R^{c2}, R^{d}, R^{d′}, and R^{d2}, independently, is H or R^{S5}, in which R^{S5} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S8} is optionally substituted with one or more substituents independently selected from the group consisting of halo, cyano, oxo, C₁₋₆alkyl, C₂₋₆alkenyl, C₂₋₆alkynyl, C₁₋₆haloalkyl, C₀₋₃alkylene-NR^{e3}R^{f3}, C₀₋₃alkylene-OR^{e3}, C₀₋₃alkylene-C(═O)OR^{e3}, C₀₋₃alkylene-C(═O)NR^{e3}R^{f3}, C₀₋₃alkylene-C(═O)R^{e3}, C₀₋₃alkylene-S(═O)ₘR^{e3}, C₀₋₃alkylene-S(═O)₂NR^{e3}R^{f3}, C₀₋₃alkylene-NR^{e3}C(O)R^{e3}, C₀₋₃alkylene-S(═O)₂NR^{e3}R^{f3}, C₀₋₃alkylene-NR^{f3}C(═O)R^{e3}, C₀₋₃alkylene-NR^{f3}S(═O)ₘR^{e3}, and R^{S9}, in which R^{S9} is C₀₋₃alkylene-C₃₋₈cycloalkyl, C₀₋₃alkylene-C₆₋₁₀aryl, C₀₋₃alkylene-3- to 12-membered heterocycloalkyl, C₀₋₃alkylene-5- to 10-membered heteroaryl;

and each R^{S9} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w7}, and —NR^{w7}R^{x7};

each of R^{e}, R^{e′}, R^{e3}, R^{f}, R^{f2}, and R^{f3}, independently, is H or R^{S10}, in which R^{S10} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S10} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w8}, and —NR^{w8}R^{x8};

each of R^{kk} and R^{mm}, is independently selected from the group consisting of R^{k}, —OR^{k}, and —NR^{k}R^{m};

each of R^{k} and R^{m}, independently, is H or R, in which R^{z} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{z} is optionally substituted with one or more substituents independently selected from the group consisting of halo, cyano, oxo, C₁₋₆alkyl, C₂₋₆alkenyl, C₂₋₆alkynyl, C₁₋₆haloalkyl, C₀₋₃alkylene-NRⁿ²R^{o2}, C₀₋₃alkylene-ORⁿ², C₀₋₃alkylene-C(═O)ORⁿ², C₀₋₃alkylene-C(═O)NRⁿ²R^{o2}, C₀₋₃alkylene-C(═O)Rⁿ², C₀₋₃alkylene-S(═O)ₘRⁿ², C₀₋₃alkylene-S(═O)₂NRⁿ²R^{o2}, and R^{S11}, in which R^{S11} is C₀₋₃alkylene-C₃₋₈cycloalkyl, C₀₋₃alkylene-C₆₋₁₀aryl, C₀₋₃alkylene-3- to 12-membered heterocycloalkyl, C₀₋₃alkylene-5- to 10-membered heteroaryl;

and each R^{S11} is optionally substituted with one or more substituents independently selected from the group consisting of halo, oxo, cyano, C₀₋₃alkylene-OR^{p}—, C₀₋₃alkylene-S(═O)ₘR^{p2}, C₀₋₃alkylene-NR^{p2}R^{q2}, C₀₋₃alkylene-C(═O)NR^{p2}R^{q2}, C₀₋₃alkylene-C₀₋₃alkylene-C(═O)OR^{p2}, C₁₋₆alkyl, C₂₋₆alkenyl, C₂₋₆alkynyl, C₁₋₆haloalkyl, and R^{S12}, in which R^{S12} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each R^{S12} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w9}, and —NR^{w9}R^{x9};

each of Rⁿ, Rⁿ², R^{o}, and R^{o2}, independently, is H or R^{S13}, in which R^{S13} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each R^{S13} is optionally substituted with one or more substituents independently selected from the group consisting of halo, oxo, cyano, C₀₋₃alkylene-OR^{p3}, C₀₋₃alkylene-S(═O)ₘR^{p3}, C₀₋₃alkylene-NR^{p3}R^{q3}, C₀₋₃alkylene-C(═O)NR^{p3}R^{q3}, C₀₋₃alkylene-C(═O)OR^{p3}, C₁₋₆alkyl, C₂₋₆alkenyl, C₂₋₆alkynyl, C₁₋₆haloalkyl, and R^{S14}, in which R^{S14} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each R^{S14} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w10}, and —NR^{w10}R^{x10};

each of R^{p}, R^{p2}, R^{p3}, R^{q}, R^{q2}, and R¹³, independently, is H or R^{S15}, in which R^{S15} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each R^{S15} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w11}, and —NR^{w11}R^{x11};

each of R^{r}, R^{t}, and R^{u}, independently, is H or R^{S16}, in which R^{S16} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl; and each R^{S16} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —C(═O)OR^{w12}, —OR^{w12}, and —NR^{w12}R^{x12};

each R^{w}, R^{w2}, R^{w3}, R^{w4}, R^{w5}, R^{w6}, R^{w7}, R^{w8}, R^{w9}, R^{w10}, R^{w11}, R^{w12}, R^{x}, R^{x2}, R^{x3}, R^{x4}, R^{x5}, R^{x6}, R^{x7}, R^{x8}, R^{x9}, R^{x10}, R^{x11}, and R^{x12}, independently, is H, C₁₋₆alkyl, C₂₋₆alkenyl, C₂₋₆alkynyl or C₁₋₆haloalkyl;

each of n and p independently is 0, 1, 2, 3, 4, or 5, wherein when T¹ is H, n is 0, and when T² is H, p is 0; and

m is 0, 1, or 2;

with the proviso that the compound is not

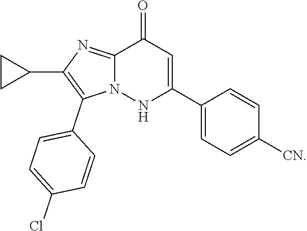 [SMILES: N#Cc1ccc(-c2cc(=O)c3nc(C4CC4)c(-c4ccc(Cl)cc4)n3[nH]2)cc1]

For example, Q¹ is a bond or —CH₂— and T¹ is C₃₋₈cycloalkyl, C₆₋₁₀aryl, 3- to 12-membered heterocycloalkyl, 5- to 10-membered heteroaryl, or —C(═O)NR^{a}R^{b}.

For example, Q¹ is a bond and T¹ is C₃₋₈cycloalkyl, C₆₋₁₀aryl, 3- to 12-membered heterocycloalkyl, or 5- to 10-membered heteroaryl.

For example, Q¹ is a bond and T¹ is C₆₋₁₀aryl, 3- to 12-membered heterocycloalkyl, or 5- to 10-membered heteroaryl.

For example Q¹ is a bond and T¹ is phenyl, 5- or 6-membered monocyclic heteroaryl, or 9- or 10-membered bicyclic heteroaryl, preferably wherein T¹ is 9- or 10-membered bicyclic heteroaryl.

For example, Q¹ is a bond or —CH₂—, T¹ is —C(═O)NR^{a}R^{b} and n is 0.

For example, one of R^{A} and R^{b} is H or methyl and the other of R and R^{b} is not H or methyl.

For example, R² is Q²-T²-(X²)ₚ, Q² is a bond, T² is H, halo, cyano, C₁₋₆alkyl, C₁₋₆haloalkyl, C₂₋₆alkenyl, C₂₋₆alkynyl, C₃₋₈cycloalkyl, C₆₋₁₀aryl, 3- to 12-membered heterocycloalkyl, or 5- to 10-membered heteroaryl, each X² independently is halo, cyano, oxo, C₀₋₃alkylene-ORⁿ, C₀₋₃alkylene-S(═O)ₘRⁿ, C₀₋₃alkylene-NRⁿR^{o}, C₀₋₃alkylene-C(═O)NRⁿR^{o}, C₀₋₃alkylene-C₀₋₃alkylene-C(═O)ORⁿ, and each Rⁿ and R^{o} is independently H, C₁₋₆alkyl, C₂₋₆alkenyl, C₂₋₆alkynyl or C₁₋₆haloalkyl.

For example, R² is Q²-T²-(X²)ₚ, Q² is a bond, T² is H, halo, cyano, C₁₋₆alkyl, C₁₋₆haloalkyl, C₂₋₆alkenyl, or C₂₋₆alkynyl, and each X² independently is halo or —OC₁₋₆alkyl.

For example, R² is H, cyano, methyl or methoxymethyl.

For example, R² is H, methyl or methoxymethyl.

For example, R³ is C₁₋₃alkyl, C₁₋₃haloalkyl, —CN, —S(═O)₂C₁₋₃alkyl or —C(═O)OC₁₋₃alkyl.

For example, R³ is —CN, C₁₋₃alkyl, C₁₋₃haloalkyl or —C(═O)OC₁₋₃alkyl.

For example, R³ is C₁₋₃alkyl, C₁₋₃haloalkyl or —C(═O)OC₁₋₃alkyl.

For example, R³ is —CF₃, methyl or —C(═O)OC₁₋₃alkyl.

For example, R³ is —CF₃ or —CN.

For example, R³ is —CF₃.

For example, R³ is —CN.

For example, R⁴ is C₁₋₃alkyl, C₁₋₃haloalkyl, —S(═O)₂C₁₋₃alkyl, C₃₋₈cycloalkyl, C₆₋₁₀aryl, 3- to 12-membered heterocycloalkyl, or 5- to 10-membered heteroaryl, wherein C₃₋₈cycloalkyl, C₆₋₁₀aryl, 3- to 12-membered heterocycloalkyl, or 5- to 10-membered heteroaryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}.

For example, R⁴ is C₃₋₈cycloalkyl, C₆₋₁₀aryl, 3 to 12-membered heterocycloalkyl, or 5- to 10-membered heteroaryl, wherein C₃₋₈cycloalkyl, C₆₋₁₀aryl, 3- to 12-membered heterocycloalkyl, or 5- to 10-membered heteroaryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w8}, and —NR^{w5}R^{x5}, wherein R^{w5} and R^{x5} are independently H, C₁₋₆alkyl or C₁₋₆haloalkyl.

For example, R⁴ is C₃₋₈cycloalkyl or C₆₋₁₀aryl, wherein C₃₋₈cycloalkyl and C₆₋₁₀aryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

For example, R⁴ is phenyl optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5} wherein R^{w5} and R^{x5} are independently H, C₁₋₆alkyl or C₁₋₆haloalkyl.

For example, R⁴ is C₃₋₈cycloalkyl.

For example, R⁴ is cyclopentyl.

For example, R⁴ is C₆₋₁₀aryl.

For example, R⁴ is phenyl.

For example, the compound can be of Formula (Ia):

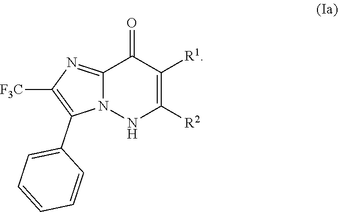

For example, Q¹ is a bond or —CH₂—.

For example, T¹ is —C(═O)—C₀₋₁alkylene-C₆₋₁₀aryl or —C(═O)—C₀₋₁alkylene-5 to 10-membered heteroaryl.

For example, Q¹ is a bond or —CH₂— and T¹ is C₃₋₈cycloalkyl, C₆₋₁₀aryl, 3- to 12-membered heterocycloalkyl, 5- to 10-membered heteroaryl, or —C(═O)NR^{a}R^{b}.

For example, Q¹ is a bond and T¹ is C₃₋₈cycloalkyl, C₆₋₁₀aryl, 3- to 12-membered heterocycloalkyl, or 5- to 10-membered heteroaryl.

For example, Q¹ is a bond and T¹ is C₆₋₁₀aryl, 3- to 12-membered heterocycloalkyl, or 5- to 10-membered heteroaryl.

For example, Q¹ is a bond and T¹ is phenyl, 5- or 6-membered monocyclic heteroaryl, or 9- or 10-membered bicyclic heteroaryl, preferably wherein T¹ is 9- or 10-membered bicyclic heteroaryl.

For example, T¹ is C(═O)NR^{a}R^{b} and n is 0.

For example, one of R^{a} and R^{b} is H or methyl and the other of R^{a} and R^{b} is not H or methyl.

For example, n is 0.

For example, T¹ is aryl or heteroaryl, preferably phenyl, 5- or 6-membered monocyclic heteroaryl, or 9- or 10-membered bicyclic heteroaryl.

For example, T¹ is 5- to 10-membered heteroaryl.

For example, T¹ is pyridinyl, pyrazinyl, pyridazinyl, pyrimidinyl, indolyl, indolinyl, isoindolyl, isoindolinyl, indazolyl, pyraolopyridinyl, prazolopyrimidinyl, oxazolopyrimidinyl, oxazolopyridinyl, imidazopyridinyl, benzimidazolyl, tetrahydrobenzimidazolyl, benzofuranyl, dihydrobenzofuranyl, isobenzofuranyl, dihydroisobenzofuranyl, triazolopyridinyl, benzothiazolyl, azabenzimidazolyl, azabenzoxazolyl, azabenzothiazolyl, quinolinyl, isoquinolinyl, quinazolinyl, oxazolyl, isoxazolyl, thiazolyl, isothiazolyl, benzoxazolyl, benzodioxolyl, chromanyl, tetrahydrooxazoloazepinyl, tetrahydrobenzoxazolyl, oxadiazolyl, thiadiazolyl, pyrazolyl, triazolyl, imidazolyl, furanyl, or thiophenyl.

For example, T¹ is pyridinyl, pyrazinyl, pyridazinyl, pyrimidinyl, indolyl, indazolyl, pyrazolopyridinyl, benzimidazolyl, benzothiazolyl, azabenzimidazolyl, azabenzoxazolyl, azabenzothiazolyl, imidazopyridinyl, quinolinyl, isoquinolinyl, quinazolinyl, oxazolyl, isoxazolyl, thiazolyl, isothiazolyl, benzoxazolyl, tetrahydrobenzoxazolyl, tetrahydrobenzimidazolyl, oxadiazolyl, thiadiazolyl, triazolyl, imidazolyl, furanyl, or thiophenyl.

For example, T¹ is pyridinyl, pyrazinyl, pyridazinyl, pyrimidinyl, indolyl, indazolyl, pyrazolopyridinyl, benzimidazolyl, benzothiazolyl, azabenzimidazolyl, azabenzoxazolyl, azabenzothiazolyl, imidazopyridinyl, quinolinyl, isoquinolinyl, quinazolinyl, oxazolyl, isoxazolyl, thiazolyl, isothiazolyl, benzoxazolyl, tetrahydrobenzoxazolyl, tetrahydrobenzimidazolyl, oxadiazolyl, thiadiazolyl, triazolyl, imidazolyl, furanyl, or thiophenyl, and X¹ is halo, C₀₋₃alkylene-OR, C₀₋₃alkylene-NR^{c}R^{d}, or R^{S1}, in which R^{S1} is C₁₋₆alkyl or C₀₋₃alkylene-C₃₋₈cycloalkyl.

For example, T¹ is C₀₋₁alkylene-C₆₋₁₀aryl.

For example, T¹ is phenyl, benzyl, naphthyl, or CH₂naphthyl.

For example, T¹ is 3- to 12-membered heterocycloalkyl, preferably 4- to 10-membered heterocycloalkyl.

For example, T¹ is piperazine, piperidine, quinuclidine, or morpholine.

For example, R² is Q²-T²-(X²)ₚ. Q² is a bond, T¹ is H, halo, cyano, C₁₋₆alkyl, C₁₋₆haloalkyl, C₂₋₆alkenyl, C₂₋₆alkynyl, C₃₋₈cycloalkyl, C₆₋₁₀aryl, 3- to 12-membered heterocycloalkyl, or 5 to 10-membered heteroaryl, each X² independently is halo, cyano, oxo, C₀₋₃alkylene-ORⁿ, C₀₋₃alkylene-S(═O)ₘRⁿ, C₀₋₃alkylene-NRⁿR^{o}, C₀₋₃alkylene-C(═O)NRⁿR^{o}, C₀₋₃alkylene-C₀₋₃alkylene-C(═O)ORⁿ, and each Rⁿ and R^{o} is independently H, C₁₋₆alkyl, C₂₋₆alkenyl, C₂₋₆alkynyl or C₁₋₆haloalkyl.

For example, R² is Q²-T²-(X²)ₚ, Q² is a bond, T² is H, halo, cyano, C₁₋₆alkyl, C₁₋₆haloalkyl, C₂₋₆alkenyl, or C₂₋₆alkynyl, and each X² independently is halo or OC₁₋₆alkyl.

For example, R² is H, cyano, methyl or methoxymethyl.

For example, R² is H, methyl or methoxymethyl.

Another subset of the compounds of Formula (I) includes those of Formula (IIa), or Formula (IIb):

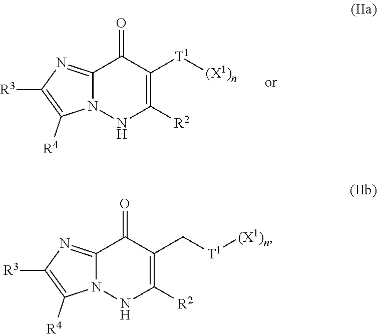

R², R³, R⁴, T¹, X¹ and n are as defined for Formula I.

In some embodiments of Formula IIa, or IIb, T¹ is C₃₋₈cycloalkyl, C₆₋₁₀aryl, 3- to 12-membered heterocycloalkyl, 5- to 10-membered heteroaryl, or C(═O)NR^{a}R^{b}.

In some embodiments of Formula IIa, T¹ is C₃₋₈cycloalkyl, C₆₋₁₀aryl, 3- to 12-membered heterocycloalkyl, or 5 to 10-membered heteroaryl.

In some embodiments of Formula IIa or IIb, T¹ is —C(═O)NR^{a}R^{b} and n is 0.

In some embodiments of Formula IIa or lib, R² is Q²-T²-(X²)ₚ, Q² is a bond, T² is H, halo, cyano, C₁₋₆alkyl, C₁₋₆haloalkyl, C₂₋₆alkenyl, C₂₋₆alkynyl, C₃₋₈cycloalkyl, C₆₋₁₀aryl, 3- to 12-membered heterocycloalkyl, or 5- to 10-membered heteroaryl, each X² independently is halo, cyano, oxo, C₀₋₃alkylene-ORⁿ, C₀₋₃alkylene-S(═O)ₘRⁿ, C₀₋₃alkylene-NRⁿR^{o}, C₀₋₃alkylene-C(═O)NRⁿR^{o}, C₀₋₁alkylene-C₀₋₃alkylene-C(═O)ORⁿ, and each Rⁿ and R^{o} is independently H, C₁₋₆alkyl, C₂₋₆alkenyl, C₂₋₆alkynyl or C₁₋₆haloalkyl.

In some embodiments of Formula IIa or IIb, R² is Q²-T²-(X²)ₚ, Q² is a bond, T² is H, halo, cyano, C₁₋₆alkyl, C₁₋₆haloalkyl, C₂₋₆alkenyl, or C₂₋₆alkynyl, and each X² independently is halo or —OC₁₋₆alkyl.

In some embodiments of Formula IIa or IIb, R² is H, cyano, methyl or methoxymethyl.

In some embodiments of Formula IIa or IIb, R² is H, methyl or methoxymethyl.

In some embodiments of Formula IIa or IIb, R³ is C₁₋₃alkyl, C₁₋₃haloalkyl, —CN, —S(═O)₂C₁₋₃alkyl or —C(═O)OC₁₋₃alkyl.

In some embodiments of Formula IIa or lib, R³ is C₁₋₃alkyl, C₁₋₆haloalkyl or —C(═O)OC₁₋₃alkyl.

In some embodiments of Formula IIa or IIb, R; is —CN, —CF₃, methyl or —C(═O)OC₁₋₃alkyl.

In some embodiments of Formula IIa or IIb, R³ is —CN or —CF₃.

In some embodiments of Formula IIa or IIb, R⁴ is C₁₋₃alkyl, C₁₋₃haloalkyl, —S(═O)₂C₁₋₃alkyl, C₃₋₈cycloalkyl, C₆₋₁₀aryl, 3- to 12-membered heterocycloalkyl, or 5- to 10-membered heteroaryl, wherein C₃₋₈cycloalkyl, C₆₋₁₀aryl, 3- to 12-membered heterocycloalkyl, or 5- to 10-membered heteroaryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}.

In some embodiments of Formula IIa or IIb, R⁴ is C₃₋₈cycloalkyl, C₆₋₁₀aryl, 3- to 12-membered heterocycloalkyl, or 5- to 10-membered heteroaryl, wherein C₃₋₈cycloalkyl, C₆₋₁₀-aryl, 3- to 12-membered heterocycloalkyl, or 5- to 10-membered heteroaryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

In some embodiments of Formula IIa or IIb, R⁴ is C₃₋₈cycloalkyl or C₆₋₁₀aryl, wherein C₃₋₈cycloalkyl and C₆₋₁₀aryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

In some embodiments of Formula IIa or IIb, R⁴ is C₃₋₈cycloalkyl.

In some embodiments of Formula IIa or IIb, R⁴ is cyclopentyl.

In some embodiments of Formula IIa or IIb, R⁴ is C₆₋₁₀aryl.

In some embodiments of Formula IIa or IIb, R⁴ is phenyl.

In some embodiments of Formula IIa or IIb, T¹ is C₃₋₈cycloalkyl, C₆₋₁₀aryl, 3- to 12-membered heterocycloalkyl, 5- to 10-membered heteroaryl, —C(═O)—C₀₋₃alkylene-C₃₋₈-cycloalkyl, —C(═O)—C₀₋₃alkylene-C₆₋₁₀aryl, —C(═O)—C₀₋₃alkylene-3- to 12-membered heterocycloalkyl, —C(═O)—C₀₋₃alkylene-5- to 10-membered heteroaryl, —NR^{a}R^{b}, —S(═O)₂R^{a}, —NR^{a}C(═O)R^{a}, —NR^{a}C(═O)NR^{a}R^{b}, —NR^{a}C(═O)OR^{a}, —NR^{a}S(═O)₂R^{a}, —C(═O)NR^{a}S(═O)₂R^{a}, —NR^{a}S(═O)₂NR^{a}R^{b}, —C(═O)NR^{a}R^{b}, or —S(═O)₂NR^{a}R^{b}; each X¹ independently is halo, cyano, oxo, C₀₋₃alkylene-C(═O)R^{c}, C₀₋₃alkylene-OR^{c}, C₀₋₃alkylene-NR^{c}R^{d}, C₀₋₃alkylene-OC(═O)NR^{c}R^{d}, C₀₋₃alkylene-C(═NR^{c})NR^{c}R^{d}, C₀₋₃alkylene-NR^{c}C(═NR^{c})NR^{c}R^{d}, or R^{S1}, in which R^{S1} is C₁₋₆alkyl, C₂₋₆alkenyl, C₂₋₆alkynyl, C₁₋₆haloalkyl, C₀₋₃alkylene-C₃₋₈cycloalkyl, C₀₋₃alkylene-C₆₋₁₀aryl, C₀₋₃alkylene-3 to 12-membered heterocycloalkyl, or C₀₋₃alkylene-5 to 10-membered heteroaryl, and R^{S1} is optionally substituted with one or more substituents independently selected from the group consisting of halo, oxo, C₁₋₆alkyl, C₂₋₆alkenyl, C₂₋₆alkynyl, C₁₋₆haloalkyl, C₀₋₃alkylene-NR^{e}R^{f}, C₀₋₃alkylene-OR^{e}, C₀₋₃alkylene-NR^{e}C(═O)R^{e}, C₀₋₃alkylene-NR^{e}C(═O)OR^{e}, C₀₋₃alkylene-NR^{e}C(═O)NR^{e}R^{f}, C₀₋₃alkylene-OC(═O)R^{e}, C₀₋₃alkylene-C(═O)OR^{e}, C₀₋₃alkylene-C(═O)R^{e}, C₀₋₃alkylene-S(═O)ₘR^{e}, C₀₋₃alkylene-S(═O)₂NR^{e}R^{f}, C₀₋₃alkylene-NR^{e}S(═O)₂R^{e}, C₀₋₃alkylene-NR^{e}S(═O)₂NR^{e}R^{f}, and R^{S2}, in which R^{S2} is C₀₋₃alkylene-C₃₋₈-cycloalkyl, C₀₋₃alkylene-C₆₋₁₀aryl, C₀₋₃alkylene-3- to 12-membered heterocycloalkyl, C₀₋₃alkylene-5- to 10-membered heteroaryl, and R^{S2} is optionally substituted with one or more substituents independently selected from the group consisting of halo, C₁₋₆alkyl, C₂₋₆alkenyl, C₂₋₆alkynyl, cyano, C₁₋₆haloalkyl, —OR^{w}, and —NR^{w}R^{x}.

Yet another subset of the compounds of Formula I includes those of Formula (IIc), or Formula (IId):

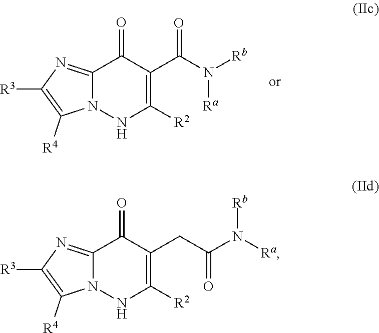

wherein R², R³, R⁴, R^{a} and R^{b} are as defined for Formula I.

In some embodiments of Formula IIc or IId, R² is Q²-T²-(X²)ₚ, Q² is a bond, T² is H, halo, cyano, C₁₋₆alkyl, C₁₋₆haloalkyl, C₂₋₄alkenyl, C₂₋₆alkynyl, C₃₋₈cycloalkyl, C₆₋₁₀aryl, 3 to 12-membered heterocycloalkyl, or 5 to 10-membered heteroaryl, each X² independently is halo, cyano, oxo, C₀₋₃alkylene-ORⁿ, C₀₋₃alkylene-S(═O)ₘRⁿ, C₀₋₃alkylene-NRⁿR^{o}, C₀₋₃alkylene-C(═O)NRⁿR^{o}, C₀₋₃alkylene-C₀₋₃alkylene-C(═O)ORⁿ, and each Rⁿ and R^{o} is independently H, C₁₋₆alkyl, C₂₋₆alkenyl, C₂₋₆alkynyl or C₁₋₆haloalkyl.

In some embodiments of Formula IIc or IId, R² is Q²-T²-(X²)ₚ, Q² is a bond, T² is H, halo, cyano, C₁₋₆alkyl, C₁₋₆haloalkyl, C₂₋₆alkenyl, or C₂₋₆alkynyl, and each X² independently is halo or OC₁₋₆alkyl.

In some embodiments of Formula IIc or IId, R² is H, cyano, methyl or methoxymethyl.

In some embodiments of Formula IIc or IId, R² is H, methyl or methoxymethyl.

In some embodiments of Formula IIc or IId, R³ is C₁₋₃alkyl, C₁₋₃haloalkyl, —CN, —S(═O)₂C₁₋₃alkyl or —C(═O)OC₁₋₃alkyl.

In some embodiments of Formula IIc or IId, R³ is —CN, C₁₋₃alkyl, C₁₋₃haloalkyl or —C(═O)OC₁₋₃alkyl.

In some embodiments of Formula IIc or IId, R³ is C₁₋₃alkyl, C₁₋₃haloalkyl or —C(═O)OC₁₋₆alkyl.

In some embodiments of Formula IIc or IId, R³ is —CN. —CF₃, methyl or —C(═O)OC₁₋₃alkyl.

In some embodiments of Formula IIc or IId, R³ is —CN or —CF₃.

In some embodiments of Formula IIc or IId, R⁴ is C₁₋₃alkyl, C₁₋₃haloalkyl, —S(═O)₂C₁₋₃alkyl, C₃₋₄cycloalkyl, C₆₋₁₀aryl, 3- to 12-membered heterocycloalkyl, or 5- to 10-membered heteroaryl, wherein C₃₋₈cycloalkyl, C₆₋₁₀aryl, 3- to 12-membered heterocycloalkyl, or 5- to 10-membered heteroaryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}.

In some embodiments of Formula IIc or IId, R⁴ is C₃₋₈cycloalkyl, C₆₋₁₀aryl, 3- to 12-membered heterocycloalkyl, or 5 to 10-membered heteroaryl, wherein C₃₋₈cycloalkyl, C₆₋₁₀aryl, 3 to 12-membered heterocycloalkyl, or 5- to 10-membered heteroaryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

In some embodiments of Formula IIc or IId, R⁴ is C₃₋₈cycloalkyl or C₆₋₁₀aryl, wherein C₃₋₈cycloalkyl and C₆₋₁₀aryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

In some embodiments of Formula IIc or IId, R⁴ is C₃₋₈cycloalkyl.

In some embodiments of Formula IIc or IId, R⁴ is cyclopentyl.

In some embodiments of Formula IIc or IId, R⁴ is C₆₋₁₀aryl.

In some embodiments of Formula IIc or IId, R⁴ is phenyl.

Still another subset of the compounds of Formula (I) includes those of Formula (IIe); or Formula (IIf);

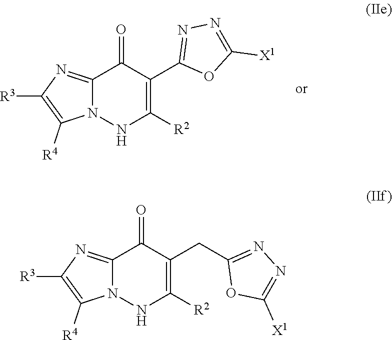

wherein each X¹ independently is halo, cyano, C₀₋₃alkylene-C(═O)R, C₀₋₃alkylene-OR^{c}, C₀₋₃alkylene-C(═O)OR^{c}, C₀₋₃alkylene-OC(═O)R^{c}, C₀₋₃alkylene-NR^{c}R^{d}, C₀₋₃alkylene-S(═O)ₘR^{c}, C₀₋₃alkylene-NR^{c}C(═O)R^{c}, C₀₋₃alkylene-NR^{c}C(═O)NR^{c}R^{d}, C₀₋₃alkylene-OC(═O)NR^{c}R^{d}, C₀₋₃alkylene-NR^{c}C(═O)OR^{c}, C₀₋₃alkylene-NR^{c}S(═O)₂R^{c}, C₀₋₃alkylene-C(═O)NR^{c}S(═O)₂R^{c}, C₀₋₃alkylene-NR^{c}S(═O)₂NR^{c}R^{d}, C₀₋₃alkylene-C(═O)NR^{c}R^{d}, C₀₋₃alkylene-S(═O)₂NR^{c}R^{d}, C₀₋₃alkylene-C(═NR)NR^{c}R^{d}, C₀₋₃alkylene-NR^{c}C(═NR^{c})NR^{c}R^{d}, or R^{S1}, in which R^{S1} is C₁₋₆alkyl, C₂₋₆alkenyl, C₂₋₆alkynyl, C₀₋₃alkylene-C₃₋₈cycloalkyl, C₀₋₃alkylene-C₆₋₁₀ aryl, C₀₋₃alkylene-3- to 12-membered heterocycloalkyl, or C₀₋₃alkylene-5- to 10-membered heteroaryl, R², R³, R⁴, R^{c}, R^{d} and R^{S1} are as defined for Formula I.

In some embodiments of Formula IIe, IIe′, IIf or IIf′, R² is Q²-T²-(X²)ₚ, Q² is a bond, T² is H, halo, cyano, C₁₋₆alkyl, C₁₋₆haloalkyl, C₂₋₆alkenyl, C₂₋₆alkynyl, C₃₋₈cycloalkyl, C₆₋₁₀-aryl, 3 to 12-membered heterocycloalkyl, or 5 to 10-membered heteroaryl, each X² independently is halo, cyano, oxo, C₀₋₃alkylene-ORⁿ, C₀₋₃alkylene-S(═O)ₘRⁿ, C₀₋₃alkylene-NRⁿR^{o}, C₀₋₃alkylene-C(═O)NRⁿR^{o}, C₀₋₃alkylene-C₀₋₃alkylene-C(═O)OR, and each Rⁿ and R^{o} is independently H, C₁₋₆alkyl, C₂₋₆alkenyl, C₂₋₆alkynyl or C₁₋₆haloalkyl.

In some embodiments of Formula IIe or IIf, R² is Q²-T²-(X²)ₚ, Q² is a bond, T² is H, halo, cyano, C₁₋₆alkyl, C₁₋₆haloalkyl, C₂₋₆alkenyl, or C₂₋₆alkynyl, and each X² independently is halo or —OC₁₋₆alkyl.

In some embodiments of Formula IIe or IIf, R² is H, cyano, methyl or methoxymethyl.

In some embodiments of Formula IIe or IIf, R² is H, methyl or methoxymethyl.

In some embodiments of Formula IIe or IIf, R³ is C₁₋₆alkyl, C₁₋₃haloalkyl, —CN, S(═O)₂C₁₋₃alkyl or —C(═O)OC₁₋₃alkyl

In some embodiments of Formula IIe or IIf, R³ is —CN, C₁₋₃alkyl, C₁₋₃haloalkyl or —C(═O)OC₁₋₃alkyl.

In some embodiments of Formula IIe or IIf, R³ is C₁₋₃alkyl, C₁₋₃haloalkyl or —C(═O)OC₁₋₃alkyl.

In some embodiments of Formula IIe or IIf, R³ is —CN, —CF₃, methyl or —C(═O)OC₁₋₃alkyl.

In some embodiments of Formula IIe or IIf, R³ is —CN or —CF₃.

In some embodiments of Formula IIe or IIf, R⁴ is C₁₋₃alkyl, C₁₋₃haloalkyl, S(═O)₂C₁₋₃alkyl, C₃₋₈cycloalkyl, C₆₋₁₀aryl, 3- to 12-membered heterocycloalkyl, or 5- to 10-membered heteroaryl, wherein C₃₋₈cycloalkyl, C₆₋₁₀aryl, 3- to 12-membered heterocycloalkyl, or 5- to 10-membered heteroaryl are optionally substituted with 1-3 substituents selected from the group consisting of halo, oxo, C₆₋₁₀alkyl, C₂₋₆alkenyl, C₂₋₆alkynyl, cyano, C₁₋₆haloalkyl, —OR^{w5}, and —NR^{w5}R^{x5}.

In some embodiments of Formula IIe or IIf, R⁴ is C₃₋₈cycloalkyl, C₆₋₁₀aryl, 3 to 12-membered heterocycloalkyl, or 5 to 10-membered heteroaryl, wherein C₃₋₈cycloalkyl, C₆₋₁₀-aryl, 3 to 12-membered heterocycloalkyl, or 5 to 10-membered heteroaryl are optionally substituted with 1-3 substituents selected from the group consisting of halo, oxo, C₁₋₆-alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

In some embodiments of Formula IIe or IIf, R⁴ is C₃₋₈cycloalkyl or C₆₋₁₀aryl, wherein C₃₋₈cycloalkyl and C₆₋₁₀aryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, OR^{w5}, and NR^{w5}R^{x5}, wherein R^{w5} and R^{x5} are independently H, C₆₋₁₀alkyl or C₁₋₆haloalkyl.

In some embodiments of Formula IIe or IIf, R⁴ is C₃₋₈cycloalkyl.

In some embodiments of Formula IIe or IIf, R⁴ is cyclopentyl.

In some embodiments of Formula IIe or IIf, R⁴ is C₆₋₁₀aryl.

In some embodiments of Formula IIe or IIf, R⁴ is phenyl.

Any of the substituents described herein for any of R¹, R², R³, R⁴, R^{a}, R^{b}, R^{c}, R^{d}, R^{e}, R^{f}, R^{e}, R^{h}, Rⁱ, R^{j}, R^{k}, R^{m}, Rⁿ, R^{p}, R^{r}, R^{t}, R^{u}, R^{w}, R^{w2}, R^{x}, R^{z}, R^{x2}, R^{S1}, R^{S2}, R^{S3}, R^{S4}, Q¹, Q², T¹, T², X¹, and X² can be combined with any of the substituents described herein for one or more of the remainder of R¹, R², R³, R⁴, R^{a}, R^{b}, R^{c}, R^{d}, R^{e}, R^{f}, R^{e}, R^{h}, Rⁱ, R^{j}, R^{k}, R^{m}, Rⁿ, R^{p}, R^{r}, R^{t}, R^{u}, R^{w}, R^{w2}, R^{z}, R^{x2}, R^{S1}, R^{S2}, R^{S3}, R^{S4}, Q¹, Q², T¹, T², X¹, and X².

In one embodiment, R¹, Q¹, T¹, X¹, R^{a}, R^{b}, R^{c}, R^{d}, R^{e}, R^{f}, R^{g}, R^{h}, R^{S1}, R^{S2}, R^{w}, R^{w2}, R^{x}, R^{z}, and R^{x2} are each as defined, where applicable, in any of Formula I (including Ia). Formula II (i.e. including IIa, IIb, IIc, IId, IIe, and IIf).

For example, T¹ is C(═O)NR^{a}R^{b} and n is 0.

For example, one of R^{a} and R^{b} independently is 5- to 10-membered heteroaryl and the other is hydrogen.

For example, one of R^{a} and R^{b} independently is pyridinyl, pyrazinyl, pyridazinyl, pyrimidinyl, indolyl, indazolyl, benzimidazolyl, imidazopyridinyl, quinolinyl, isoquinolinyl, quinazolinyl, oxazolyl, isoxazolyl, thiazolyl, isothiazolyl, benzoxazolyl, oxadiazolyl, pyrazolyl, benzodioxolyl, dihydrobenzofuranyl, triazolyl, imidazolyl, furanyl, or thiophenyl, each of which is optionally substituted with one or more groups independently selected from cyano, C₁₋₆haloalkyl, C₀₋₃alkylene-S(═O)ₘR^{c2}, C₀₋₃alkylene-OR^{c2}, C₀₋₃alkylene-NR^{c2}R^{d2}, and R^{S1}, in which R^{S1} is C₁₋₆alkyl, C₀₋₃alkylene-C₃₋₈cycloalkyl, or C₀₋₃alkylene-C₆₋₁₀aryl.

For example, one of R^{a} and R^{b} independently is pyridinyl, pyrazinyl, pyridazinyl, pyrimidinyl, indolyl, indazolyl, benzimidazolyl, imidazopyridinyl, quinolinyl, isoquinolinyl, quinazolinyl, oxazolyl, isoxazolyl, thiazolyl, isothiazolyl, benzoxazolyl, oxadiazolyl, pyrazolyl, benzodioxolyl, dihydrobenzofuranyl, triazolyl, imidazolyl, furanyl, or thiophenyl, each of which is optionally substituted with one or more groups independently selected from cyano, —CF₃, —S(═O)₂CH₃, —OCH₃, —NH₂, and R^{S6}, in which R^{S6} is CH₃, i-propyl, cyclopropyl, cyclopentyl, cyclohexyl, or phenyl.

For example, one of R^{a} and R^{b} is C₀₋₁alkylene-C₆₋₁₀aryl.

For example, one of R^{a} and R^{b} independently is phenyl, benzyl, naphthyl, or CH₂naphthyl, each of which is optionally substituted with one or more groups independently selected from halo, cyano, CF₃, C₀₋₃alkylene-S(═O)ₘR^{c2}, C₀₋₃alkylene-NR^{c2}S(═O)₂R², C₀₋₃alkylene-N(S(═O)₂R^{c2})₂, C₀₋₃alkylene-NR^{c2}C(═O)R^{c2}, C₀₋₃alkylene-NR²C(═O)OR^{c2}, C₀₋₃alkylene-OR^{c2}, C₀₋₃alkylene-NR^{c2}R^{d2}, and R^{S6}, in which R^{S6} is C₁₋₆alkyl, C₀₋₃alkylene-OR², or R^{S7}, in which R^{S7} is C₀₋₃alkylene-C₃₋₈cycloalkyl, C₀₋₃alkylene-C₆₋₁₀aryl, or C₀₋₃alkylene-3- to 12-membered heterocycloalkyl.

For example, one of R^{a} and R^{b} independently is phenyl, —CH₂phenyl, naphthyl, or —CH₂naphthyl, each of which is optionally substituted with one or more groups independently selected from F, Cl, cyano, —CF₃, —S(═O)₂CH₃, —S(═O)₂i-propyl, —NHS(═O)₂CH₃, —NHS(═O)₂phenyl, —N(S(═O)₂CH₃)₂, —NHC(═O)CH₃, —NHC(═O)OCH₃, —OCH₃, —OCF₃, —Oi-propyl, —Ocyclopentyl, —OCH₂phenyl, —NH₂, —N(CH₃)₂, and R^{S6}, in which R^{S6} is —CH₃, —CH₂OCH₃, i-propyl, or R^{S7}, in which R^{S7} is cyclopropyl, cyclopentyl, cyclohexyl, phenyl, pyrrolidinyl, or piperidinyl.

For example, one of R^{a} and R^{b} independently is optionally substituted 5- to 9-membered heterocycloalkyl.

For example, one of R^{a} and R^{b} independently is, tetrahydrobenzimidazole, morpholine, tetrahydrofuran, tetrahydropyran, pyrrolidine, piperidine, or piperazine, each of which is optionally substituted with C₀₋₃alkylene-C(═O)R^{c2}, C₀₋₃alkylene-C(═O)OR^{c2}, or R^{S6}, in which R^{S6} is C₁₋₆alkyl.

For example, one of R^{a} and R^{b} independently is, tetrahydrobenzimidazole, morpholine, tetrahydrofuran, tetrahydropyran, pyrrolidine, piperidine, or piperazine, each of which is optionally substituted with —CH₃, —C(═O)CH₃, or —C(═O)Ot-butyl.

For example, one of R^{a} and R^{b} independently is C₅₋₆cycloalkyl and the other is hydrogen.

For example, each of R^{a} and R^{b} independently is cyclohexane or cyclopropane, each of which is optionally substituted with C₀₋₃alkylene-OR^{c2} or C₀₋₃alkylene-NR^{c2}R^{d2}.

For example, each of R^{a} and R^{b} independently is cyclohexane or cyclopropane, each of which is optionally substituted with —OH, —OCH₃, or —NH₂.

For example, X¹ is optionally substituted C₀₋₁alkylene-C₆₋₁₀aryl.

For example, X¹ is phenyl, benzyl, naphthyl, or CH₂naphthyl.

For example, X¹ is optionally substituted 5 to 10-membered heteroaryl.

For example, X¹ is benzoxazolyl, benzimidazolyl, pyridinyl, pyrazinyl, pyridazinyl, pyrimidinyl, indolyl, indazolyl, imidazopyridinyl, quinolinyl, isoquinolinyl, quinazolinyl, oxazolyl, isoxazolyl, thiazolyl, isothiazolyl, benzoxazolyl, oxadiazolyl, triazolyl, imidazolyl, furanyl, or thiophenyl, each of which can be optionally substituted with one or more substituents selected from oxo.

For example, X¹ is optionally substituted 5- to 9-membered heterocycloalkyl.

For example, X¹ is tetrahydrobenzoxazole, tetrahydrobenzimidazole, morpholine, tetrahydrofuran, tetrahydropyran, piperidine, pyrrolidine, or piperazine, each of which can be optionally substituted with one or more substituents independently selected from C₁₋₆alkyl, C₁₋₆haloalkyl, C₀₋₃alkylene-OR^{e}, C₀₋₃alkylene-C(═O)OR^{e}, or R^{S2}, in which R^{S2} is C₀₋₃alkylene-C₆₋₁₀aryl.

For example, X¹ is optionally substituted C₃₋₆cycloalkyl.

For example, X¹ is —OR^{c} or —C(═O)C₁₋₆alkyl.

For example, X¹ is C₁₋₃alkyl.

For example, X¹ is —OCF₃, —OC₁₋₃alkyl, —NH₂, —CN, —OH or halo.

For example, X¹ is C₀₋₁alkylene-C(═NR^{c})NR^{c}R^{d}. For example, X¹ is —C(═NH)NH₂.

For example, X¹ is C₀₋₁alkylene-NR^{c}C(═NR^{c})NR^{c}R^{d}. For example, X¹ is —NHC(═NH)NH₂.

For example, R² is Q²-T²-(X²)ₚ, Q² is a bond, T² is H, halo, cyano, C₁₋₆alkyl, C₁₋₆haloalkyl, C₂₋₆alkenyl, C₂₋₆alkynyl, C₃₋₈cycloalkyl, C₆₋₁₀aryl, 3- to 12-membered heterocycloalkyl, or 5- to 10-membered heteroaryl, each X² independently is halo, cyano, oxo, C₀₋₃alkylene-ORⁿ, C₀₋₃alkylene-S(═O)ₘRⁿ, C₀₋₃alkylene-NRⁿR^{o}, C₀₋₃alkylene-C(═O)NRⁿR^{o}, C₀₋₃alkylene-C₀₋₃alkylene-C(═O)ORⁿ, and each Rⁿ and R^{o} is independently H, C₁₋₆alkyl, C₂₋₆alkenyl, C₂₋₆alkynyl or C₁₋₆haloalkyl.

For example, R² is Q²-T²-(X²)ₚ, Q² is a bond, T² is H, halo, cyano, C₁₋₆alkyl, C₁₋₆haloalkyl, C₂₋₆alkenyl, or C₂₋₆alkynyl, and each X² independently is halo or —OC₁₋₆alkyl.

For example, R² is H, cyano, methyl or methoxymethyl.

For example, R² is H, methyl or methoxymethyl.

For example, R³ is C₁₋₃alkyl, C₁₋₃haloalkyl, —CN, —S(═O)₂C₁₋₃alkyl or C(═O)OC₁₋₃alkyl.

For example, R³ is —CN, C₁₋₃alkyl, C₁₋₃haloalkyl or —C(═O)OC₁₋₆alkyl.

For example, R³ is C₁₋₃alkyl, C₁₋₃haloalkyl or —C(═O)OC₁₋₃alkyl.

For example, R³ is CN, CF₃, methyl or —C(═O)OC₁₋₃alkyl.

For example, R³ is —CN or —CF₃.

For example, R⁴ is C₁₋₃alkyl, C₁₋₃haloalkyl, —S(═O)₂C₁₋₃alkyl, C₃₋₈cycloalkyl, C₆₋₁₀aryl, 3- to 12-membered heterocycloalkyl, or 5- to 10-membered heteroaryl, wherein C₃₋₈cycloalkyl, C₆₋₁₀aryl, 3- to 12-membered heterocycloalkyl, or 5- to 10-membered heteroaryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}.

For example, R⁴ is C₃₋₈cycloalkyl, C₆₋₁₀aryl, 3 to 12-membered heterocycloalkyl, or 5- to 10-membered heteroaryl, wherein C₃₋₈cycloalkyl, C₆₋₁₀aryl, 3- to 12-membered heterocycloalkyl, or 5- to 10-membered heteroaryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

For example, R⁴ is C₃₋₈cycloalkyl or C₆₋₁₀aryl, wherein C₃₋₈cycloalkyl and C₆₋₁₀aryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

For example, R⁴ is C₃₋₈cycloalkyl.

For example, R⁴ is cyclopentyl.

For example, R⁴ is C₆₋₁₀aryl.

For example, R⁴ is phenyl.

In some embodiments, for a compound of Formula I, or a pharmaceutically acceptable salt, pharmaceutically acceptable solvate or pharmaceutically acceptable hydrate thereof:

R¹ is Q¹-T¹-(X¹)ₙ;

Q¹ is a bond. —CH₂—, or —CH₂CH₂—;

T¹ is C₆₋₁₀aryl, 3- to 12-membered heterocycloalkyl, 5- to 10-membered heteroaryl, —C(═O)—C₀₋₃alkylene-3- to 12-membered heterocycloalkyl, —NR^{a}R^{b}, —NR^{a}C(═O)R^{a}, —C(═O)NR^{a}S(═O)₂R^{a}, or —C(═O)NR^{a}R^{b};

each X¹ independently is halo, cyano, oxo, C₀₋₃alkylene-OR^{c}, C₀₋₃alkylene-C(═O)OR, C₀₋₃alkylene-NR^{c}R^{d}, C₀₋₃alkylene-N⁺R^{c}R^{d}R^{d′}, C₀₋₃alkylene-S(═O)ₘR^{c}, C₀₋₃alkylene-NR^{c}C(═O)R^{c}, C₀₋₃alkylene-OC(═O)NR^{c}R^{d}, C₀₋₃alkylene-C(═O)NR^{c}R^{d}, C₀₋₃alkylene-C(═NR^{c})NR^{c}R^{d}, C₀₋₃alkylene-NR^{c}C(═NR)NR^{e}R^{d}, or R^{S1}, in which R^{S1} is C₁₋₆alkyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl,

and each R^{S1} is optionally substituted with one or more substituents independently selected from the group consisting of halo, cyano, nitro, oxo, C₁₋₆alkyl, C₁₋₆haloalkyl, C₀₋₃alkylene-NR^{e}R^{f}, C₀₋₃alkylene-OR^{e}, C₀₋₃alkylene-NR^{e}C(═O)R^{e}, C₀₋₃alkylene-C(═O)OR^{e}, C₀₋₃alkylene-C(═O)R^{e}, C₀₋₃alkylene-S(═O)ₘR^{e}, C₀₋₃alkylene-NR^{e}S(═O)₂R^{e}, and R^{S2}, in which R^{S2} is C₀₋₃alkylene-C₆₋₁₀aryl or C₀₋₃alkylene-3- to 12-membered heterocycloalkyl,

and each R^{S2} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w}, and —NR^{w}R^{x};

each of R^{a} and R^{b}, independently, is H or R^{S5}, in which R^{S5} is C₁₋₆alkyl, C₀₋₃alkylene-C₃₋₈cycloalkyl, C₀₋₃alkylene-C₆₋₁₀aryl, C₀₋₃alkylene-3 to 12-membered heterocycloalkyl, or C₀₋₃alkylene-5- to 10-membered heteroaryl,

and R^{S5} is optionally substituted with one or more substituents independently selected from the group consisting of halo, cyano, oxo, C₁₋₆haloalkyl, C₀₋₃alkylene-OR^{c2}, C₀₋₃alkylene-C(═O)R^{c2}, C₀₋₃alkylene-C(═O)OR^{c2}, C₀₋₃alkylene-S(═O)ₘR^{c2}, C₀₋₃alkylene-NR^{c2}R^{d2}, C₀₋₃alkylene-NR^{c2}C(═O)R^{c2}, C₀₋₃alkylene-NR^{c2}C(═O)OR^{c2}, C₀₋₃alkylene-NR^{c2}S(═O)₂R^{c2}, C₀₋₃alkylene-N(S(═O)₂R^{c2})₂, and R^{S6}, in which R^{S6} is C₁₋₆alkyl, C₀₋₃alkylene-C₃₋₈cycloalkyl, or C₀₋₃alkylene-3- to 12-membered heterocycloalkyl,

and each R^{S6} is optionally substituted with one or more substituents independently selected from the group consisting of C₀₋₃alkylene-C₃₋₈cycloalkyl C₀₋₃alkylene-NR^{e2}R^{f2}, C₀₋₃alkylene-OR²;

R² is Q²-T²-(X²)ₚ;

Q² is a bond, —CH₂—, or —CH₂CH₂—:

T² is H, halo, cyano, C₁₋₆alkyl, C₃₋₈cycloalkyl, 3- to 12-membered heterocycloalkyl, 5- to 10-membered heteroaryl, C(═O)-3 to 12-membered heterocycloalkyl, —OR, —S(═O)ₘR^{k}, —P(═O)R^{kk}R^{mm}, —NR^{k}R^{m}, —C(═O)OR^{k} or —C(═O)NR^{k}R^{m};

each X² independently is halo, cyano, oxo, C₀₋₃alkylene-ORⁿ, C₀₋₃alkylene-C(═O)NRⁿR^{o}, C₀₋₃alkylene-C(═O)ORⁿ or R^{S3}, in which R^{S3} is C₁₋₆alkyl optionally substituted with C₀₋₃alkylene-OR^{p};

each of R^{kk} and R^{mm}, is independently selected from the group consisting of R^{k}, —OR^{k}, and —NR^{k}R^{m};

each of R^{k}, and R^{m}, independently, is H or R^{z}, in which R^{z} is C₁₋₆alkyl, C₀₋₃alkylene-C₃₋₈cycloalkyl, C₀₋₃alkylene-3 to 12-membered heterocycloalkyl, or C₀₋₃alkylene-5 to 10-membered heteroaryl;

and each R^{z} is optionally substituted with one or more substituents independently selected from the group consisting of halo, C₀₋₃alkylene-NRⁿ²R^{o2}, C₀₋₃alkylene-ORⁿ², C₀₋₃alkylene-C(═O)ORⁿ², C₀₋₃alkylene-C(═O)NRⁿ²R^{o2}, and R^{S11}, in which R^{S11} is C₀₋₃alkylene-3 to 12-membered heterocycloalkyl,

and each R^{S11} is optionally substituted with one or more substituents independently selected from the group consisting of halo, oxo, cyano, C₀₋₃alkylene-OR^{p2}, C₀₋₃alkylene-S(═O)ₘR^{p2}, C₀₋₃alkylene-NR^{p2}R^{q2}, C₀₋₃alkylene-C(═O)NR^{p2}R^{q2}, C₀₋₃alkylene-C₀₋₃alkylene-C(═O)OR^{p2}, C₁₋₆alkyl, C₂₋₆alkenyl, C₂₋₆alkynyl, and C₁₋₆haloalkyl; R³ is C₁₋₃alkyl, C₁₋₃haloalkyl, C₂₋₃alkenyl, C₂₋₃alkynyl, C₃₋₆cycloalkyl, —CN, —OR^{r}, —C(═O)R^{r}, —S(═O)ₘR^{r}, —NR^{r}R^{t}, or —C(═O)OR^{t}, wherein C₁₋₃alkyl, C₂₋₃alkenyl and C₂₋₃alkynyl are optionally substituted with C₁₋₆cycloalkyl;

R⁴ is C₁₋₃alkyl, C₁₋₃haloalkyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl, wherein C₀₋₃alkylene-C₃₋₈cycloalkyl, C₀₋₃alkylene-C₆₋₁₀aryl, C₀₋₃alkylene-3 to 12-membered heterocycloalkyl, or C₀₋₃alkylene-5 to 10-membered heteroaryl are optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5};

each of R^{c}, R^{c2}, R^{d}, R^{d′}, and R^{d2}, independently, is H, C₁₋₆alkyl, C₁₋₆haloalkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each of R^{e}, R^{e2}, R^{f}, and R^{f2}, independently, is H, C₁₋₆alkyl, C₁₋₆haloalkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3 to 12-membered heterocycloalkyl, or C₀₋₃alkylene-5- to 10-membered heteroaryl;

each of Rⁿ, Rⁿ², R^{o}, and R^{o2}, independently, is H or R^{S13}, in which R^{S13} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl; and each R^{S13} is optionally substituted with one or more substituents independently selected from the group consisting of halo, oxo, cyano, C₀₋₃alkylene-OR^{p3}, C₀₋₃alkylene-S(═O)ₘR^{p3}, C₀₋₃alkylene-NR^{p3}R^{q3}, C₀₋₃alkylene-C(═O)NR^{p}R^{Q3}, C₀₋₃alkylene-C(═O)OR^{p3}, C₁₋₆alkyl, C₂₋₆alkenyl, C₂₋₆alkynyl, C₁₋₆haloalkyl, C₀₋₃alkylene-C₃₋₈cycloalkyl, C₀₋₃alkylene-C₆₋₁₀aryl, C₀₋₃alkylene-3- to 12-membered heterocycloalkyl, and C₀₋₃alkylene-5- to 10-membered heteroaryl;

each of R^{p}, R^{p2}, R^{p3}, R^{q2}, and R^{q3}, independently, is H, C₁₋₆alkyl, C₁₋₆haloalkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each of R^{r}, and R^{t}, independently, is H, C₁₋₆alkyl, C₁₋₆haloalkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each R^{w}, R^{w5}, R^{x}, and R^{x5}, independently, is H, C₁₋₆alkyl, C₂₋₆alkenyl, C₂₋₆alkynyl or C₁₋₆haloalkyl;

each of n and p independently is 0, 1, 2, 3, 4, or 5, wherein when T¹ is H, n is 0, and when T² is H, p is 0; and

m is 0, 1, or 2.

In one embodiment, for a compound of Formula I, or a pharmaceutically acceptable salt, pharmaceutically acceptable solvate or pharmaceutically acceptable hydrate thereof:

R¹ is —(CH₂)₀₋₁—C(═O)NR^{a}R^{b}; —CH₂CH₂—NR^{a}R^{b}; —CH₂CH₂—NR^{a}C(═O)R^{a}; —C(═O)NR^{a}S(═O)₂R^{a}; —(CH₂)₀₋₁—C₆₋₁₀aryl; —(CH₂)₀₋₁-5- to 6-membered monocyclic heteroaryl; —(CH₂)₀₋₁-9- to 10-membered bicyclic heteroaryl; a 4- to 6-membered monocyclic heterocycloalkyl; a 9- to 10-membered bicyclic heterocycloalkyl; —C(═O)-4- to 6-membered monocyclic heterocycloalkyl; —C(═O)-9- to 10-membered bicyclic heterocycloalkyl; wherein the aryl, heteroaryl, and heterocycloalkyl rings are optionally independently substituted with 1, 2, 3.4, or 5 X¹;

each X¹ independently is halo; cyano; oxo; C₁₋₆alkyl optionally substituted with one or more substituents independently selected from the group consisting of halo, C₀₋₃alkylene-NR^{e}R^{f}, C₀₋₃alkylene-OR^{e}, C₀₋₃alkylene-C(═O)OR^{e}, C₀₋₃alkylene-C₃₋₆cycloalkyl, C₀₋₃alkylene-C₆₋₁₀aryl, and C₀₋₃alkylene-4 to 6-membered heterocycloalkyl, wherein heterocycloalkyl is optionally independently substituted with one or more C₁₋₆alkyl; C₀₋₃alkylene-C₃₋₆cycloalkyl optionally substituted with one or more substituents independently selected from the group consisting of C₁₋₆alkyl, C₁₋₆haloalkyl, C₁₋₃alkylene-NR^{e}R^{f}, and C₀₋₃alkylene-OR^{e}; C₀₋₃alkylene-C₆₋₁₀aryl, wherein C₆₋₁₀aryl is optionally substituted with one or more substituents selected from the group consisting of halo, cyano, nitro, C₁₋₆alkyl, C₁₋₆haloalkyl, C₀₋₃alkylene-NR^{e}R^{f}, C₀₋₃alkylene-OR^{e}, C₀₋₃alkylene-C(═O)OR^{e}, C₀₋₃alkylene-C(═O)R^{e}, C₀₋₃alkylene-S(═O)ₘR^{e}, and C₀₋₃alkylene-NR^{e}S(═O)₂R^{e}; C₀₋₃alkylene-4- to 6-membered monocyclic heterocycloalkyl or 9- or 10-membered bicyclic heterocycloalkyl, wherein heterocycloalkyl is optionally substituted with one or more substituents independently selected from the group consisting of oxo, C₀₋₃alkylene-NR^{e}R^{f}, and C₀₋₃alkylene-OR^{e}; C₀₋₃alkylene-5- or 6-membered monocyclic heteroaryl or 9- or 10-membered bicyclic heteroaryl, wherein heteroaryl is independently optionally substituted with one or more C₀₋₃alkylene-OR^{e}; C₀₋₃alkylene-OR^{c}; C₀₋₃alkylene-C(═O)OR C₀₋₃alkylene-NR^{c}R^{d}; C₀₋₃alkylene-N⁺R^{c}R^{d}R^{d′}; C₀₋₃alkylene-S(═O)ₘR^{c}; C₀₋₃alkylene-NR^{c}C(═O)R^{c}; C₀₋₃alkylene-OC(═O)NR^{c}R^{d}; C₀₋₃alkylene-C(O)NR^{c}R^{d}; C₀₋₃alkylene-C(═NR^{c})NR^{c}R^{d}; or C₀₋₃alkylene-NR^{c}C(═NR^{c})NR^{c}R^{d};

each of R^{a} and R^{b}, independently, is H or R^{S5}, in which R^{S5} is C₁₋₆alkyl, C₀₋₃alkylene-C₃₋₈cycloalkyl, C₀₋₃alkylene-C₆₋₁₀aryl, C₀₋₃alkylene-4- to 6-membered monocyclic heterocycloalkyl, C₀₋₃alkylene-9- or 10-membered bicyclic heterocycloalkyl, C₀₋₃alkylene-5- or 6-membered monocyclic heteroaryl, or C₀₋₃alkylene-9- or 10-membered bicyclic heteroaryl;

and R^{S5} is optionally substituted with one or more substituents selected from the group consisting of halo, cyano, oxo, C₁₋₆haloalkyl, C₀₋₃alkylene-OR^{c2}, C₀₋₃alkylene-C(═O)R^{c2}, C₀₋₃alkylene-C(═O)OR^{c2}, C₁₋₃alkylene-S(═O)ₘR^{c2}, C₀₋₃alkylene-NR^{c2}R^{d2}, C₀₋₃alkylene-NR^{c2}C(═O)R^{c2}, C₀₋₃alkylene-NR^{c2}C(═O)OR^{c2}, C₀₋₃alkylene-NR^{c2}S(═O)₂R², C₀₋₃alkylene-N(S(═O)₂R^{c2})₂, and R^{S6}, in which R^{S6} is C₁₋₆alkyl, C₀₋₃alkylene-C₁₋₆cycloalkyl, or C₀₋₃alkylene-4- to 6-membered monocyclic heterocycloalkyl;

and each R^{S6} is optionally substituted with one or more C₀₋₃alkylene-C₃₋₆cycloalkyl, C₀₋₃alkylene-NR^{e2}R^{f2}, C₀₋₃alkylene-OR^{e2};

R² is Q²-T²-(X²)ₚ;

Q² is a bond, —CH₂—, or —CH₂CH₂—;

T² is H, halo, cyano, C₁₋₆alkyl, C₁₋₆cycloalkyl, 4- to 6-membered monocyclic heterocycloalkyl, 9- or 10-membered bicyclic heterocycloalkyl, 5- or 6-membered monocyclic heteroaryl, 9- or 10-membered bicyclic heteroaryl, C(═O)-4- to 6-membered monocyclic heterocycloalkyl, —OR^{z}, —S(═O)ₘR^{k}, —P(═O)R^{k}R^{m}, —NR^{k}R^{m}, —C(═O)OR^{k}, or —C(═O)NR^{k}R^{m};

each X² independently is halo, cyano, oxo, C₀₋₃alkylene-ORⁿ, C₀₋₃alkylene-C(═O)NRⁿR^{o}, C₀₋₃alkylene-C(═O)ORⁿ or C₁₋₆alkyl, wherein C₁₋₆alkyl is optionally substituted with one C₀₋₃alkylene-OR^{p};

each of R^{kk} and R^{mm} is independently selected from the group consisting of R^{k}, —OR^{k}, and —NR^{k}R^{m};

each of R^{k} and R^{m}, independently, is H or R^{z}, in which R^{z} is C₁₋₆alkyl, C₀₋₃alkylene-C₃₋₆cycloalkyl, C₀₋₃alkylene-4- to 6-membered monocyclic heterocycloalkyl, C₀₋₃alkylene-9- or 10-membered bicyclic heterocycloalkyl, C₀₋₃alkylene-5 or 6-membered monocyclic heteroaryl, or C₀₋₃alkylene-9- or 10-membered bicyclic heteroaryl;

and each R^{z} is optionally substituted with one or more substituents selected from the group consisting of halo, C₁₋₆alkyl, C₀₋₃alkylene-NRⁿ²R^{o2}, C₀₋₃alkylene-ORⁿ², C₀₋₃alkylene-C(═O)ORⁿ², C₀₋₃alkylene-C(═O)NRⁿ²R^{o2}, and R^{S11}, in which R^{S11} is C₀₋₃alkylene-4- to 6-membered monocyclic heterocycloalkyl, C₀₋₃alkylene-9- or 10-membered bicyclic heterocycloalkyl, C₀₋₃alkylene-5- or 6-membered monocyclic heteroaryl, or C₀₋₃alkylene-9- or 10-membered bicyclic heteroaryl;

and each R^{S11} is optionally substituted with one or more substituents independently selected from the group consisting of halo, oxo, cyano, C₀₋₃alkylene-OR^{p2}, C₀₋₃alkylene-S(═O)ₘR^{p2}, C₀₋₃alkylene-NR^{p2}R^{q2}, C₀₋₃alkylene-C(═O)NR^{p2}R^{q2}, C₀₋₃alkylene-C₀₋₃alkylene-C(═O)OR^{p2}, C₁₋₆alkyl, C₂₋₆alkenyl, C₂₋₆alkynyl, and C₁₋₆haloalkyl;

R³ is C₁₋₃alkyl, C₁₋₆haloalkyl, C₂₋₃alkenyl, C₂₋₃alkynyl, C₃₋₆cycloalkyl, —CN, —OR^{e}, —C(═O)R^{r}, —S(═O)ₘR^{r}, NR^{r}R^{t}, or —C(═O)OR^{t}, wherein C₁₋₃alkyl, C₂₋₃alkenyl and C₂₋₃alkynyl are optionally substituted with C₃₋₆cycloalkyl;

R⁴ is C₁₋₃alkyl, C₁₋₃haloalkyl, C₃₋₆cycloalkyl, phenyl, or 5- or 6-membered monocyclic heteroaryl, wherein cycloalkyl, phenyl and heteroaryl are optionally substituted with one or more substituents independently selected from the group consisting of halo, cyano, C₁₋₃alkyl, C₁₋₃haloalkyl, C₂₋₃alkenyl, C₂₋₃alkynyl, —OR^{w5}, and —NR^{w5}R^{x5};

each of R^{r} and R^{t}, independently, is H, C₁₋₃alkyl, C₁₋₃haloalkyl, C₂₋₃alkenyl, C₂₋₃alkynyl, C₃₋₆cycloalkyl, phenyl, 4- to 6-membered monocyclic heterocycloalkyl, or 5- or 6-membered monocyclic heteroaryl;

each of R, R^{c2}, R^{d}, R^{d′}, R^{d2}, R^{e}, R^{e2}, R^{f}, R^{f2}, Rⁿ, Rⁿ², R^{o}, R^{o2}, R^{p}, R^{p2}, and R^{q2}, independently, is H, C₁₋₃alkyl, C₁₋₃haloalkyl, C₃₋₈cycloalkyl, phenyl, 4- to 6-membered monocyclic heterocycloalkyl, or 5- or 6-membered monocyclic heteroaryl;

each R^{w}, R^{w5}, R^{x}, and R^{x5}, independently, is H, C₁₋₃alkyl, or C₁₋₃haloalkyl;

p is 0, 1, 2, 3, 4, or 5; and

m is 0, 1, or 2.

In another aspect, the present invention provides the compounds of Formula (III):

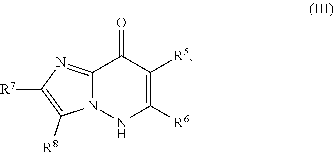

or a pharmaceutically acceptable salt, pharmaceutically acceptable solvate or pharmaceutically acceptable hydrate thereof. In this formula:

R⁵ is selected from the group consisting of —C(═O)NR⁹R¹⁰, phenyl optionally substituted with 1, 2, or 3 R¹¹, and a 5 to 10-membered heteroaryl optionally substituted with 1, 2, or 3 R¹²;

R⁶ is selected from the group consisting of —H, halo, —CN, C₁₋₆haloalkyl, and —C₁₋₃alkyl;

R⁷ is selected from the group consisting of —CN, —C₁₋₃alkyl, and C₁₋₃haloalkyl,

R⁸ is selected from the group consisting of phenyl optionally substituted with 1, 2, or 3 R¹³, and a 5- or 6-membered monocyclic heteroaryl optionally substituted with 1, 2, or 3 R¹⁴;

R⁹ and R¹⁰ are independently selected from the group consisting of —H, phenyl optionally substituted with 1, 2, or 3 R¹⁵, and a 5 to 10-membered heteroaryl optionally substituted with 1, 2, or 3 R¹⁶;

each R¹¹, R¹³ and R¹⁵ is independently selected from the group consisting of —C₁₋₃alkyl, —C₁₋₃haloalkyl, halo, —CN, —OH. —OC₁₋₆alkyl, and —OC₁₋₆haloalkyl; and each R¹², R¹⁴ and R¹⁶ is independently selected from the group consisting of —C₁₋₃alkyl, —C₁₋₃haloalkyl, halo, —CN, —OH, —OC₁₋₃alkyl, and —OC₁₋₃haloalkyl.

In some embodiments of compounds of Formula III, R⁵ is selected from the group consisting of —C(═O)NR⁹R¹⁰, phenyl optionally substituted with 1, 2, or 3 R¹¹, a 5- or 6-membered monocyclic heteroaryl optionally substituted with 1, 2, or 3 R¹¹, and a 9- or 10-membered bicyclic heteroaryl optionally substituted with 1, 2, or 3 R¹²; and one of R⁹ and R¹⁰ is —H and the other of R⁹ and R¹⁰ is selected from the group consisting of phenyl optionally substituted with 1, 2, or 3 R¹⁵, a 5- or 6-membered monocyclic heteroaryl optionally substituted with 1, 2, or 3 R¹⁶, and a 9- or 10-membered bicyclic heteroaryl optionally substituted with 1, 2, or 3 R¹⁶.

In some embodiments of compounds of Formula (III), R⁵ is selected from the group consisting of —C(═O)NR⁹R¹⁰, phenyl optionally substituted with 1, 2, or 3 R¹¹, a 5- or 6-membered monocyclic heteroaryl optionally substituted with 1, 2, or 3 R¹², and a 9- or 10-membered bicyclic heteroaryl optionally substituted with 1, 2, or 3 R¹²; one of R⁹ and R¹⁰ is —H and the other of R⁹ and R¹⁰ is selected from the group consisting of phenyl optionally substituted with 1, 2, or 3 R¹⁵, a 5- or 6-membered monocyclic heteroaryl optionally substituted with 1, 2, or 3 R¹⁶, and a 9- or 10-membered bicyclic heteroaryl optionally substituted with 1, 2, or 3 R¹⁶; R⁶ is C₁₋₃haloalkyl or —C₁₋₃alkyl, preferably —CH₃; R⁷ is —CN or —CF₃; and R⁸ is phenyl optionally substituted with 1, 2, or 3 substituents independently selected from the group consisting of —C₁₋₃alkyl, —C₁₋₃haloalkyl, halo, —CN, —OC₁₋₃alkyl, and —OC₁₋₃haloalkyl, preferably where R⁸ is unsubstituted phenyl.

As used herein, “alkyl”, “C₁, C₂, C₃, C₄, C₅ or C₆ alkyl” or “C₁₋₆alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intended to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, s-pentyl or n-hexyl. The term Cₘ₋ₙ means the alkyl group has “m” to “n” carbon atoms. The term “alkylene” refers to an alkyl group having a substituent, i.e. as used here in it is a bivalent alkyl moiety. For example C₀₋₃alkylene, as used herein as part of a substituent of another group includes a direct bond, a linear group —CH₂—, —CH₂CH₂—, or —CH₂CH₂CH₂—, or a branched group —CH(CH₃)—, —CH₂CH(CH₃)—, or —CH(CH₃)CH₂—, where C₂ or C₃ alkylene is preferably linear. For groups described as having more than one alkyl component, for example —N(C₁₋₃alkyl)₂, —C(═O)N(C₁₋₃alkyl)₂, —P(═O)(C₁₋₆alkyl)₂, or the like, the alkyl components may be the same or different. For example dialkylamino represents as —N(C₁₋₃alkyl)₂ includes N,N-dimethylamino, N,N-diethylamino, N-isopropyl-N-methyl-amino, and the like.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “cycloalkyl” refers to a saturated or unsaturated nonaromatic hydrocarbon mono- or multi-ring (e.g., fused, bridged, or spiro rings) system having 3 to 30 carbon atoms (e.g., C₃₋₁₀). For example, a C₃₋₈ cycloalkyl is intended to include a monocyclic, bicyclic or tricyclic ring having 3, 4, 5, 6, 7, or 8 carbon atoms. Examples of cycloalkyl rings include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, and adamantyl. Bridged rings are also included in the definition of cycloalkyl, including, for example, [3.3.0]bicyclooctane, [4.3.0]bicyclononane, [4.4.0]bicyclodecane and [2.2.2]bicyclooctane. A bridged ring occurs when one or more carbon atoms link two non-adjacent carbon atoms. In one embodiment, bridge rings are one or two carbon atoms. It is noted that a bridge always converts a monocyclic ring into a tricyclic ring. When a ring is bridged, the substituents recited for the ring may also be present on the bridge. Fused and spiro rings are also included. In the case of multicyclic rings, none of the rings is aromatic.

The term “heterocycloalkyl” refers to a saturated or unsaturated nonaromatic 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, or Se), unless specified otherwise. For example, a 3 to 14-membered heterocycloalkyl ring is intended to include a monocyclic, bicyclic, or tricyclic ring having 3, 4, 5, 6, 7, 8, 9, 10, 11, 12, 13, or 14 atoms selected from C, O, N, S, and Se. In another example, a 3 to 12-membered heterocycloalkyl ring is intended to include a monocyclic, bicyclic, or tricyclic ring having 3, 4, 5, 6, 7, 8, 9, 10, 11, 12 atoms selected from C, O, N, S, and Se. The nitrogen and sulfur heteroatoms may optionally be oxidized (i.e., N→O and S(═O)ₚ, where p=1 or 2). In the case of multicyclic rings, none of the rings is aromatic. Examples of heterocycloalkyl groups include, but are not limited to, piperidinyl, piperazinyl, pyrrolidinyl, dioxanyl, tetrahydrofuranyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, decahydroquinolinyl, 2H,6H-1,5,2-dithiazinyl, dihydrofuro[2,3-b]tetrahydrofuran, imidazolinyl, octahydroisoquinolinyl, oxazolinyl (dihydrooxazolyl), oxazolidinyl, piperidonyl, 4-piperidonyl, piperonyl, pyrazolidinyl, pyrazolinyl, pyrrolinyl, 2H-pyrrolyl, quinuclidinyl, 6H-1,2,5-thiadiazinyl, and the like.

Substituted alkyl is alkyl in which the designated substituents replace one or more hydrogen atoms on one or more carbons of the hydrocarbon backbone. Such substituents can include, for example, alkyl, alkenyl, alkynyl, halogen, hydroxyl, oxo, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

An “arylalkyl” or an “aralkyl” moiety is an alkyl substituted with an aryl (e.g., phenylmethyl (benzyl)). An “alkylaryl” moiety is an aryl substituted with an alkyl (e.g., methylphenyl).

As used herein, “alkylene linker” is intended to include C₁, C₂, C₃, C₄, C₅ or C₆ straight chain (linear) saturated divalent aliphatic hydrocarbon groups and C₂, C₃, C₄, C₅ or C₆ branched saturated aliphatic hydrocarbon groups. For example, C₁₋₃ alkylene linker as used in describing Q¹ and Q² of Formula I herein is a C₁₋₃ alkylene intended to include C₁, C₂, and C₃ alkyl linker groups. These linker groups bind to the core Formula I and to T¹ or T². Examples of alkylene linker include, moieties having from one to six carbon atoms, such as, but not limited to, methyl, ethyl, n-propyl, i-propyl, n-butyl, s-butyl, i-butyl, n-pentyl, s-pentyl, or n-hexyl.

“Alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂₋₆” or “C₂-C₆” includes alkenyl groups containing two to six carbon atoms. The term “C₃₋₆” or “C₃-C₆” includes alkenyl groups containing three to six carbon atoms.

Substituted alkenyl is alkenyl in which the designated substituents replace one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino); acylamino (including alkylcarbonylamino, arylcarbonylamino, carbamoyl and ureido); amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

“Alkynyl” includes unsaturated aliphatic groups analogous in length and possible substitution to the alkyl groups described above, but which contain at least one triple bond. For example, “alkynyl” includes straight chain alkynyl groups (e.g. ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g. C₂-C₆ for straight chain, C₃-C₆ for branched chain). The term “C₂₋₆” or “C₂-C₆” includes alkynyl groups containing two to six carbon atoms. The term “C₃₋₆” or “C₃-C₆” includes alkynyl groups containing three to six carbon atoms.

Substituted alkynyl is alkynyl in which the designated substituents replace one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino); acylamino (including alkylcarbonylamino, arylcarbonylamino, carbamoyl and ureido); amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

“Aryl” includes groups with aromaticity, including “conjugated,” or multicyclic systems with at least one aromatic ring and lacking a heteroatom in the ring structure. For example, a C₆₋₁₀aryl is intended to include a monocyclic, bicyclic or tricyclic ring having 6, 7, 8, 9, or 10 carbon atoms. Examples include phenyl, 1,2,3,4-tetrahydronaphthalenyl, naphthalene, etc.

“Heteroaryl” groups are aryl groups, as defined above, except having from one to four heteroatoms in the ring structure, and may also be referred to as “aryl heterocycles” or “heteroaromatics.” For example, a 5 to 10-membered heteroaryl ring is intended to include a stable 5-, 6-, 7-, 8-, or 9-membered monocyclic or 5-, 6-, 7-, 8-, 9-, or 10-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen, sulfur, selenium, and boron. The nitrogen atom may be substituted or unsubstituted (i.e., N or NR wherein R is H or other substituents, as defined). The nitrogen and sulfur heteroatoms may optionally be oxidized (i.e., N→O and S(═O)ₚ, where p=1 or 2). It is to be noted that total number of S and O atoms in the aromatic heterocycle is not more than 1. Preferred heteroaryl groups herein include 5- or 6-membered monocyclic heteroaryl or 9- or 10-membered bicyclic heteroaryl.

Examples of heteroaryl groups include pyrrolyl, furanyl, thiophenyl, thiazolyl, isothiazolyl, imidazolyl, triazolyl, tetrazolyl, pyrazolyl, oxazolyl, isoxazolyl, pyridinyl, pyrazinyl, pyridazinyl, pyrimidinyl, indolinyl, indolyl, 3H-indolyl, isoindolyl, isoquinolinyl, pyridazinyl, pyridooxazolyl, pyridoimidazolyl, pyridothiazole, pyridinyl, pyrimidinyl, oxadiazolyl (e.g. 1,2,3-oxadiazolyl, 1,2,4-oxadiazolyl, 1,2,5-oxadiazolyl, 1,3,4-oxadiazolyl), pyrazolopyridinyl, benzimidazolyl, tetrahydrobenzimidazolyl, benzothiazolyl, benzofuranyl, dihydrobenzofuranyl, pteridinyl, purinyl, pyrazinyl, benzothiofuranyl, benztriazolyl, benztetrazolyl, benzisoxazolyl, benzisothiazolyl, benzothiophenyl, benzoxazolyl, azabenzimidazolyl, azabenzoxazolyl, azabenzothiazolyl, thiadiazolyl (e.g. 1,2,4-thiadiazolyl, 1,2,5-thiadiazolyl, 1,3,4-thiadiazolyl, 1,2,3-thiadiazolyl), triazinyl, triazolyl (e.g. 1,2,3-triazolyl, 1,2,4-triazolyl, 1,2,5-triazolyl, 1,3,4-triazolyl), benzoxazolinyl, benzimidazolinyl, indolizinyl, quinazolinyl, 4H-quinolizinyl, quinoxalinyl, benzodioxolyl, benzoxazolyl, benzoxadiazolyl, tetrahydrobenzoxazolyl (e.g. 4,5,6,7-tetrahydrobenzo[d]oxazolyl); tetrahydrobenzimidazolyl (e.g. 4,5,6,7-tetrahydro-1H-benzo[d]imidazolyl), quinolinyl, isoquinolinyl, tetrahydroquinolinyl (e.g. 1,2,3,4-tetrahydroquinolinyl); tetrahydroisoquinolinyl (e.g. 1,2,3,4-tetrahydroisoquinolinyl), naphthridinyl, deazapurinyl, benzodihydropyranyl, imidazopyridinyl (e.g. imidazo[1,2-a]]pyridinyl), indazolyl, pyrazolopyrimidinyl (e.g. pyrazolo[1,5-a]pyrimidinyl), 5,6,7,8-tetrahydro-4H-cyclohepta[d]oxazolyl, oxazolopyrimidinyl (e.g. oxazolo[5,4-d]pyrimidinyl); oxazolopyridinyl (e.g. oxazolo[4,5-b]pyridinyl, oxazolo[5,4-b]pyridinyl, oxazolo[5,4-c]pyridinyl, oxazolo[4,5-c]pyridinyl), isobenzofuranyl, dihydroisobenzofuranyl, triazolopyridinyl, tetrahydrooxazoloazepinyl (e.g. 5,6,7,8-tetrahydro-4H-oxazolo[4,5-c]azepinyl), azocinyl, carbazolyl, 4aH-carbazolyl, carbolinyl, chromanyl, chromenyl, cinnolinyl, isoindolinyl, indolenyl, isatinonyl, isochromanyl, isoindazolyl, naphthyridinyl, thianthrenyl, thienothiazolyl, thienooxazolyl, thienoimidazolyl, xanthenyl, furazanyl, oxindolyl, phenanthridinyl, phenanthrolinyl, phenazinyl, phenothiazinyl, phenoxathinyl, phenoxazinyl, phthalazinyl, and the like.

Furthermore, the terms “aryl” and “heteroaryl” include multicyclic aryl and heteroaryl groups, e.g., tricyclic or bicyclic rings.

In the case of multicyclic aromatic rings, only one of the rings needs to be aromatic (e.g., 2,3-dihydroindole), although all of the rings may be aromatic (e.g., quinoline). The second ring can also be fused or bridged.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, oxo, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino); acylamino (including alkylcarbonylamino, arylcarbonylamino, carbamoyl and ureido); amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl).

The term “substituted,” as used herein, means that any one or more hydrogen atoms on the designated atom is replaced with a selection from the indicated groups, provided that the designated atom's normal valency is not exceeded, and that the substitution results in a stable compound. When a moiety is indicated as substituted with one or more substituents, this typically indicates substitution with 1, 2, 3, 4, 5, or more, including 1 to 5, 1 to 4, 1 to 3, 1 to 2 or 1 substituents independently selected from an indicated group. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., X¹) occurs more than one time in any constituent or formula for a compound, its definition at each occurrence is independent of its definition at every other occurrence. Thus, for example, if a group is shown to be substituted with 0-2 X¹ moieties, then the group may optionally be substituted with up to two X¹ moieties and X¹ at each occurrence is selected independently from the definition of X¹. Also, combinations of substituents and/or variables are permissible, but only if such combinations result in stable compounds.

The term “hydroxy” or “hydroxyl” includes groups with an —OH or —O⁻.

As used herein, “halo” or “halogen” refers to fluoro, chloro, bromo and iodo. The term “perhalogenated” generally refers to a moiety wherein all hydrogen atoms are replaced by halogen atoms. The term “haloalkyl” or “haloalkoxyl” refers to an alkyl or alkoxyl substituted with one or more halogen atoms. For example, C₁₋₆haloalkyl indicates a 1 to 6 carbon alkyl group (linear or branched) where one or more hydrogens is replaced with one or more halogen. For example, C₁₋₆haloalkyl includes —CF₃, —CHF₂, CH₂F, etc.

The term “carbonyl” includes compounds and moieties which contain a carbon connected with a double bond to an oxygen atom. Examples of moieties containing a carbonyl include, but are not limited to, aldehydes, ketones, carboxylic acids, amides, esters, anhydrides, etc.

The term “carboxyl” refers to —COOH or its C₁-C₆ alkyl ester.

“Acyl” includes moieties that contain the acyl radical (R—C(═O)—) or a carbonyl group. “Substituted acyl” includes acyl groups where one or more of the hydrogen atoms are replaced by, for example, alkyl groups, alkynyl groups, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Alkoxyalkyl,” “alkylaminoalkyl,” and “thioalkoxyalkyl” include alkyl groups, as described above, wherein oxygen, nitrogen, or sulfur atoms replace one or more hydrocarbon backbone carbon atoms.

The term “alkoxy” or “alkoxyl” includes substituted and unsubstituted alkyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, acylamino, diarylamino, and alkylarylamino), acylamino (including alkylcarbonylamino, arylcarbonylamino, carbamoyl and ureido); amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

The term “ester” includes compounds or moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. An example of keto-enol equilibria is between imidazo[1,2-b]pyridazin-8(5H)-one and the corresponding imidazo[1,2-b]pyridazin-8-ol, as shown below.

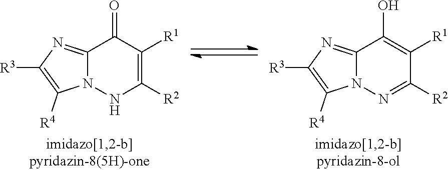

It is to be understood that the compounds of the present invention may be depicted as different tautomers. It should also be understood that when compounds have tautomeric forms, all tautomeric forms are intended to be included in the scope of the present invention, and the naming of the compounds does not exclude any tautomer form. It will be understood that certain tautomers may have a higher level of activity than others.

### Synthesis of Imidazopyridazinone Compounds

The present invention provides methods for the synthesis of the compounds of any Formula disclosed herein. The present invention also provides detailed methods for the synthesis of various disclosed compounds of the present invention according to the following schemes and further exemplified for specific compounds as shown in the Examples.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

The synthetic processes of the invention can tolerate a wide variety of functional groups, therefore various substituted starting materials can be used. The processes generally provide the desired final compound at or near the end of the overall process, although it may be desirable in certain instances to further convert the compound to a pharmaceutically acceptable salt, solvate, hydrate, ester, or prodrug thereof.

Compounds of the present invention can be prepared in a variety of ways using commercially available starting materials, compounds or methods known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition. John Wiley & Sons: New York, 2001; Greene, T. W., Wuts, P. G. M., Protective Groups in Organic Synthesis, 3^{rd} edition, John Wiley & Sons: New York, 1999; R Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995); incorporated by reference herein, are useful and recognized reference textbooks of organic synthesis known to those in the art. The following descriptions of synthetic methods are designed to illustrate, but not to limit, general procedures for the preparation of compounds of the present invention.

For example, compounds of the present invention can be prepared according to the processes illustrated below in Schemes 1-6.

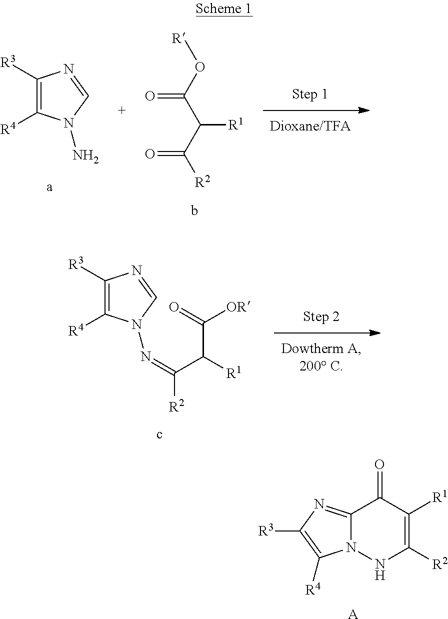

Scheme 1 shows the synthesis of a compound of Formula A, which can be a compound of Formula (I), where R¹, R², R³ and R⁴ are within the definitions of Formula I, and R′ is e.g. methyl, or ethyl. Substituted 1-amino-imidazoles (compound a), which are commercially available or can be made similarly to or according to methods as described in Yamada, M.; Fukui, T.; Nunami, K. Synthesis 1995, 1365, or by using other chemistry known to one skilled in the art, can b condensed with an appropriately substituted beta-keto ester (compound b) to give intermediate compound c. Compound c is heated with Dowtherm A® to provide compounds of Formula A, including compounds of Formula (I).

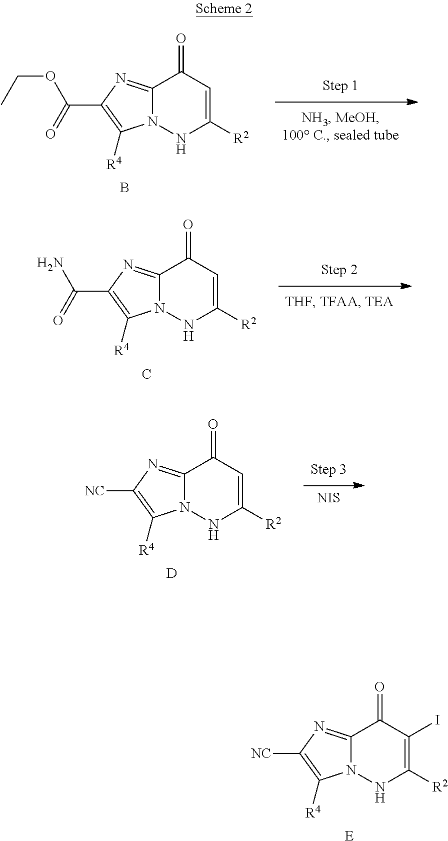

The compound of Formula B (per Scheme 1 wherein R¹ is H and R³ is C(═O)OEt), is heated in a sealed tube with NH₃ in MeOH to provide compounds of Formula C, which are reacted with trifluoroacetic anhydride and triethylamine in THF to provide compounds of Formula D. Compounds of Formula E are provided by reacting the compound of Formula D with N-iodosuccinimide in DMF at 0° C. The compounds of Formula B and C can be similarly reacted with N-iodosuccinimide to prepare the R¹=iodo analogs of those compounds. Analogs where R¹=bromo can be prepared by reacting with N-bromosuccinimide in place of N-iodosuccinimide.

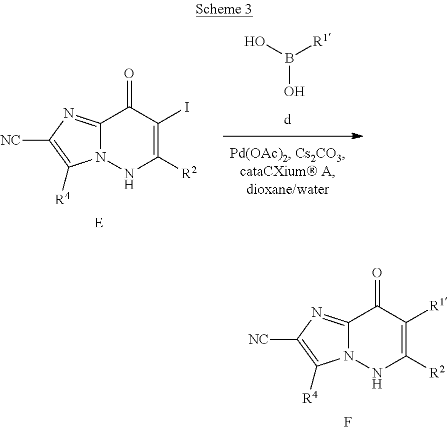

The Compound of Formula E, e.g. as prepared by Schemes 1 and 2, is reacted with a suitable boronic acid d (e.g. optionally substituted aryl or optionally substituted heteroaryl boronic acid) and cesium carbonate, cataCXium® A, and cesium carbonate in degassed dioxane-water heated under N₂ in a sealed tube. Compounds of Formula F are provided, wherein R^{1′} is e.g. an optionally substituted aryl or optionally substituted heteroaryl. The compound E or similar 7-iodo compounds where the 2 position is C₁₋₃alkyl or C₁₋₃haloalkyl (e.g. —CF₃, see Example 2 below) in place of —CN could also be similarly reacted with a suitable amine compound (NH₂R^{1″}) and including Mo(CO)₆ as a carbonyl source and DBU as a base for aminocarbonylation coupling to provide compounds where R^{1′}=—C(O)NH—R^{1″} (see Example 4 below).

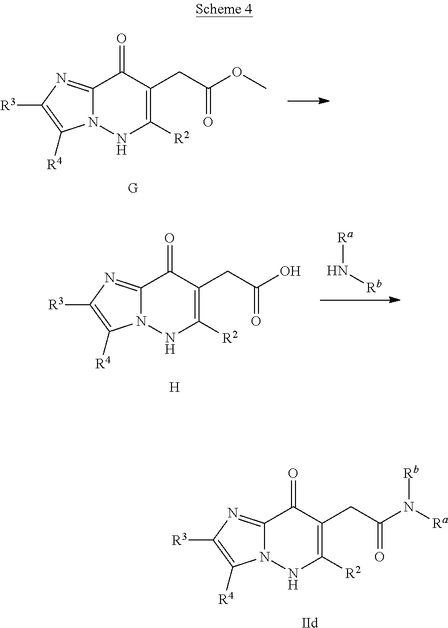

Scheme 4 shows the synthesis of imidazopyridazinones of Formulas (IId), where R², R³, R⁴, R^{a} and R^{b} are within the definitions of Formula (I). A compound of Formula G (prepared similarly to Scheme 1 wherein R¹ is CH₂C(═O)OMe) is saponified to produce the acid compound H. The acid can then be coupled to an NR^{a}R^{b} group to give compounds of Formula (IId). Compounds of Formulas (IIc) can be synthesized using a similar route, starting with a beta-keto ester such as dimethyl 2-acetylmalonate (e.g. Scheme 1 where R¹=C(═O)OMe).

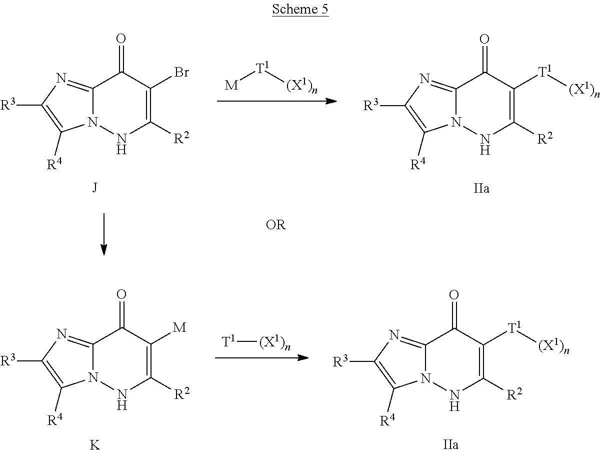

Scheme 5 shows the synthesis of some imidazopyridazinones of Formula (IIa), R², R³, R⁴, T¹ and X¹ are within the definitions of Formula (I). The compound of Formula J (prepared per Scheme 2) is reacted to provide the T¹-X¹ group, which can be introduced through a metal-catalyzed cross-coupling reaction with an M-T¹-X¹ group to give compounds of Formula (IIa). Alternatively, the brominated intermediate can be converted to a metalated nucleophile Formula K, which can then react with an electrophilic T¹-X¹ group to form compounds of Formula (IIa). “M” refers to metallic functional groups such as B(OH)₂, Sn(alkyl)₃, Si(alkyl)₃, MgBr, Li, CuLi, ZnCl, and the like.

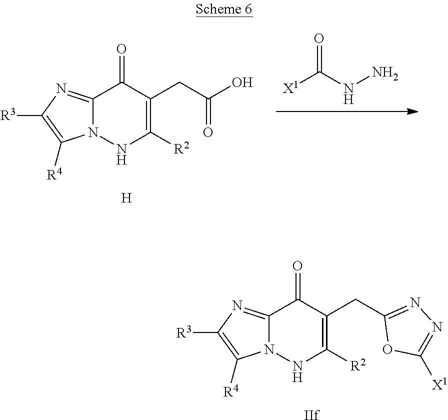

Scheme 6 shows the synthesis of compounds of Formula (IIf) from acid Compound H (see Scheme 4). Compound H can be treated with an appropriate hydrazide, e.g., acetylhydrazide, to give compounds of Formula (IIf). Compounds of Formula (IIe) can be synthesized using a similar route, starting from the acid resulting from the reaction of Scheme 4 where R¹=C(═O)OMe. Additional methods of making compounds of Formula (I) are found in the Examples below.

Compounds of the invention can also be used in the preparation of Proteolysis Targeting Chimeras (PROTACs), wherein the conjugates of the invention are conjugated to ligand that binds an E3 ubiquitin ligase via a suitable linker. For example, a compound of Formula I can be modified off of the R³ or R⁴ substituent position to provide a linker, which can be reacted to bind with a suitable ligand. Thus in one embodiment, a conjugate is provided comprising a compound of the invention that binds cGAS linked to a ligand of an E3 ubiquitin ligase, wherein the resulting conjugate binds to both the E3 ubiquitin ligase and cGAS. This results in the binding of ubiquitin to the cGAS protein by the E3 ubiquitin ligase. The resulting modified cGAS is then processed by the cell, resulting in degradation of the protein. Suitable ligands that bind E3 ubiquitin ligase, and suitable linkers, and methods of making such conjugates are well known to one skilled in the art. See, for example. Collins et al., Biochemical Journal 2017, 474:1127-1147; Bondeson, et al., Nature Chemical Biology 2015, 11:611-617; and Toure and Crews. Angew. Chem. Int. Ed. 2016, 55:2-10.

Thus, conjugates are provided comprising compounds of the invention linked to a suitable ligand. In one embodiment, compounds of Formula I can be modified by replacing or modifying the R³ or R⁴ substituent, to provide a suitable substituent comprising a reactive group capable of binding to a suitable linker. In some embodiments, the reactive group comprises a suitable hydroxy or amine group (e.g. an R³ or R⁴ substituent or modification thereof comprising a terminal —OH, —NH₂, C(═O)NH₂, and the like) that is capable of reacting with a suitable linker. In one embodiment, compounds of Formula I can be modified by replacing or modifying the R³ or R⁴ substituent, to provide a suitable substituent bound to a linker moiety, wherein said linker moiety comprises a reactive group capable of binding to a suitable ligand. In one embodiment, compounds of Formula I can be modified by replacing or modifying the R³ or R⁴ substituent, to provide a suitable substituent bound to a linker moiety, wherein said linker moiety is bound to a suitable ligand. In one embodiment, the ligand binds to an E3 ubiquitin ligase. In some embodiments, the E3 ubiquitin ligase is MDM2, cIAP1, CRBN, or VHL. In one embodiment, a modified compound of the invention is a compound of Formula (Va), or Formula (Vb);

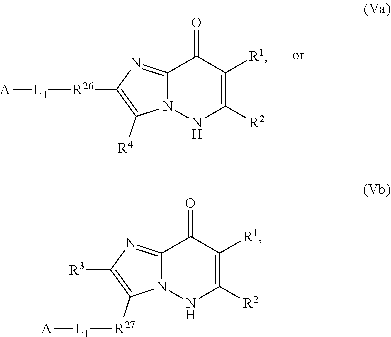

or a pharmaceutically acceptable salt, pharmaceutically acceptable solvate or pharmaceutically acceptable hydrate thereof. In these formulae, A is an E3 ubiquitin ligase ligand; Li is a suitable linker, R²⁶ is a suitable R³ or modification or replacement of R³ (as defined in Formula I), and R²⁷ is a suitable R⁴ or modification or replacement of R⁴ (as defined in Formula I); and R¹, R², R³, and R⁴ are as defined for compounds of Formula (I).

### Methods

The phrase “cGAS/STING pathway-mediated condition,” as used herein, comprises autoimmune, inflammatory, and neurodegenerative conditions. For example, the autoimmune disorder is selected from disease can be a type I interferonopathy (e.g., Aicardi-Goutieres Syndrome, Sjögren's syndrome, Singleton-Merten Syndrome, proteasome-associated autoinflammatory syndrome, SAVI (STING-associated vasculopathy with onset in infancy), CANDLE syndrome, chilblain lupus erythematosus, systemic lupus erythematosus, spondyloenchondrodysplasia), rheumatoid arthritis, juvenile rheumatoid arthritis, idiopathic thrombocytopenic purpura, autoimmune myocarditis, thrombotic thrombocytopenic purpura, autoimmune thrombocytopenia, psoriasis, Type 1 diabetes, or Type 2 diabetes. For example, the inflammatory disorder is selected from atherosclerosis, dermatomyositis, SIRS, sepsis, septic shock, celiac disease, interstitial cystitis, transplant rejection, inflammatory bowel disease (ulcerative colitis, Crohn's disease), age-related macular degeneration, IgA nephropathy, glomerulonephritis, vasculitis, polymyositis, or Wegener's disease.

Compound of the invention as described herein, can be useful in treating a variety of diseases, where the modulation of the cGAS/STING pathway can provide therapeutic benefit. In some aspects, a compound of the invention inhibits the cGAS/STING pathway, and can be useful in treating a disease selected from the group consisting of systemic inflammatory response syndrome (SIRS), sepsis, septic shock, atherosclerosis, celiac disease, dermatomyositis, scleroderma, interstitial cystitis, transplant rejection (e.g. graft-versus-host disease), Aicardi-Goutieres Syndrome, Hutchison Guilford progeria syndrome, Singleton-Merten Syndrome, proteasome-associated autoinflammatory syndrome, SAVI (STING-associated vasculopathy with onset in infancy), CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenic purpura, thrombotic thrombocytopenic purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjogren's syndrome, X-linked reticulate pigmentary disorder, polymyositis, spondyloenchondrodysplasia, age-related macular degeneration, Alzheimer's disease and Parkinson's disease. In some embodiments, compounds of the invention are useful in treating Aicardi-Goutieres Syndrome, X-linked reticulate pigmentary disorder, dermatomyositis, systemic lupus erythematosus, rheumatoid arthritis, multiple sclerosis, or Type I or Type II diabetes.

As used herein, “treating” or “treat” describes the management and care of a mammalian subject (e.g. human patient) for the purpose of combating a disease, condition, or disorder and includes the administration of a compound of the present invention, or a pharmaceutically acceptable salt, pharmaceutically acceptable solvate or pharmaceutically acceptable hydrate thereof, to alleviate the symptoms or complications of a disease, condition or disorder, or to eliminate the disease, condition or disorder. The term “treat” can also include treatment of a cell in vitro or an animal model.

A compound of the present invention, or a pharmaceutically acceptable salt, pharmaceutically acceptable solvate or pharmaceutically acceptable hydrate thereof, can also be used to prevent a disease, condition or disorder, or used to identify suitable candidates for such purposes. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the invention leads to the elimination of a sign or symptom, however, elimination is not required. Effective dosages are expected to decrease the severity of a sign or symptom. For instance, a sign or symptom of a disorder such as an autoimmune, inflammatory, or neurodegenerative disease, which can occur in multiple locations, is alleviated if the severity of the disease is decreased within at least one of multiple locations.

Compounds of the present invention can inhibit the cGAS/STING pathway and, accordingly, in one aspect of the invention, certain compounds disclosed herein are candidates for treating, or preventing certain conditions and diseases. The present invention provides methods for treating conditions and diseases wherein the course of the condition or disease can be influenced by the cGAS/STING pathway. The method includes administering to a subject in need of such treatment, a therapeutically effective amount of a compound of the present invention, or a pharmaceutically acceptable salt, metabolite, solvate, hydrate, or stereoisomer thereof.

The present invention provides a method of inhibiting the cGAS/STING pathway in a cell, comprising contacting the cell with one or more compounds or compositions of the present invention.

The present invention also provides a method of treating a cGAS/STING pathway-mediated condition, comprising administering to a patient in need thereof an effective amount of one or more compounds or compositions of the present invention. In some embodiments, the cGAS/STING pathway-mediated condition is an autoimmune, inflammatory, or neurodegenerative condition or cancer (see Rayburn, E. R et al., Mol Cell Pharmacol. 2009; 1(1): 29-43 and Urbanska, A. M, et al., Cell Biochem Biophys. 2015 July; 72(3):757-69).

The present invention also provides a method of inhibiting type I interferon production mediated by the cGAS/STING pathway comprising: administering to the subject in need thereof a therapeutically effective amount of one or more compounds or compositions of the present invention. The cGAS/STING pathway of cytosolic DNA sensing as that phrase is used herein comprises the following proteins: SAMHDI, DNase II, STAT1, STAT2, TREX1, ENPP1, cGAS. STING, IRF3, IRF7, TBK1, IKK, and NF-κB. Such a method may be practiced in vitro, in a cell, or in an organism (e.g., in a human).

The present invention also provides a method of treating a type I interferon-mediated disease in a subject, comprising: administering to the subject in need thereof a therapeutically effective amount of one or more compounds or compositions of the present invention.

The present invention also provides a method of inhibiting cytokine production in a cell, comprising: contacting the cell with one or more compounds or compositions of the present invention.

The present invention also provides a method of treating a cytokine-mediated disease in a subject, comprising: administering to the subject in need thereof a therapeutically effective amount of one or more compounds or compositions of the present invention.

The present invention provides a method of treating an autoimmune disease in a subject, comprising administering to the subject in need thereof a therapeutically effective amount of one or more compounds or compositions of the present invention. In some embodiments, the autoimmune disease can be a type I interferonopathy (e.g., Aicardi-Goutieres Syndrome, Sjögren's syndrome, Singleton-Merten Syndrome, proteasome-associated autoinflammatory syndrome, SAVI (STING-associated vasculopathy with onset in infancy), CANDLE syndrome, chilblain lupus erythematosus, systemic lupus erythematosus, spondyloenchondrodysplasia), rheumatoid arthritis, juvenile rheumatoid arthritis, idiopathic thrombocytopenic purpura, autoimmune myocarditis, thrombotic thrombocytopenic purpura, autoimmune thrombocytopenia, psoriasis, Type 1 diabetes, or Type 2 diabetes.

The present invention provides a method of treating an inflammatory disease in a subject, comprising administering to the subject in need thereof a therapeutically effective amount of one or more compounds or compositions of the present invention. For example, the inflammatory disease can be atherosclerosis, dermatomyositis, SIRS, sepsis, septic shock, celiac disease, interstitial cystitis, transplant rejection, inflammatory bowel disease (ulcerative colitis, Crohn's disease), age-related macular degeneration, IgA nephropathy, glomerulonephritis, vasculitis, polymyositis, or Wegener's disease.

The present invention further provides a method of treating neurodegenerative diseases in a subject, comprising administering to the subject in need thereof a therapeutically effective amount of one or more compounds or compositions of the present invention. For example, the neurodegenerative disease can be Alzheimer's disease, Parkinson's disease, multiple sclerosis, IgM polyneuropathies, or myasthenia gravis.

The compounds of the invention can also be use in combination with additional agents for the treatment of autoimmune and inflammatory diseases. Janus Kinase inhibitors (Jak inhibitors) including a Jak1. Jak2, Jak3 or Tyk2 inhibitor, or compound that inhibits any combination thereof, including Jak1/2 inhibitors. Jak inhibitors can block cytokine-mediated signaling via the JAK-STAT pathway, and have been developed for the treatment of a variety of inflammatory and autoimmune diseases. For example, tofacitinib is an approved Jak1 and Jak3 inhibitor used for the treatment of rheumatoid arthritis, psoriatic arthritis and ulcerative colitis; baricitinib is a Jak1 and Jak2 inhibitor approved in Europe, and used in the treatment of rheumatoid arthritis; filgotinib is a Jak1 inhibitor being developed for the treatment of rheumatoid arthritis and Crohn's disease.

The present invention provides a method of treating a disease in a subject, comprising administering to the subject in need thereof a therapeutically effective amount of a compound of the invention in combination with a Janus Kinase (Jak) inhibitor, including a Jak1, Jak2, Jak3 or Tyk2 inhibitor, or compound that inhibits any combination thereof. In some embodiments, the Jak inhibitor is a Jak1/2 inhibitor. In some embodiments, the Jak inhibitor is selected from the group consisting of ruxolitinib, tofacitinib, oclacitinib, baricitinib, filgotinib, gandotinib, itacitinib, lestaurtinib, momelotinib, pacritinib, upadacitinib, peficitinib, fedratinib, decemotinib, cerdulatinib, tasocitinib, PF-04965842, PF-06651600, PF-06700841, PF-06263276, BMS-986165, BMS-911543, AZD1480, AZD4205, AT9283, CHZ868, and TD-1473. In one embodiment, the disease is selected from the group consisting of SIRS, sepsis, septic shock, atherosclerosis, celiac disease, dermatomyositis, scleroderma, interstitial cystitis, transplant rejection (e.g. graft-versus-host disease), Aicardi-Goutieres Syndrome, Hutchison Guilford progeria syndrome, Singleton-Merten Syndrome, proteasome-associated autoinflammatory syndrome, SAVI, CANDLE syndrome, chilblain lupus erythematosus, systemic lupus erythematosus, rheumatoid arthritis, juvenile rheumatoid arthritis. Wegener's disease, inflammatory bowel disease (e.g. ulcerative colitis, Crohn's disease), idiopathic thrombocytopenic purpura, thrombotic thrombocytopenic purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjogren's syndrome. X-linked reticulate pigmentary disorder, polymyositis, spondyloenchondrodysplasia, age-related macular degeneration, Alzheimer's disease and Parkinson's disease. In some embodiments, compounds of the invention in combination with a Jak inhibitor are useful in treating Aicardi-Goutieres Syndrome, X-linked reticulate pigmentary disorder, dermatomyositis, systemic lupus erythematosus, rheumatoid arthritis, multiple sclerosis, or Type I or Type II diabetes.

The present invention further provides the use of one or more compounds or compositions of the present invention for inhibiting the cGAS/STING pathway in a cell.

The present invention further provides the use of one or more compounds or compositions of the present invention for the treatment of a cGAS/STING pathway-mediated condition.

The present invention further provides the use of one or more compounds or compositions of the present invention for inhibiting type I interferon production mediated by the cGAS/STING pathway in a cell.

The present invention further provides the use of one or more compounds or compositions of the present invention for the treatment of a type I interferon-mediated disease.

The present invention further provides the use of one or more compounds or compositions of the present invention for inhibiting cytokine production in a cell.

The present invention further provides the use of one or more compounds or compositions of the present invention for the treatment of a cGAS/STING pathway-mediated condition.

The present invention further provides the use of one or more compounds or compositions of the present invention for the treatment of an autoimmune disease. In some embodiments, the autoimmune disease can be a type I interferonopathy (e.g., Aicardi-Goutieres Syndrome, Sjögren's syndrome, Singleton-Merten Syndrome, proteasome-associated autoinflammatory syndrome, SAVI (STING-associated vasculopathy with onset in infancy). CANDLE syndrome, chilblain lupus erythematosus, systemic lupus erythematosus, spondyloenchondrodysplasia), rheumatoid arthritis, juvenile rheumatoid arthritis, idiopathic thrombocytopenic purpura, autoimmune myocarditis, thrombotic thrombocytopenic purpura, autoimmune thrombocytopenia, psoriasis, Type 1 diabetes, or Type 2 diabetes.

The present invention further provides the use of one or more compounds or compositions of the present invention for the treatment of an inflammatory disease. For example, the inflammatory disease can be atherosclerosis, dermatomyositis, SIRS, sepsis, septic shock, celiac disease, interstitial cystitis, transplant rejection, inflammatory bowel disease (ulcerative colitis, Crohn's disease), age-related macular degeneration, IgA nephropathy, glomerulonephritis, vasculitis, polymyositis, or Wegener's disease.

The present invention further provides the use of one or more compounds or compositions of the present invention for the treatment of a neurodegenerative disease. For example, the neurodegenerative disease can be Alzheimer's disease, Parkinson's disease, multiple sclerosis, IgM polyneuropathies, or myasthenia gravis.

The present invention further provides one or more compounds or compositions of the present invention for use in inhibiting the cGAS/STING pathway in a cell.

The present invention further provides one or more compounds or compositions of the present invention for use in the treatment of a cGAS/STING pathway-mediated condition.

The present invention further provides one or more compounds or compositions of the present invention for use in inhibiting type I interferon production mediated by the cGAS/STING pathway in a cell.

The present invention further provides one or more compounds or compositions of the present invention for use in the treatment of a type I interferon-mediated disease.

The present invention further provides one or more compounds or compositions of the present invention for use in inhibiting cytokine production in a cell.

The present invention further provides one or more compounds or compositions of the present invention for use in the treatment of a cGAS/STING pathway-mediated condition.

The present invention further provides one or more compounds or compositions of the present invention for use in the treatment of an autoimmune disease, such as those described herein.

The present invention further provides one or more compounds or compositions of the present invention for use in the treatment of an inflammatory disease, such as those described herein.

The present invention further provides one or more compounds or compositions of the present invention for use in the treatment of a neurodegenerative disease, such as those described herein.

Any of the one or more compounds or compositions for use described above may be for use in combination with a Janus Kinase (Jak) inhibitor, such as those described herein.

The present invention further provides the use of one or more compounds or compositions of the present invention in the manufacture of a medicament for inhibiting the cGAS/STING pathway in a cell.

The present invention further provides the use of one or more compounds or compositions of the present invention in the manufacture of a medicament for the treatment of a cGAS/STING pathway-mediated condition.

The present invention further provides the use of one or more compounds or compositions of the present invention in the manufacture of a medicament for inhibiting type I interferon production in a cell.

The present invention further provides the use of one or more compounds or compositions of the present invention in the manufacture of a medicament for the treatment of a type I interferon-mediated disease.

The present invention further provides the use of one or more compounds or compositions of the present invention in the manufacture of a medicament for inhibiting cytokine production in a cell.

The present invention further provides the use of one or more compounds or compositions of the present invention in the manufacture of a medicament for the treatment of a cytokine-mediated condition.

The present invention further provides the use of one or more compounds or compositions of the present invention in the manufacture of a medicament for the treatment of an autoimmune disease, such as those described herein.

The present invention further provides the use of one or more compounds or compositions of the present invention in the manufacture of a medicament for the treatment of an inflammatory disease, such as those described herein.

The present invention further provides the use of one or more compounds or compositions of the present invention in the manufacture of a medicament for the treatment of a neurodegenerative disease, such as those described herein.

The present invention further provides the use of one or more compounds or compositions of the present invention in combination with a Janus Kinase (Jak) inhibitor, including a Jak1, Jak2, Jak3 or Tyk2 inhibitor, or compound that inhibits any combination thereof, in the manufacture of a medicament for the treatment of a disease selected from the group consisting of SIRS, sepsis, septic shock, atherosclerosis, celiac disease, dermatomyositis, scleroderma, interstitial cystitis, transplant rejection (e.g. graft-versus-host disease), Aicardi-Goutieres Syndrome, Hutchison Guilford progeria syndrome, Singleton-Merten Syndrome, proteasome-associated autoinflammatory syndrome, SAVI, CANDLE syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenic purpura, thrombotic thrombocytopenic purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjogren's syndrome, X-linked reticulate pigmentary disorder, polymyositis, spondyloenchondrodysplasia, age-related macular degeneration, Alzheimer's disease and Parkinson's disease. In some embodiments, the Jak inhibitor is a Jak1/2 inhibitor. In some embodiments, the Jak inhibitor is selected from the group consisting of ruxolitinib, tofacitinib, oclacitinib, baricitinib, filgotinib, gandotinib, itacitinib, lestaurtinib, momelotinib, pacritinib, upadacitinib, peficitinib, fedratinib, decemotinib, cerdulatinib, tasocitinib, PF-04965842, PF-06651600, PF-06700841, PF-06263276, BMS-986165, BMS-911543, AZD1480, AZD4205, AT9283, CHZ868, and TD-1473.

cGAS inhibitory activity of any of the compounds disclosed herein can be determined by reacting the compound in a properly buffered environment with a DNA-activated cGAS in the presence of ATP and GTP. Antagonist activity can then be quantified by measuring the amount of ATP and/or GTP remaining after reaction is halted. Human cGAS sequence encoding amino acids 155-522 (DAAPGASKLRAVLEKLKLSRDDISTAAGMVKGVVDHLLLRLKCDSAFRGVGLLNT GSYYEHVKISAPNEFDVMFKLEVPRIQLEEYSNTRAYYFVKFKRNPKENPLSQFLEGE ILSASKMLSKFRKIIKEEINDIKDTDVIMKRKRGGSPAVTLLISEKISVDITLALESKSS WPASTQEGLRIQNWLSAKVRKQLRLKPFYLVPKHAKEGNGFQEETWRLSFSHIEKEI LNNHGKSKTCCENKEEKCCRKDCLKLMKYLLEQLKERFKDKKHLDKFSSYHVKTA FFHVCTQNPQDSQWDRKDLGLCFDNCVTYFLQCLRTEKLENYFIPEFNLFSSNLIDKR SKEFLTKQIEYERNNEFPVFDEF, SEQ ID No. 1) can be cloned into an expression plasmid to create a construct containing codes for the appropriate proteins and tags (e.g. hexahistidine tag, maltose binding protein fusion, and a cleavable linker) preceding the cGAS sequence. The protein can then be expressed and purified using standard techniques.

The cGAS/STING pathway inhibitory activity of any of the compounds disclosed herein can also be determined by measuring changes in the type I interferon signature resulting from administration of the compound(s).

Potential cGAS antagonists, e.g., the pyrazolopyrimidinone compounds disclosed herein, can be made to interact, in a properly buffered environment, with a DNA-activated cGAS in the presence of ATP and GTP. Antagonist activity can then be quantified by measuring the amount of ATP and/or GTP remaining after reaction is halted.

A cellular assay can be used to assess the compounds of the invention for their ability to inhibit the cGAS/STING pathway. Cells that express a luciferase-based reporter that is linked to IRF-3 activation are used to determine response as a function of compound concentration. Such an assay is described in Vincent et al., Nature Communications 2017, 8(1):750, doi: 10.1038/s41467-017-00833-9.

A cellular assay can be used to asses the compounds of the invention for their ability to inhibit cytokine production. Bone marrow macrophages harvested from mice can be used to determine response as a function of compound concentration.

### Pharmaceutical Compositions

The present invention also provides pharmaceutical compositions comprising a compound of any Formula disclosed herein in combination with at least one pharmaceutically acceptable excipient or carrier.

A “pharmaceutical composition” is a formulation containing the compounds of the present invention in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the invention can be administered to a subject in many of the well-known methods currently used for chemotherapeutic treatment. The dose chosen should be sufficient to constitute effective treatment but not so high as to cause unacceptable side effects. The state of the disease condition and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present invention may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In general, a compound of the application will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. Therapeutic amounts or doses will also vary depending on route of administration, as well as the possibility of co-usage with other agents.

Upon improvement of a subject's condition, a maintenance dose of a compound, composition or combination of this application may be administered, if necessary. Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present application will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The term “pharmaceutical combination” as used herein means a product that results from the mixing or combining of more than one active ingredient and includes both fixed and non-fixed combinations of the active ingredients. The term “fixed combination” means that the active ingredients, e.g., a compound of the application and a co-agent, are both administered to a patient simultaneously in the form of a single entity or dosage. The term “non-fixed combination” means that the active ingredients, e.g., a compound of the application and a co-agent, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It can be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

In therapeutic applications, the dosages of the pharmaceutical compositions used in accordance with the invention vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the progression of the autoimmune, neurodegenerative, or inflammatory disease. Dosages can be in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. As used herein, the term “dosage effective manner” refers to amount of an active compound to produce the desired biological effect in a subject or cell.

The pharmaceutical compositions can be included in a container, pack, or dispenser together with instructions for administration.

The compounds of the present invention are capable of further forming salts. All of these forms are also contemplated within the scope of the claimed invention.

As used herein, “pharmaceutically acceptable salts” refer to derivatives of the compounds of the present invention wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, bisulfate, bitartric, boric, bromic, butyric, calcium, calcium edetic, camsylate, carbonic, chloric, citric, clavularic, dihydrochloric, edetic, ethane disulfonic, 1,2-ethane sulfonic, estolate, esylate, fumaric, glucoheptonic, gluconic, glutamic, glycolic, glycollyarsanilic, hexafluorophosphoric, hexylresorcinic, hydrabamic, hydrobromic, hydrochloric, hydroiodic, hydroxymaleic, hydroxynaphthoic, iodic, isethionic, lactic, lactobionic, lauryl sulfonic, maleic, malic, mandelic, methane sulfonic, methylbromic, methylnitric, napsylic, nitric, N-methylglucamine ammonium salt, 3-hydroxy-2-naphthoic, oleic, oxalic, pamoic, pantothenic, phenylacetic, phosphoric, polygalacturonic, propionic, salicyclic, stearic, subacetic, succinic, sulfamic, sulfanilic, sulfuric, sulfosalicylic, suramic, tannic, tartaric, toluene sulfonic, tosyl, triethiodic, trifluoroacetic, and valeric and the commonly occurring amine acids, e.g., glycine, alanine, phenylalanine, arginine, etc.

In addition to pharmaceutically acceptable salts, the compounds and pharmaceutically acceptable salts as disclosed herein can be “pharmaceutically acceptable solvates”, or where the solvent is water, “pharmaceutically acceptable hydrates”. For example, pharmaceutically acceptable solvates may be formed wherein solvent molecules are incorporated into the crystalline lattice during crystallization. Solvates may involve nonaqueous solvents such as ethanol, isopropanol, dimethyl sulfoxide, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates or hydrates

When a disclosed compound or its salt is named or depicted by structure, it is to be understood that the compound or salt, including solvates or hydrates thereof, may exist in crystalline forms, non-crystalline forms or a mixture thereof. The compound or salt, or solvates or hydrates thereof, may also exhibit polymorphism (i.e. the capacity to occur in different crystalline forms). These different crystalline forms are typically known as “polymorphs.” It is to be understood that when named or depicted by structure, the disclosed compound, or solvates or hydrates thereof, also include all polymorphs thereof. Polymorphs have the same chemical composition but differ in packing, geometrical arrangement, and other descriptive properties of the crystalline solid state. Polymorphs may have different physical properties such as density, shape, hardness, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used during the crystallization or recrystallization of the compounds described herein.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present invention also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1, e.g., 3:1, 2:1, 1:2, or 1:3.

It should be understood that all references to pharmaceutically acceptable salts include solvent addition forms (solvates) or crystal forms (polymorphs) as defined herein, of the same salt.

The compounds of the present invention can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds of the present invention can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present invention can be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a subject. Prodrugs in the present invention are prepared by modifying functional groups present in the compound in such a way that the modifications are cleaved, either in routine manipulation or in vivo, to the parent compound. Prodrugs include compounds of the present invention wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the invention, and the like. See Bundegaard, H., Design of Prodrugs, p 1-92, Elesevier, New York-Oxford (1985).

The compounds, or pharmaceutically acceptable salts, esters or prodrugs thereof, are administered by a route selected from the group consisting of enterally, orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the invention can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

In the synthetic schemes described herein, compounds may be drawn with one particular configuration for simplicity. Such particular configurations are not to be construed as limiting the invention to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

The present invention provides a kit comprising a compound capable of inhibiting the cGAS/STING pathway selected from one or more compounds of the present invention, or a pharmaceutically acceptable salt, pharmaceutically acceptable solvate or pharmaceutically acceptable hydrate thereof, and instructions for use in treating an autoimmune disease. In some embodiments, the autoimmune disease can be a type I interferonopathy (e.g., Aicardi-Goutieres Syndrome, Sjögren's syndrome, Singleton-Merten Syndrome, proteasome-associated autoinflammatory syndrome, SAVI (STING-associated vasculopathy with onset in infancy). CANDLE syndrome, chilblain lupus erythematosus, systemic lupus erythematosus, spondyloenchondrodysplasia), rheumatoid arthritis, juvenile rheumatoid arthritis, idiopathic thrombocytopenic purpura, autoimmune myocarditis, thrombotic thrombocytopenic purpura, autoimmune thrombocytopenia, psoriasis, Type 1 diabetes, or Type 2 diabetes.

The present invention provides a kit comprising a compound capable of inhibiting the cGAS/STING pathway selected from one or more compounds of the present invention, or a pharmaceutically acceptable salt, pharmaceutically acceptable solvate or pharmaceutically acceptable hydrate thereof, and instructions for use in treating an inflammatory disease. In some embodiments, the inflammatory disease can be atherosclerosis, dermatomyositis, SIRS, sepsis, septic shock, celiac disease, interstitial cystitis, transplant rejection, inflammatory bowel disease (ulcerative colitis, Crohn's disease), age-related macular degeneration. IgA nephropathy, glomerulonephritis, vasculitis, polymyositis, or Wegener's disease.

The present invention provides a kit comprising a compound capable of inhibiting the cGAS/STING pathway selected from one or more compounds of the present invention, or a pharmaceutically acceptable salt, pharmaceutically acceptable solvate or pharmaceutically acceptable hydrate thereof, and instructions for use in treating a neurodegenerative disease.

In some embodiments, the neurodegenerative disease can be Alzheimer's disease, Parkinson's disease, multiple sclerosis, IgM polyneuropathies, or myasthenia gravis.

The present invention provides a kit comprising a compound capable of inhibiting type I interferon production selected from one or more compounds of the present invention, or a pharmaceutically acceptable salt, pharmaceutically acceptable solvate or pharmaceutically acceptable hydrate thereof, and instructions for use in treating an autoimmune disease. In some embodiments, the autoimmune disease can be a type I interferonopathy (e.g., Aicardi-Goutieres Syndrome, Sjogren's syndrome, Singleton-Merten Syndrome, proteasome-associated autoinflammatory syndrome, SAVI (STING-associated vasculopathy with onset in infancy), CANDLE syndrome, chilblain lupus erythematosus, systemic lupus erythematosus, spondyloenchondrodysplasia), rheumatoid arthritis, juvenile rheumatoid arthritis, idiopathic thrombocytopenic purpura, autoimmune myocarditis, thrombotic thrombocytopenic purpura, autoimmune thrombocytopenia, psoriasis, Type 1 diabetes, or Type 2 diabetes.

The present invention provides a kit comprising a compound capable of inhibiting type I interferon production selected from one or more compounds of the present invention, or a pharmaceutically acceptable salt, pharmaceutically acceptable solvate or pharmaceutically acceptable hydrate thereof, and instructions for use in treating an inflammatory disease. In some embodiments, the inflammatory disease can be atherosclerosis, dermatomyositis, SIRS, sepsis, septic shock, celiac disease, interstitial cystitis, transplant rejection, inflammatory bowel disease (ulcerative colitis, Crohn's disease); age-related macular degeneration, IgA nephropathy, glomerulonephritis, vasculitis, polymyositis, or Wegener's disease.

The present invention provides a kit comprising a compound capable of inhibiting type I interferon production selected from one or more compounds of the present invention, or a pharmaceutically acceptable salt, pharmaceutically acceptable solvate or pharmaceutically acceptable hydrate thereof, and instructions for use in treating a neurodegenerative disease. In some embodiments, the neurodegenerative disease can be Alzheimer's disease, Parkinson's disease, multiple sclerosis, IgM polyneuropathies, or myasthenia gravis.

The present invention provides a kit comprising a compound capable of inhibiting cytokine production selected from one or more compounds of the present invention, or a pharmaceutically acceptable salt, pharmaceutically acceptable solvate or pharmaceutically acceptable hydrate thereof, and instructions for use in treating an autoimmune disease. In some embodiments, the autoimmune disease can be a type I interferonopathy (e.g., Aicardi-Goutieres Syndrome, Sjögren's syndrome. Singleton-Merten Syndrome, proteasome-associated autoinflammatory syndrome, SAVI (STING-associated vasculopathy with onset in infancy). CANDLE syndrome, chilblain lupus erythematosus, systemic lupus erythematosus, spondyloenchondrodysplasia), rheumatoid arthritis, juvenile rheumatoid arthritis, idiopathic thrombocytopenic purpura, autoimmune myocarditis, thrombotic thrombocytopenic purpura, autoimmune thrombocytopenia, psoriasis, Type 1 diabetes, or Type 2 diabetes.

The present invention provides a kit comprising a compound capable of inhibiting cytokine production selected from one or more compounds of the present invention, or a pharmaceutically acceptable salt, pharmaceutically acceptable solvate or pharmaceutically acceptable hydrate thereof, and instructions for use in treating an inflammatory disease. In some embodiments, the inflammatory disease can be atherosclerosis, dermatomyositis, SIRS, sepsis, septic shock, celiac disease, interstitial cystitis, transplant rejection, inflammatory bowel disease (ulcerative colitis, Crohn's disease), age-related macular degeneration, IgA nephropathy, glomerulonephritis, vasculitis, polymyositis, or Wegener's disease.

The present invention provides a kit comprising a compound capable of inhibiting cytokine production selected from one or more compounds of the present invention, or a pharmaceutically acceptable salt, pharmaceutically acceptable solvate or pharmaceutically acceptable hydrate thereof, and instructions for use in treating a neurodegenerative disease. In some embodiments, the neurodegenerative disease can be Alzheimer's disease, Parkinson's disease, multiple sclerosis, IgM polyneuropathies, or myasthenia gravis.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXEMPLARY EMBODIMENTS

### Embodiment I-1

A compound of Formula (I):

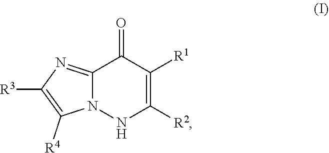

or a pharmaceutically acceptable salt, pharmaceutically acceptable solvate or pharmaceutically acceptable hydrate thereof, wherein

R¹ is Q¹-T¹-(X¹)ₙ;

Q¹ is a bond or C₁₋₃alkylene, wherein the C₁₋₃alkylene group is optionally substituted with one or more substituents independently selected from the group consisting of halo, C₁₋₆alkyl, C₂₋₆alkenyl, C₂₋₆alkynyl, cyano, C₁₋₆haloalkyl, —OR^{w2}, and —NR^{w2}R^{x2};

T¹ is H, halo, cyano, C₃₋₈cycloalkyl, C₆₋₁₀aryl, 3- to 12-membered heterocycloalkyl, 5- to 10-membered heteroaryl, —C(═O)C₀₋₃alkylene-C₃₋₈cycloalkyl, —C(═O)—C₀₋₃alkylene-C₆₋₁₀aryl, —C(═O)—C₀₋₃alkylene-3 to 12-membered heterocycloalkyl, —C(═O)—C₀₋₃alkylene-5 to 10-membered heteroaryl, —C(═O)R^{a}, —C(═O)OR^{a}, —NR^{a}R^{b}. —S(═O)₂R^{a}, —NR^{a}C(═O)R^{a}, —NR^{a}C(═O)NR^{a}R^{b}, —NR^{a}C(═O)OR^{a}, —NR^{a}S(═O)₂R^{a}, —C(═O)NR^{a}S(═O)₂R^{a}, —NR^{a}S(═O)₂NR^{a}R^{b}, —C(═O)NR^{a}R^{b}, or —S(═O)₂NR^{a}R^{b};

each X¹ is independently selected from the group consisting of halo, cyano, oxo, C₀₋₃alkylene-C(═O)R^{c}, C₀₋₃alkylene-OR^{c}, C₀₋₃alkylene-C(═O)OR^{c}, C₀₋₃alkylene-OC(═O)R, C₀₋₃alkylene-NR^{c}R^{d}, C₀₋₃alkylene-N⁺R^{c}R^{d}R^{d′}, C₀₋₃alkylene-S(═O)ₘR^{c}, C₀₋₃alkylene-NR^{c}C(═O)R^{c}, C₀₋₃alkylene-NR^{c}C(═O)NR^{c}R^{d}, C₀₋₃alkylene-OC(═O)NR^{c}R^{d}, C₀₋₃alkylene-NR^{c}C(═O)OR^{c}, C₀₋₃alkylene-NR^{c}S(═O)₂R^{c}, C₀₋₃alkylene-C(═O)NR^{c}S(═O)₂R^{c}, C₀₋₃alkylene-NR^{c}S(═O)₂NR^{c}R^{d}, C₀₋₃alkylene-C(═O)NR^{c}R^{d}, C₀₋₃alkylene-S(═O)₂NR^{c}R^{d}, C₀₋₃alkylene-C(═NR^{c})NR^{c}R^{d}, C₀₋₃alkylene-NR^{c}C(═NR^{c})NR^{c}R^{d}, and R^{S1}, in which R^{s1} is C₁-6alkyl, C₂₋₆alkenyl, C₂₋₆alkynyl, C₀₋₃alkylene-C₃₋₈cycloalkyl, C₀₋₃alkylene-C₆₋₁₀ aryl, C₀₋₃alkylene-3- to 12-membered heterocycloalkyl, or C₀₋₃alkylene-5- to 10-membered heteroaryl;

and each R^{S1} is optionally substituted with one or more substituents independently selected from the group consisting of halo, cyano, nitro, oxo, C₁₋₆alkyl, C₂₋₆alkenyl, C₂₋₆alkynyl, C₁₋₆haloalkyl, C₀₋₃alkylene-NR^{e}R^{f}, C₀₋₃alkylene-OR^{e}, C₀₋₃alkylene-NR^{e}C(═O)R^{e}, C₀₋₃alkylene-NR^{e}C(═O)OR^{e}, C₀₋₃alkylene-NR^{e}C(═O)NR^{e}R^{f}, C₀₋₃alkylene-OC(═O)R^{e}, C₀₋₃alkylene-C(═O)OR^{e}, C₀₋₃alkylene-C(═O)NR^{e}R^{f}, C₀₋₃alkylene-C(═O)R, C₀₋₃alkylene-S(═O)ₘR^{e}, C₀₋₃alkylene-S(═O)₂NR^{e}R^{f}, C₀₋₃alkylene-NR^{e}S(═O)₂R^{e}, C₀₋₃alkylene-C(═O)NR^{e}S(═O)₂R^{e}, C₀₋₃alkylene-NR^{e}S(═O)₂NR^{e}R^{f}, and R^{S2}, in which R^{S2} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl,

and each R^{S2} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w}, and —NR^{w}R^{x};

R² is Q²-T²-(X²)ₚ;

Q² is a bond or C₁₋₆alkylene, wherein the C₁₋₃alkylene group is optionally substituted with one or more substituents independently selected from the group consisting of halo, C₁₋₆alkyl, C₂₋₆alkenyl, C₂₋₆alkynyl, cyano, C₁₋₆haloalkyl, —OR^{w3}, and —NR^{w3}R^{x3};

T² is H, halo, cyano, C₁₋₆alkyl, C₂₋₆alkenyl, C₂₋₆alkynyl, C₃₋₈cycloalkyl, C₆₋₁₀aryl, 3- to 12-membered heterocycloalkyl, 5- to 10-membered heteroaryl, —C(═O)—C₀₋₃alkylene-C₃₋₈cycloalkyl, —C(═O)—C₀₋₃alkylene-C₆₋₁₀aryl, —C(═O)—C₀₋₃alkylene-3- to 12-membered heterocycloalkyl, —C(═O)—C₀₋₃alkylene-5- to 10-membered heteroaryl, —OR^{z}, —S(═O)ₘR^{k}, —P(═O)R^{kk}R^{mm}, —NR^{k}R^{m}, —C(═O)OR^{k}, or —C(═O)NR^{k}R^{m};

each X² is independently selected from the group consisting of halo, cyano, oxo, C₀₋₃alkylene-ORⁿ, C₀₋₃alkylene-S(═O)ₘRⁿ, C₀₋₃alkylene-NRⁿR^{o}, C₀₋₃alkylene-C(═O)NRⁿR^{o}, C₀₋₃alkylene-C(═O)ORⁿ, and R^{S3}, in which R^{S3} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and R^{S3} is optionally substituted with one or more substituents independently selected from the group consisting of halo, oxo, cyano, C₀₋₃alkylene-OR^{p}, C₀₋₃alkylene-S(═O)ₘR^{p}, C₀₋₃alkylene-NR^{p}R^{a}, C₀₋₃alkylene-C(═O)NR^{p}R^{q}, C₀₋₃alkylene-C₀₋₃alkylene-C(═O)OR, C₁₋₆alkyl, C₂₋₆alkenyl, C₂₋₆alkynyl, C₁₋₆haloalkyl, and R^{S4} in which R^{S4} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S4} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w4}, and —NR^{w4}R^{x4};

R³ is halo, C₁₋₃alkyl, C₁₋₃haloalkyl, C₂₋₃alkenyl, C₂₋₆alkynyl, C₃₋₈cycloalkyl, —CN, —OR^{r}, —C(═O)R^{r}, —S(═O)ₘR^{r}, NR^{r}R^{t}, or —C(═O)OR^{r}, wherein C₁₋₆alkyl, C₂₋₃alkenyl and C₂₋₃alkynyl are optionally substituted with one C₃₋₆cycloalkyl;

R⁴ is C₁₋₆alkyl, C₁₋₆haloalkyl, S(═O)ₘR^{u}, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl, wherein C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl are optionally substituted with one or more substituents independently selected from the group consisting of halo, oxo, C₁₋₆alkyl, C₂₋₆alkenyl, C₂₋₆alkynyl, cyano, C₁₋₆haloalkyl, OR^{w5}, and NR^{w5}R^{x5};

each of R^{a} and R^{b}, independently, is H or R^{S5}, in which R^{S5} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and R^{S5} is optionally substituted with one or more substituents independently selected from the group consisting of halo, cyano, oxo, C₁₋₆haloalkyl, C₀₋₃alkylene-OR^{c2}, C₀₋₃alkylene-C(═O)R^{c2}, C₀₋₃alkylene-C(═O)OR^{2′}, C₀₋₃alkylene-OC(═O)R², C₀₋₃alkylene-C(═O)NR^{c2}R^{d2}, C₀₋₃alkylene-S(═O)ₘR^{d}, C₀₋₃alkylene-S(═O)₂NR^{c2}R^{d2}, C₀₋₃alkylene-NR^{c2}R^{d2}, C₀₋₃alkylene-NR^{c2}C(═O)R^{c2}, C₀₋₃alkylene-NR^{c2}C(═O)OR^{c2}, C₀₋₃alkylene-NR^{c2}C(═O)NR^{c2}R^{d2}, C₀₋₃alkylene-NR^{c2}S(═O)₂R^{c2}, C₀₋₃alkylene-C(═O)NR^{c2}S(═O)₂R^{c2}, C₀₋₃alkylene-NR^{c2}S(═O)₂NR^{c2}R^{d2}, C₀₋₃alkylene-N(S(═O)₂R^{c2})₂, and R^{S1}, in which R^{S6} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S6} is optionally substituted with one or more substituents independently selected from the group consisting of halo, cyano, oxo, C₁₋₆alkyl, C₂₋₆alkenyl, C₂₋₆alkynyl, C₁₋₆haloalkyl, C₀₋₃alkylene-NR^{e2}R^{f2}, C₀₋₃alkylene-OR^{e2}, C₀₋₃alkylene-NR^{e2}C(═O)R^{e2}, C₀₋₃alkylene-NR^{e2}C(═O)OR^{e2}, C₀₋₃alkylene-NR^{e2}C(═O)NR^{e2}R^{f2}, C₀₋₃alkylene-OC(═O)R^{e2}, C₀₋₃alkylene-C(═O)OR^{e2}, C₀₋₃alkylene-C(═O)NR^{e2}R^{f2}, C₀₋₃alkylene-C(═O)R^{e}, C₀₋₃alkylene-S(═O)ₘR^{e2}, C₀₋₃alkylene-S(═O)₂NR^{e2}R^{f2}, C₀₋₃alkylene-NR^{e2}S(═O)₂R^{e2}, C₀₋₃alkylene-C(═O)NR^{e2}S(═O)₂R^{e2}, C₀₋₃alkylene-NR^{e2}S(═O)₂NR^{e2}R^{f2}, and R^{S7}, in which R^{S7} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S1} is optionally substituted with one or more substituents independently selected from the group consisting of halo, oxo, C₆₋₁₀alkyl, C₂₋₆alkenyl, C₂₋₆alkynyl, cyano, C₁₋₆haloalkyl, —OR^{w6}, and —NR^{w6}R^{x6};

each of R, R^{c2}, R^{d}, R^{d′}, and R^{d2}, independently, is H or R^{S8}, in which R^{S8} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl; and each R^{S5} is optionally substituted with one or more substituents independently selected from the group consisting of halo, cyano, oxo, C₁₋₆alkyl, C₂₋₆alkenyl, C₂₋₆alkynyl, C₁₋₆haloalkyl, C₀₋₃alkylene-NR^{e3}R^{f3}, C₀₋₃alkylene-OR^{e3}, C₀₋₃alkylene-C(═O)OR^{e3}, C₀₋₃alkylene-C(═O)NR^{e3}R^{f3}, C₀₋₃alkylene-C(═O)R^{e3}, C₀₋₃alkylene-S(═O)ₘR^{e3}, C₀₋₃alkylene-S(═O)₂NR^{e3}R^{f3}, C₀₋₃alkylene-NR^{e3}C(═O)R^{e3}, C₀₋₃alkylene-NR^{e3}S(═O)ₘR^{e3}, and R^{S9}, in which R^{S9} is C₀₋₃alkylene-C₃₋₈cycloalkyl, C₀₋₃alkylene-C₆₋₁₀aryl, C₀₋₃alkylene-3- to 12-membered heterocycloalkyl, C₀₋₃alkylene-5- to 10-membered heteroaryl;

and each R^{S9} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w7}, and —NR^{w7}R^{x7};

each of R^{e}, R^{e2}, R^{e3}, R^{f}, R^{f2}, and R^{f3}, independently, is H or R^{S10}, in which R^{S10} is C₁₋₆alkyl, C₂₋₆alkenyl, C₁₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S10} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w8}, and —NR^{w8}R^{x8};

each of R^{kk} and R^{mm}, is independently selected from the group consisting of R^{k}, —OR^{k}, and —NR^{k}R^{m};

each of R^{k}, and R^{m}, independently, is H or R¹, in which R¹ is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{z} is optionally substituted with one or more substituents independently selected from the group consisting of halo, cyano, oxo, C₁₋₆alkyl, C₂₋₆alkenyl, C₂₋₆alkynyl, C₁₋₆haloalkyl, C₀₋₃alkylene-NRⁿ²R^{o2}, C₀₋₃alkylene-ORⁿ², C₀₋₃alkylene-C(═O)ORⁿ², C₀₋₃alkylene-C(═O)NRⁿ²R^{o2}, C₀₋₃alkylene-C(═O)Rⁿ², C₀₋₃alkylene-S(═O)ₘRⁿ², C₀₋₃alkylene-S(═O)₂NRⁿ²R^{o2}, and R^{S11}, in which R^{S11} is C₀₋₃alkylene-C₃₋₈cycloalkyl, C₀₋₃alkylene-C₆₋₁₀aryl, C₀₋₃alkylene-3- to 12-membered heterocycloalkyl, C₀₋₃alkylene-5- to 10-membered heteroaryl;

and each R^{S11} is optionally substituted with one or more substituents independently selected from the group consisting of halo, oxo, cyano, C₀₋₃alkylene-OR^{p2}, C₀₋₃alkylene-S(═O)ₘR^{p2}, C₀₋₃alkylene-NR^{p2}R^{q2}, C₀₋₃alkylene-C(═O)NR^{p2}R^{q2}, C₀₋₃alkylene-C₀₋₃alkylene-C(═O)OR^{p2}, C₁₋₆alkyl, C₂₋₆alkenyl, C₂₋₆alkynyl, C₁₋₆haloalkyl, and R^{S12}, in which R^{S12} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each R^{S12} is optionally substituted with one or more substituents independently selected from the group consisting of halo, oxo, C₆₋₁₀alkyl, C₂₋₆alkenyl, C₂₋₆alkynyl, cyano, C₁₋₆haloalkyl, —OR^{w9}, and —NR^{w9}R^{x9};

each of Rⁿ, Rⁿ², R^{o}, and R^{o2}, independently, is H or R^{S13}, in which R^{S13} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each R^{S13} is optionally substituted with one or more substituents independently selected from the group consisting of halo, oxo, cyano, C₀₋₃alkylene-OR³, C₀₋₃alkylene-S(═O)ₘR^{p3}, C₀₋₃alkylene-NR^{p3}R^{q3}, C₀₋₃alkylene-C(═O)NR^{p3}R^{q3}, C₀₋₃alkylene-C(═O)OR^{p3}, C₁₋₆alkyl, C₂₋₆alkenyl, C₂₋₆alkynyl, C₁₋₆haloalkyl, and R^{S14}, in which R^{S14} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each R^{S14} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w10}, and —NR^{w10}R^{x10};

each of R^{p}, R^{p2}, R^{p3}, R^{q}, R^{q2}, and R^{q3}; independently, is H or R^{S15}, in which R^{S15} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

each R^{S15} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w11}, and —NR^{w11}R^{x11};

each of R^{r}, R^{t}, and R^{u}, independently, is H or R^{S16}, in which R^{S16} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;

and each R^{S16} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —C(═O)OR^{w12}, —OR^{w12}, and —NR^{w12}R^{x12};

each R^{w}, R^{w2}, R^{w3}, R^{w4}, R^{w5}, R^{w6}, R^{w7}, R^{w8}, R^{w9}, R^{w10}, R^{w11}, R^{w12}, R^{x}, R^{x2}, R^{x3}, R^{x4}, R^{x5}, R^{x6}, R^{x7}, R^{x8}, R^{x9}, R^{x10}, R^{x11}, and R^{x12}, independently, is H, C₁₋₃alkyl, C₂₋₆alkenyl, C₂₋₆alkynyl or C₁₋₆haloalkyl;

each of n and p independently is 0, 1, 2, 3, 4, or 5, wherein when T¹ is H, n is 0, and when T² is H, p is 0; and

m is 0, 1, or 2;

with the proviso that the compound is not

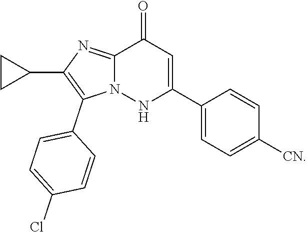 [SMILES: N#Cc1ccc(-c2cc(=O)c3nc(C4CC4)c(-c4ccc(Cl)cc4)n3[nH]2)cc1]

### Embodiment I-2

The compound of Embodiment I-1, wherein Q¹ is a bond and T¹ is C₆₋₁₀aryl, 3- to 12-membered heterocycloalkyl, or 5- to 10-membered heteroaryl, and n is 0, 1, 2, 3 or 4.

### Embodiment I-3

The compound of Embodiment I-1, wherein Q¹ is a bond or —CH₂— and T¹ is C₃₋₈cycloalkyl, C₆₋₁₀aryl, 3- to 12-membered heterocycloalkyl, 5- to 10-membered heteroaryl, or —C(═O)NR^{a}R^{b}.

### Embodiment I-4

The compound of Embodiment I-1, wherein Q¹ is a bond or —CH₂—. T¹ is —C(═O)NR^{a}R^{b} and n is 0.

### Embodiment I-5

The compound of any one of Embodiments I-1 to I-3, wherein T¹ is 9- or 10-membered bicyclic heteroaryl.

### Embodiment I-6

The compound of any one of Embodiments I-1, I-3 and I-4, wherein one of R^{a} and R^{b} is H or methyl.

### Embodiment I-7

The compound of any one of Embodiments I-1, 1-3 and I-4, wherein one of R^{a} and R^{b} independently is pyridinyl, pyrazinyl, pyridazinyl, pyrimidinyl, indolyl, indazolyl, benzimidazolyl, imidazopyridinyl, quinolinyl, isoquinolinyl, quinazolinyl, oxazolyl, isoxazolyl, thiazolyl, isothiazolyl, benzoxazolyl, oxadiazolyl, triazolyl, imidazolyl, furan, or thiophenyl, and the other is hydrogen or methyl.

### Embodiment I-8

The compound of any one of Embodiments I-1 to I-7, wherein R² is Q²-T²-(X²)ₚ, Q² is a bond, T² is H, halo, cyano, C₁₋₆alkyl, C₁₋₆haloalkyl, C₂₋₆alkenyl, or C₂₋₆alkynyl, and each X² independently is halo or —OC₁₋₆alkyl.

### Embodiment I-9

The compound of any one of Embodiments I-1 to I-7, wherein R² is H, cyano, methyl or methoxymethyl.

### Embodiment I-10

The compound of any one of Embodiments I-1 to I-9, wherein R³ is C₁₋₃alkyl, C₁₋₃haloalkyl, —CN, —S(═O)₂C₁₋₃alkyl or —C(═O)OC₁₋₃alkyl.

### Embodiment I-11

The compound of any one of Embodiments I-1 to I-9, wherein R³ is —CN, C₁₋₆alkyl, C₁₋₃haloalkyl or —C(═O)OC₁₋₃alkyl.

### Embodiment I-12

The compound of any one of Embodiments I-1 to I-9, wherein R³ is —CN or —CF₃.

### Embodiment I-13

The compound of any one of Embodiments I-1 to I-12, wherein R⁴ is C₁₋₃alkyl, C₁₋₃haloalkyl, —S(═O)₂C₁₋₃alkyl, C₃₋₈cycloalkyl, C₆₋₁₀aryl, 3- to 12-membered heterocycloalkyl, or 5- to 10-membered heteroaryl, wherein C₃₋₈cycloalkyl, C₆₋₁₀aryl, 3- to 12-membered heterocycloalkyl, or 5- to 10-membered heteroaryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}.

### Embodiment I-14

The compound of any one of Embodiments I-1 to I-12, wherein R⁴ is C₃₋₈cycloalkyl or C₆₋₁₀aryl, wherein C₃₋₈cycloalkyl and C₆₋₁₀aryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

### Embodiment I-15

The compound of any one of Embodiments I-1 to I-12, wherein R⁴ is phenyl optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

### Embodiment I-16

The compound of any one of Embodiments I-1 to I-3 and I-8 to 1-15, wherein T¹ is aryl or heteroaryl.

### Embodiment I-17

The compound of any one of Embodiments I-1 to I-3 and I-8 to I-15, wherein T¹ is 5- or 6-membered monocyclic heteroaryl or 9- or 10-membered bicyclic heteroaryl.

### Embodiment I-18

The compound of any one of Embodiments I-1 to I-3 and I-8 to 1-15, wherein T¹ is pyridinyl, pyrazinyl, pyridazinyl, pyrimidinyl, indolyl, indolinyl, isoindolyl, isoindolinyl, indazolyl, pyrazolopyridinyl, pyrazolopyrimidinyl, oxazolopyrimidinyl, oxazolopyridinyl, imidazopyridinyl, benzimidazolyl, tetrahydrobenzimidazolyl, benzofuranyl, dihydrobenzofuranyl, isobenzofuranyl, dihydroisobenzofuranyl, triazolopyridinyl, benzothiazolyl, azabenzimidazolyl, azabenzoxazolyl, azabenzothiazolyl, imidazopyridinyl, quinolinyl, isoquinolinyl, quinazolinyl, oxazolyl, isoxazolyl, thiazolyl, isothiazolyl, benzoxazolyl, benzodioxolyl, chromanyl, tetrahydrooxazoloazepinyl, tetrahydrobenzoxazolyl, oxadiazolyl, thiadiazolyl, pyrazolyl, triazolyl, imidazolyl, furanyl, or thiophenyl.

### Embodiment I-19

The compound of any one of Embodiments I-1 to I-3 and I-8 to I-15, wherein T¹ is 3- to 12-membered heterocycloalkyl.

### Embodiment I-20

The compound of Embodiment I-19, wherein T¹ is piperazine, piperidine, quinuclidine, or morpholine.

### Embodiment I-21

The compound of Embodiment I-1, wherein the compound is of Formula (IIa);

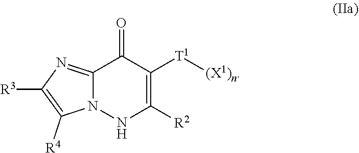

### Embodiment I-22

A compound of Formula (III):

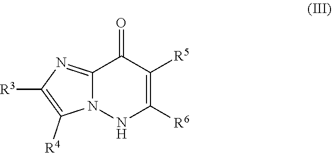

or a pharmaceutically acceptable salt, pharmaceutically acceptable solvate or pharmaceutically acceptable hydrate thereof, wherein

R⁵ is selected from the group consisting of —C(═O)NR⁹R¹⁰, phenyl optionally substituted with 1, 2, or 3 R¹¹, and a 5 to 10-membered heteroaryl optionally substituted with 1, 2, or 3 R¹²;

R⁶ is selected from the group consisting of —H, halo, —CN, C₁₋₃haloalkyl, and —C₁₋₃alkyl;

R⁷ is selected from the group consisting of —CN, —C₁₋₃alkyl, and C₁₋₃haloalkyl;

R⁸ is selected from the group consisting of phenyl optionally substituted with 1, 2, or 3 R¹³, and a 5- or 6-membered monocyclic heteroaryl optionally substituted with 1, 2, or 3 R¹⁴;

R⁹ and R¹⁰ are independently selected from the group consisting of —H, phenyl optionally substituted with 1, 2, or 3 R¹⁵, and a 5 to 10-membered heteroaryl optionally substituted with 1, 2, or 3 R¹⁶;

each R¹¹, R¹³ and R¹⁵ is independently selected from the group consisting of —C₁₋₃alkyl, —C₁₋₃haloalkyl, halo, —CN, —OH, —OC₁₋₃alkyl, and —OC₁₋₃haloalkyl; and

each R¹², R¹⁴ and R¹⁶ is independently selected from the group consisting of —C₁₋₃alkyl, —C₁₋₃haloalkyl, halo, —CN, —OH, —OC₁₋₃alkyl, and —OC₁₋₃haloalkyl.

### Embodiment I-23

The compound of Embodiment I-22, wherein R⁵ is selected from the group consisting of —C(═O)NR⁹R¹⁰, phenyl optionally substituted with 1, 2, or 3 R¹¹, a 5- or 6-membered monocyclic heteroaryl optionally substituted with 1, 2, or 3 R¹², and a 9- or 10-membered bicyclic heteroaryl optionally substituted with 1, 2, or 3 R¹²; and one of R⁹ and R¹⁰ is —H and the other of R⁹ and R¹⁰ is selected from the group consisting of phenyl optionally substituted with 1, 2, or 3 R¹⁵, a 5- or 6-membered monocyclic heteroaryl optionally substituted with 1, 2, or 3 R¹⁶, and a 9- or 10-membered bicyclic heteroaryl optionally substituted with 1, 2, or 3 R¹⁶.

### Embodiment I-24

The compound of Embodiment I-22, wherein R⁵ is selected from the group consisting of —C(═O)NR⁹R¹⁰, phenyl optionally substituted with 1, 2, or 3 R¹¹, a 5- or 6-membered monocyclic heteroaryl optionally substituted with 1, 2, or 3 R¹², and a 9- or 10-membered bicyclic heteroaryl optionally substituted with 1, 2, or 3 R¹²; one of R⁹ and R¹⁰ is —H and the other of R⁹ and R¹⁰ is selected from the group consisting of phenyl optionally substituted with 1, 2, or 3 R¹⁵, a 5- or 6-membered monocyclic heteroaryl optionally substituted with 1, 2, or 3 R¹⁶, and a 9- or 10-membered bicyclic heteroaryl optionally substituted with 1, 2, or 3 R¹⁶; R⁶ is C₁₋₃haloalkyl or —C₁₋₃alkyl, preferably —CH₃; R⁷ is —CN or —CF₃; and R^{e} is phenyl optionally substituted with 1, 2, or 3 substituents independently selected from the group consisting of —C₁₋₃alkyl, —C₁₋₃haloalkyl, halo, —CN, —OC₁₋₃alkyl, and —OC₁₋₃haloalkyl, preferably where R⁸ is unsubstituted phenyl.

### Embodiment I-25

The compound of Embodiment I-1, or a pharmaceutically acceptable salt, pharmaceutically acceptable solvate or pharmaceutically acceptable hydrate thereof, wherein the compound is selected from the group consisting of
N-(2,2-difluorobenzo[d][1,3]dioxol-4-yl)-6-methyl-8-oxo-3-phenyl-2-(trifluoromethyl)-5,8-dihydroimidazo[1,2-b]pyridazine-7-carboxamide (23);
N-(2-cyanophenyl)-6-methyl-8-oxo-3-phenyl-2-(trifluoromethyl)-5,8-dihydroimidazo[1,2-b]pyridazine-7-carboxamide (25);
N-(5-chloro-2-cyanophenyl)-6-methyl-8-oxo-3-phenyl-2-(trifluoromethyl)-5,8-dihydroimidazo[1,2-b]pyridazine-7-carboxamide (26);
N-(3-cyanopyridin-4-yl)-6-methyl-8-oxo-3-phenyl-2-(trifluoromethyl)-5,8-dihydroimidazo[1,2-b]pyridazine-7-carboxamide (27);
N-(2-cyano-5-fluorophenyl)-6-methyl-8-oxo-3-phenyl-2-(trifluoromethyl)-5,8-dihydroimidazo[1,2-b]pyridazine-7-carboxamide (28);
N-(2-cyano-5-fluoropyridin-3-yl)-6-methyl-8-oxo-3-phenyl-2-(trifluoromethyl)-5,8-dihydroimidazo[1,2-b]pyridazine-7-carboxamide (29);
N-(2-cyano-4-fluorophenyl)-6-methyl-8-oxo-3-phenyl-2-(trifluoromethyl)-5,8-dihydroimidazo[1,2-b]pyridazine-7-carboxamide (30);
N-(2-cyano-3-fluorophenyl)-6-methyl-8-oxo-3-phenyl-2-(trifluoromethyl)-5,8-dihydroimidazo[1,2-b]pyridazine-7-carboxamide (31);
N-(4-cyanopyridin-3-yl)-6-methyl-8-oxo-3-phenyl-2-(trifluoromethyl)-5,8-dihydroimidazo[1,2-b]pyridazine-7-carboxamide (32);
6-methyl-8-oxo-3-phenyl-2-(trifluoromethyl)-N-(4-(trifluoromethyl)thiazol-2-yl)-5,8-dihydroimidazo[1,2-b]pyridazine-7-carboxamide (33);
2-cyano-N-(2-cyano-5-fluoropyridin-3-yl)-6-methyl-8-oxo-3-phenyl-5,8-dihydroimidazo[1,2-b]pyridazine-7-carboxamide (34);
2-cyano 1N-(2-cyanopyridin-3-yl)-6-methyl-8-oxo-3-phenyl-5,8-dihydroimidazo[1,2-b]pyridazine-7-carboxamide (35);
2-cyano-N-(3-cyanopyridin-4-yl)-6-methyl-8-oxo-3-phenyl-5,8-dihydroimidazo[1,2-b]pyridazine-7-carboxamide (36);
2-cyano-6-methyl-8-oxo-3-phenyl-N-(2-(trifluoromethyl)thiazol-4-yl)-5,8-dihydroimidazo[1,2-b]pyridazine-7-carboxamide (37);
N-(5-chloro-2-cyanophenyl)-2-cyano-6-methyl-8-oxo-3-phenyl-5,8-dihydroimidazo[1,2-b]pyridazine-7-carboxamide (38);
2-cyano-N-(2-cyano-5-fluorophenyl)-6-methyl-8-oxo-3-phenyl-5,8-dihydroimidazo[1,2-b]pyridazine-7-carboxamide (39);
2-cyano-N-(2-cyano-3-fluorophenyl)-6-methyl-8-oxo-3-phenyl-5,8-dihydroimidazo[1,2-b]pyridazine-7-carboxamide (40);
2-cyano-N-(2-cyano-4-fluorophenyl)-6-methyl-8-oxo-3-phenyl-5,8-dihydroimidazo[1,2-b]pyridazine-7-carboxamide (41);
2-cyano-6-methyl-8-oxo-3-phenyl-N-(6-(trifluoromethyl)pyridin-2-yl)-5,8-dihydroimidazo[1,2-b]pyridazine-7-carboxamide (42);
2-cyano-N-(3-cyanothiophen-2-yl)-6-methyl-8-oxo-3-phenyl-5,8-dihydroimidazo[1,2-b]pyridazine-7-carboxamide (43);
N-(4-chloro-2-cyanophenyl)-2-cyano-6-methyl-8-oxo-3-phenyl-5,8-dihydroimidazo[1,2-b]pyridazine-7-carboxamide (44); and
2-cyano-N-(3-cyanopyridazin-4-yl)-6-methyl-8-oxo-3-phenyl-5,8-dihydroimidazo[1,2-b]pyridazine-7-carboxamide (45); represented by the structures

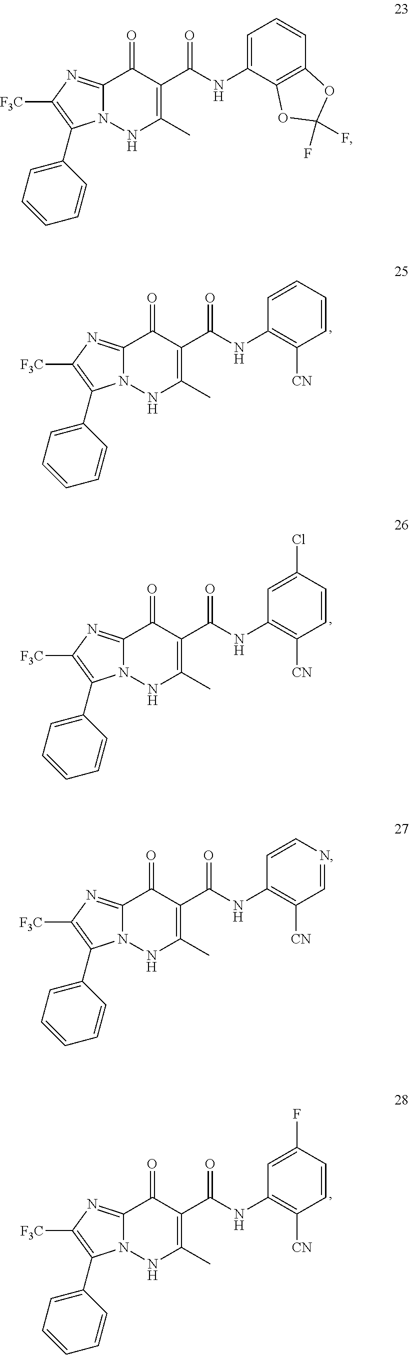 [SMILES: Cc1[nH]n2c(-c3ccccc3)c(C(F)(F)F)nc2c(=O)c1C(=O)Nc1cccc2c1OC(F)(F)O2 ; Cc1[nH]n2c(-c3ccccc3)c(C(F)(F)F)nc2c(=O)c1C(=O)Nc1ccccc1C#N ; Cc1[nH]n2c(-c3ccccc3)c(C(F)(F)F)nc2c(=O)c1C(=O)Nc1cc(Cl)ccc1C#N ; Cc1[nH]n2c(-c3ccccc3)c(C(F)(F)F)nc2c(=O)c1C(=O)Nc1ccncc1C#N ; Cc1[nH]n2c(-c3ccccc3)c(C(F)(F)F)nc2c(=O)c1C(=O)Nc1cc(F)ccc1C#N]

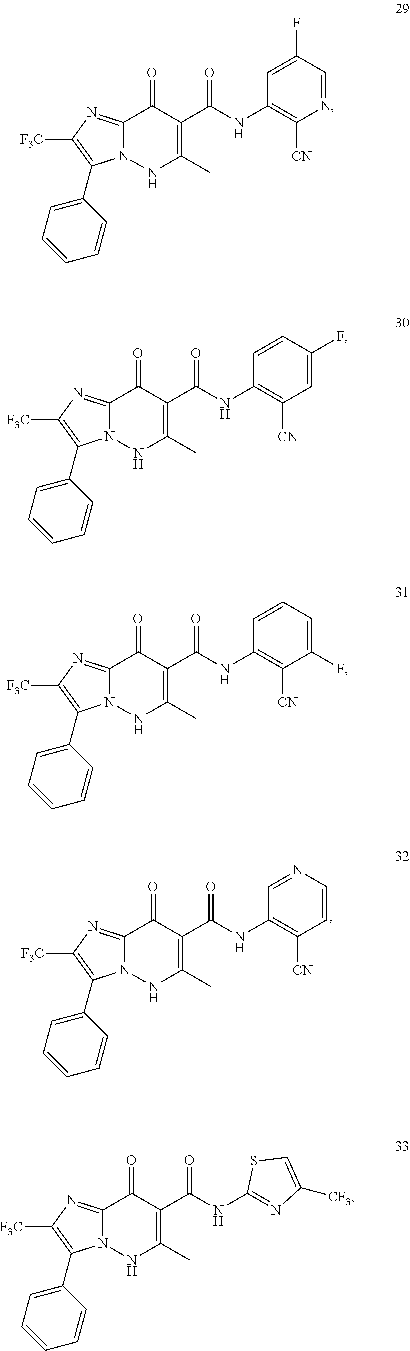 [SMILES: Cc1[nH]n2c(-c3ccccc3)c(C(F)(F)F)nc2c(=O)c1C(=O)Nc1cc(F)cnc1C#N ; Cc1[nH]n2c(-c3ccccc3)c(C(F)(F)F)nc2c(=O)c1C(=O)Nc1ccc(F)cc1C#N ; Cc1[nH]n2c(-c3ccccc3)c(C(F)(F)F)nc2c(=O)c1C(=O)Nc1cccc(F)c1C#N ; Cc1[nH]n2c(-c3ccccc3)c(C(F)(F)F)nc2c(=O)c1C(=O)Nc1cnccc1C#N ; Cc1[nH]n2c(-c3ccccc3)c(C(F)(F)F)nc2c(=O)c1C(=O)Nc1nc(C(F)(F)F)cs1]

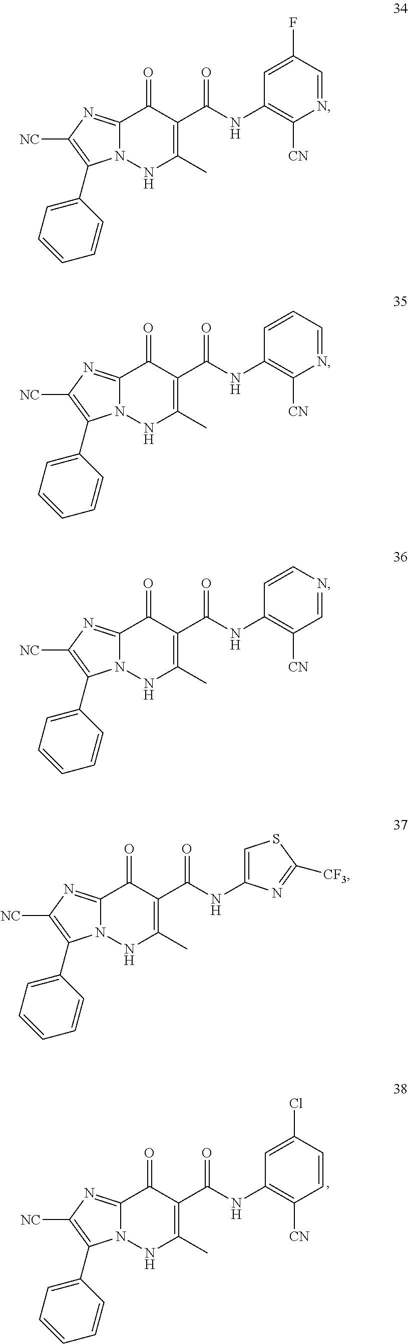 [SMILES: Cc1[nH]n2c(-c3ccccc3)c(C#N)nc2c(=O)c1C(=O)Nc1cc(F)cnc1C#N ; Cc1[nH]n2c(-c3ccccc3)c(C#N)nc2c(=O)c1C(=O)Nc1cccnc1C#N ; Cc1[nH]n2c(-c3ccccc3)c(C#N)nc2c(=O)c1C(=O)Nc1ccncc1C#N ; Cc1[nH]n2c(-c3ccccc3)c(C#N)nc2c(=O)c1C(=O)Nc1csc(C(F)(F)F)n1 ; Cc1[nH]n2c(-c3ccccc3)c(C#N)nc2c(=O)c1C(=O)Nc1cc(Cl)ccc1C#N]

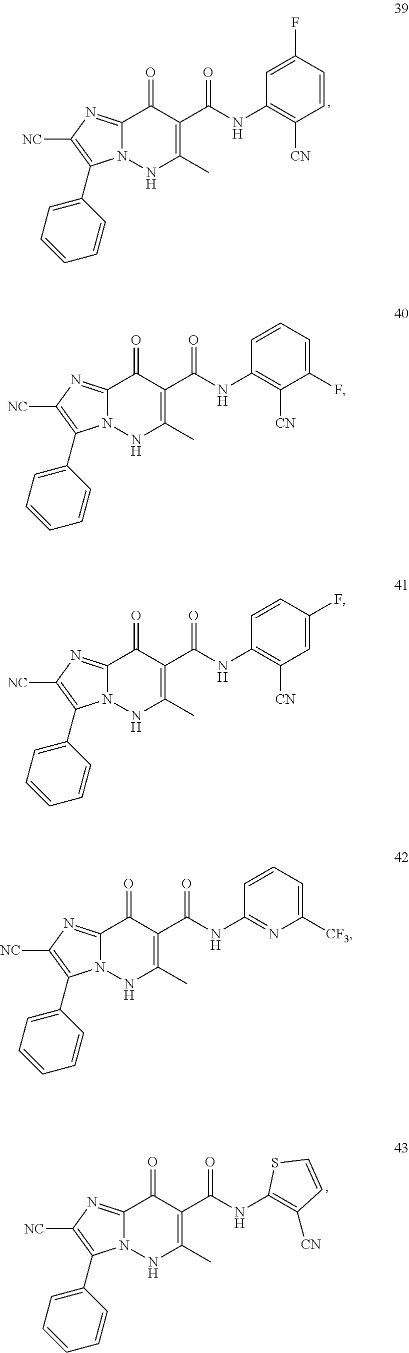 [SMILES: Cc1[nH]n2c(-c3ccccc3)c(C#N)nc2c(=O)c1C(=O)Nc1cc(F)ccc1C#N ; Cc1[nH]n2c(-c3ccccc3)c(C#N)nc2c(=O)c1C(=O)Nc1cccc(F)c1C#N ; Cc1[nH]n2c(-c3ccccc3)c(C#N)nc2c(=O)c1C(=O)Nc1ccc(F)cc1C#N ; Cc1[nH]n2c(-c3ccccc3)c(C#N)nc2c(=O)c1C(=O)Nc1cccc(C(F)(F)F)n1 ; Cc1[nH]n2c(-c3ccccc3)c(C#N)nc2c(=O)c1C(=O)Nc1sccc1C#N]

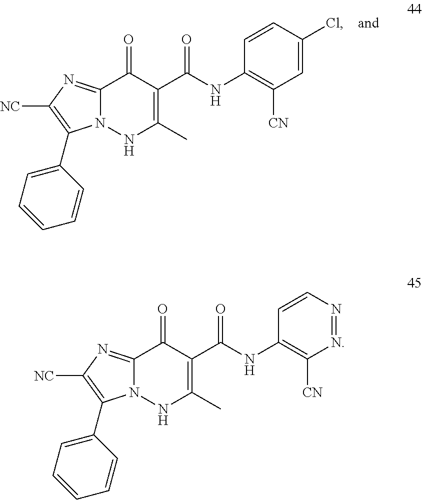 [SMILES: Cc1[nH]n2c(-c3ccccc3)c(C#N)nc2c(=O)c1C(=O)Nc1ccc(Cl)cc1C#N ; "and" ; Cc1[nH]n2c(-c3ccccc3)c(C#N)nc2c(=O)c1C(=O)Nc1ccnnc1C#N]

### Embodiment I-25a

The compound of Embodiment I-1, or a pharmaceutically acceptable salt, pharmaceutically acceptable solvate or pharmaceutically acceptable hydrate thereof, wherein the compound is selected from the group consisting of
7-(3-methoxyphenyl)-6-methyl-8-oxo-3-phenyl-5,8-dihydroimidazo[1,2-b]pyridazine-2-carbonitrile (5);
7-(3-chlorophenyl)-6-methyl-8-oxo-3-phenyl-5,8-dihydroimidazo[1,2-b]pyridazine-2-carbonitrile (6);
7-(4-ethylphenyl)-6-methyl-8-oxo-3-phenyl-5,8-dihydroimidazo[1,2-b]pyridazine-2-carbonitrile (13);
7-(2-fluorophenyl)-6-methyl-8-oxo-3-phenyl-5,8-dihydroimidazo[1,2-b]pyridazine-2-carbonitrile (17);
7-(3,5-difluorophenyl)-6-methyl-3-phenyl-2-(trifluoromethyl)imidazo[1,2-b]pyridazin-8(5H)-one (18);
7-(3-methoxyphenyl)-6-methyl-3-phenyl-2-(trifluoromethyl)imidazo[1,2-b]pyridazin-8(5H)-one (19);
7-(3-bromophenyl)-6-methyl-3-phenyl-2-(trifluoromethyl)imidazo[1,2-b]pyridazin-8(5H)-one (20);
7-(4-ethylphenyl)-6-methyl-3-phenyl-2-(trifluoromethyl)imidazo[1,2-b]pyridazin-8(5H)-one (21); and
7-(1H-indol-5-yl)-6-methyl-3-phenyl-2-(trifluoromethyl)imidazo[1,2-b]pyridazin-8(5H)-one (22); represented by the structures

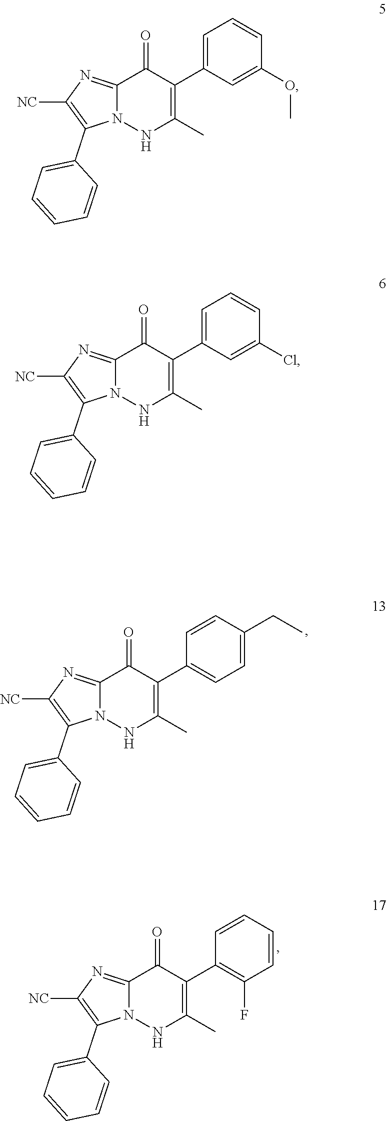 [SMILES: COc1cccc(-c2c(C)[nH]n3c(-c4ccccc4)c(C#N)nc3c2=O)c1 ; Cc1[nH]n2c(-c3ccccc3)c(C#N)nc2c(=O)c1-c1cccc(Cl)c1 ; CCc1ccc(-c2c(C)[nH]n3c(-c4ccccc4)c(C#N)nc3c2=O)cc1 ; Cc1[nH]n2c(-c3ccccc3)c(C#N)nc2c(=O)c1-c1ccccc1F]

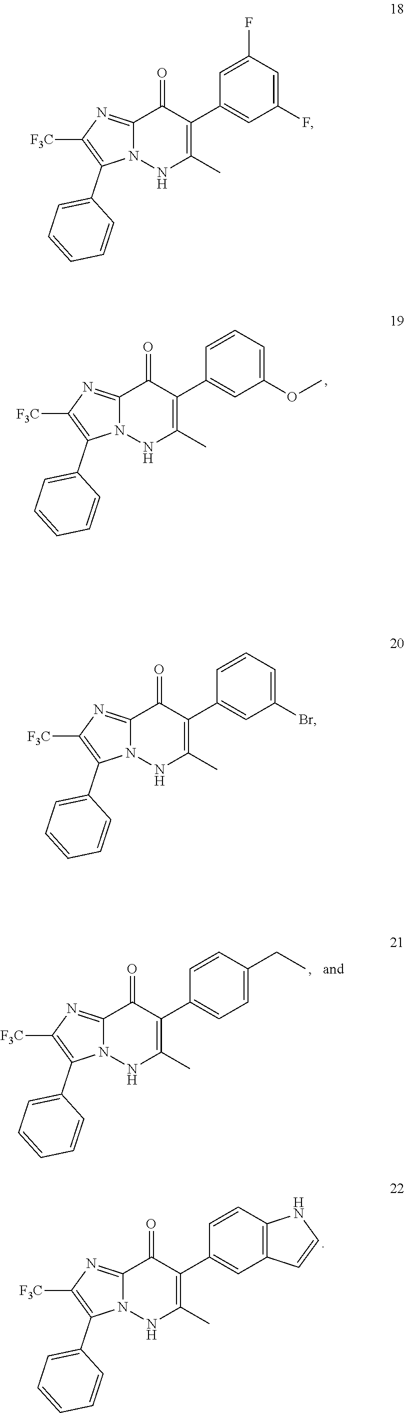 [SMILES: Cc1[nH]n2c(-c3ccccc3)c(C(F)(F)F)nc2c(=O)c1-c1cc(F)cc(F)c1 ; COc1cccc(-c2c(C)[nH]n3c(-c4ccccc4)c(C(F)(F)F)nc3c2=O)c1 ; Cc1[nH]n2c(-c3ccccc3)c(C(F)(F)F)nc2c(=O)c1-c1cccc(Br)c1 ; CCc1ccc(-c2c(C)[nH]n3c(-c4ccccc4)c(C(F)(F)F)nc3c2=O)cc1 ; Cc1[nH]n2c(-c3ccccc3)c(C(F)(F)F)nc2c(=O)c1-c1ccc2[nH]ccc2c1]

### Embodiment I-26

A pharmaceutical composition comprising the compound of any one of the preceding embodiments, or a pharmaceutically acceptable salt, pharmaceutically acceptable solvate or pharmaceutically acceptable hydrate thereof, together with a pharmaceutically acceptable diluent or carrier.

### Embodiment I-27

A method of inhibiting the cGAS/STING pathway in a cell, comprising contacting the cell with the compound of any one of Embodiments I-1 to I-25, or a pharmaceutically acceptable salt, pharmaceutically acceptable solvate or pharmaceutically acceptable hydrate thereof, or the composition of Embodiment I-26.

### Embodiment I-28

A method of inhibiting cytokine production in a cell, comprising contacting the cell with the compound of any one of Embodiments I-1 to I-25, or pharmaceutically acceptable salt, pharmaceutically acceptable solvate or pharmaceutically acceptable hydrate thereof, or the composition of Embodiment I-26.

### Embodiment I-29

A method of treating a cGAS/STING pathway-mediated condition, comprising administering to a subject in need thereof an effective amount of a compound of any one of Embodiments I-1 to I-25, or a pharmaceutically acceptable salt, pharmaceutically acceptable solvate or pharmaceutically acceptable hydrate thereof, or the composition of Embodiment I-26.

### Embodiment I-30

The method of Embodiment I-29, wherein the cGAS/STING pathway-mediated condition is an autoimmune, inflammatory, or neurodegenerative condition.

### Embodiment I-31

A method of treating a disease in a subject, comprising administering to the subject in need thereof a therapeutically effective amount of the compound of any one of Embodiments I-1 to I-25, or a pharmaceutically acceptable salt, pharmaceutically acceptable solvate or pharmaceutically acceptable hydrate thereof, or the composition of Embodiment I-26, wherein the disease is selected from the group consisting of systemic inflammatory response syndrome (SIRS), sepsis, septic shock, atherosclerosis, celiac disease, dermatomyositis, scleroderma, interstitial cystitis, transplant rejection (e.g. graft-versus-host disease), Aicardi-Goutieres Syndrome, Hutchison Guilford progeria syndrome, Singleton-Merten Syndrome, proteasome-associated autoinflammatory syndrome, SAVI (STING-associated vasculopathy with onset in infancy), CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis. Crohn's disease), idiopathic thrombocytopenic purpura, thrombotic thrombocytopenic purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes. Type 2 diabetes, Sjogren's syndrome, X-linked reticulate pigmentary disorder, polymyositis, spondyloenchondrodysplasia, age-related macular degeneration, Alzheimer's disease and Parkinson's disease.

### Embodiment I-32

A method of treating a disease in a subject, comprising administering to the subject in need thereof a therapeutically effective amount of the compound of any one of Embodiments I-1 to I-25, including any pharmaceutically acceptable salt, pharmaceutically acceptable solvate or pharmaceutically acceptable hydrate thereof, or the composition of Embodiment I-26, in combination with a Janus Kinase (Jak) inhibitor, wherein the disease is selected from the group consisting of systemic inflammatory response syndrome (SIRS), sepsis, septic shock, atherosclerosis, celiac disease, dermatomyositis, scleroderma, interstitial cystitis, transplant rejection (e.g. graft-versus-host disease), Aicardi-Goutieres Syndrome, Hutchison Guilford progeria syndrome, Singleton-Merten Syndrome, proteasome-associated autoinflammatory syndrome, SAVI (STING-associated vasculopathy with onset in infancy), CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenic purpura, thrombotic thrombocytopenic purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjogren's syndrome, X-linked reticulate pigmentary disorder, polymyositis, spondyloenchondrodysplasia, age-related macular degeneration. Alzheimer's disease and Parkinson's disease.

### Embodiment I-33

A compound of any one of Embodiments I-1 to I-25, for use in the treatment of a cGAS/STING pathway-mediated condition.

### Embodiment I-34

A compound of any one of Embodiments I-1 to I-25, for use in combination with a Janus Kinase inhibitor, for the treatment of a disease selected from the group consisting of systemic inflammatory response syndrome (SIRS), sepsis, septic shock, atherosclerosis, celiac disease, dermatomyositis, scleroderma, interstitial cystitis, transplant rejection (e.g. graft-versus-host disease), Aicardi-Goutieres Syndrome. Hutchison Guilford progeria syndrome, Singleton-Merten Syndrome, proteasome-associated autoinflammatory syndrome, SAVI (STING-associated vasculopathy with onset in infancy), CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenic purpura, thrombotic thrombocytopenic purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjogren's syndrome. X-linked reticulate pigmentary disorder, polymyositis, spondyloenchondrodysplasia, age-related macular degeneration. Alzheimer's disease and Parkinson's disease.

### Embodiment I-35

Composition comprising a compound of any one of Embodiments I-1 to i-25 and a Janus Kinase inhibitor.

### Embodiment I-36

A kit comprising a compound of any one of Embodiments I-1 to I-25 and a Janus Kinase inhibitor.

### EXAMPLES

NMR spectra were recorded on a Bruker Avance II Ultra shield spectrometer (500 MHz), or Bruker Avance III HD (400 mHz). LCMS were acquired on a Waters Alliance 2695 with column heater LC system equipped with a Waters PDA 996 (210-300 nm) UV detector and a Waters ZQ 2000, ESI (ES+, 100-1200 amu) MS Detector. Mobile phases (Mobile phase A: Milli-Q H2O+10 mM Ammonium Formate pH: 3.8 (Am.F.), or Ammonium Bicarbonate pH: 10 (Am.B.), Mobile Phase B: CH₃CN). LC conditions are: XBridge C₁₈, 3.5 μm, 4.6×30 mm; Iso 5% B for 0.5 min, 5% to 100% B in 5 minutes; hold 100% B for 2 minutes; flow rate: 3 mL/min. The methods described in the examples below can be readily modified by one skilled in the art. Compounds made similarly to the exemplified methods may include modifications of reaction conditions, such as any one or more of the reagent concentrations, solvents, reaction times, temperatures, work-up conditions, purification conditions, and the like to provide additional compounds of the invention as described herein.

Abbreviations and Acronyms. AcOH=acetic acid, Burgess reagent=1-Methoxy-N-triethylammoniosulfonyl-methanimidate, DCM=CH₂Cl₂=dichloromethane, DBU=1,8-diazabicyclo[5.4.0]undec-7-ene, DIPEA=N,N-Diisopropylethylamine, DMEDA=N,N′-Dimethylethylenediamine, DMF=Dimethylformamide, DMSO=Dimethyl sulfoxide, EtOH=ethanol, EtOAc=ethyl acetate, FBS=Fetal Bovine Serum, HATU (Hexafluorophosphate Azabenzotriazole Tetramethyl Uronium)=1-[Bis(dimethylamino)ₘethylene]-1H-1,2,3-triazolo[4,5-b]pyridinium 3-oxide hexafluorophosphate, LAH=Lithium aluminum hydride, LTB=Lithium tert-butoxide, MeCN=acetonitrile, MeOH=methanol, NaOMe=Sodium methoxide, NBS=N-Bromosuccinimide, NIS=N-Iodosuccinimide, Pyr-SO₃=sulfur trioxide pyridine complex, Pd(dppf)Cl₂=[1,1′-bis(diphenylphosphino)ferrocene]dichloropalladium(II), TEA=triethylamine, TFAA=trifluoroacetic anhydride. TFA=trifluoroacetic acid, THF=tetrahydrofuran, TMB=trimethylboroxine, TEMPO=(2,2,6,6-Tetramethylpiperidin-1-yl)oxyl, Fe(acac)₃=Iron(III) acetylacetonate.

### Example 1

Synthesis of compounds 1-6 was carried out in two, three, four, or five steps as follows:

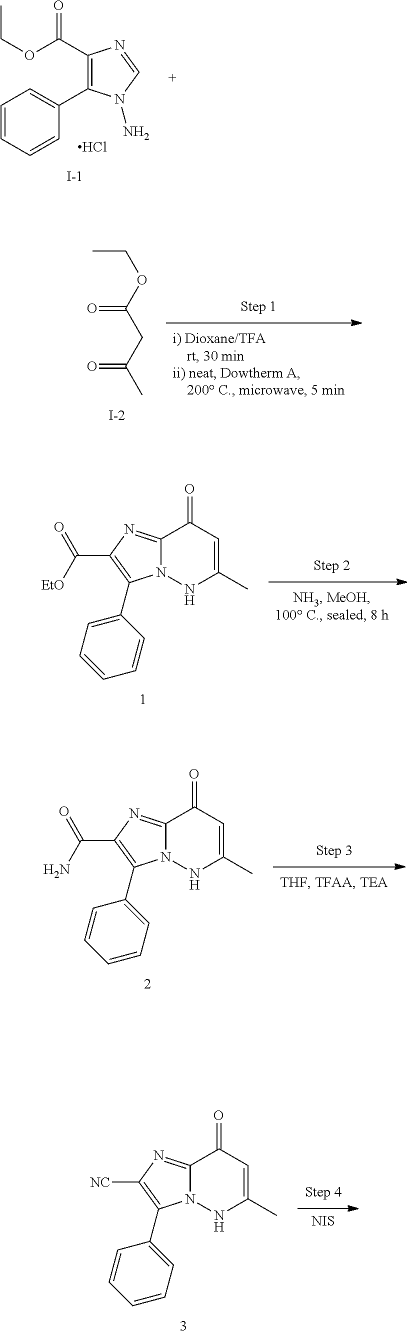

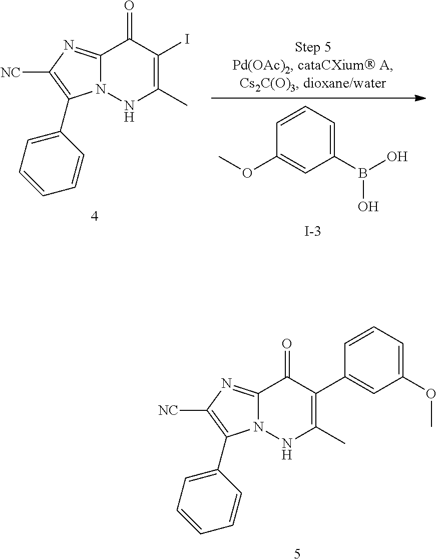

### Step 1

Synthesis of ethyl 6-methyl-8-oxo-3-phenyl-5,8-dihydroimidazo[1,2-b]pyridazine-2-carboxylate (1): Ethyl 1-amino-5-phenyl-1H-imidazole-4-carboxylate hydrochloride (1-1, 300 mg, 1.12 mmol, 1.0 equiv., synthesized according to Yamada. M.; Fukui, T.; Nunami, K. Synthesis 1995, 1365) was dissolved in dioxane (3.7 mL) in a 50 mL round bottom flask. Ethyl acetoacetate (1-2, 213 μL, 1.68 mmol, 1.50 equiv.) was added, followed by trifluoroacetic acid (0.8 mL). The flask was capped and the mixture was stirred at room temperature for 30 min. The solvents were removed in vacuo. The residue was azeotroped twice with toluene. The resulting oil was dissolved in Dowtherm A® (4 mL) and heated in a microwave reactor at 200° C., for 5 min. After cooling to room temperature, the reaction mixture was added to 40 mL hexanes and 80 mL MeCN. The layers were separated and the MeCN layer was washed three times with 80 mL hexanes. The MeCN layer was concentrated in vacuo and the crude mixture purified by reverse phase flash chromatography (C18 column) using a gradient of MeCN (+0.1% formic acid) in water (+0.1% formic acid). The fractions containing the desired compound were partially concentrated, then lyophilized, to give compound 1 as a tan solid (108 mg, 0.36 mmol, 32% yield). LC-MS (ESI): 98% purity, m/z=298.1 [M+H]. ¹H NMR (400 MHz, DMSO): δ 7.60-7.48 (m, 5H), 6.45 (s, 1H), 4.19 (q, J=7.1 Hz, 2H), 2.34 (s, 3H), 1.16 (t, J=7.1 Hz, 3H).

### Step 2

Synthesis of 6-methyl-8-oxo-3-phenyl-5,8-dihydroimidazo[1,2-b]pyridazine-2-carboxamide (2): Compound 1 (108 mg, 0.36 mmol) was dissolved in 7N NH₃ in MeOH (4.0 mL) in a 15 mL heavy wall glass tube equipped with a Teflon cap and o-ring. The tube was sealed and heated to 100° C., for 24 h. After cooling to room temperature, the solvent was removed in vacuo and the material was purified by reverse phase flash chromatography (C18 column) using a gradient of MeCN (+0.1% formic acid) in water (+0.1% formic acid). The fractions containing the desired compound were partially concentrated, then lyophilized, to give compound 2 as a yellow solid (74.0 mg, 0.27 mmol, 76% yield). LC-MS (ESI): 96% purity, m/z=269.0 [M+H].

### Step 3

Synthesis of 6-methyl-8-oxo-3-phenyl-5,8-dihydroimidazo[1,2-b]pyridazine-2-carbonitrile (3): Compound 2 (71 mg, 0.27 mmol, 1.0 equiv.) was suspended in THF (5.0 mL). Triethylamine (186 μL, 1.32 mmol, 5.0 equiv.) was added and the mixture was cooled to 0° C. Trifluoroacetic anhydride (74 μL, 0.53 mmol, 2.0 equiv.) was added and the mixture was warmed to room temperature. After 30 min and recooling to 0° C., an additional 5.0 equiv. of TEA and 2.0 equiv. of TFAA were added. After 30 min, the reaction was concentrated in vacuo to an oil and loaded directly (with DMSO) on a C18 reverse phase column for purification using a gradient of MeCN (+0.1% formic acid) in water (+0.1% formic acid). The fractions containing the desired compound were partially concentrated, then lyophilized, to give the compound 3 as a yellow solid (30.0 mg, 0.12 mmol, 45% yield). LC-MS: 99% purity, m/z=251.2 [M+H]. ¹H NMR (500 MHz, DMSO): δ 7.96-7.90 (m, 2H), 7.65-7.60 (m, 2H), 7.58-7.52 (m, 1H), 6.51 (s, 1H), 2.42 (s, 3H).

### Step 4

Synthesis of 7-iodo-6-methyl-8-oxo-3-phenyl-5,8-dihydroimidazo[1,2-b]pyridazine-2-carbonitrile (4): Compound 3 (29.0 mg, 0.12 mmol, 1.0 equiv.) was dissolved in DMF (580 μL) and cooled to 0° C. N-iodosuccinimide (26.9 mg, 0.12 mmol, 1.0 equiv.) was added and the mixture was stirred at 0° C., for 10 min. Water (2 mL) was added, and the resulting precipitate was filtered and air-dried to afford the desired compound 4 as an off-white solid (40.0 mg, 0.12 mmol, 92% yield) LC-MS: 98% purity, m/z=376.9 [M+H]. ¹H NMR (500 MHz, DMSO): δ 7.96-7.93 (m, 2H), 7.66-7.62 (m, 2H), 7.59-7.55 (m, 1H), 2.66 (s, 3H).

### Step 5

Synthesis of 7-(3-methoxyphenyl)-6-methyl-8-oxo-3-phenyl-5,8-dihydroimidazo[1,2-b]pyridazine-2-carbonitrile (5): Compound 4 (26.0 mg, 0.069 mmol, 1.0 equiv.), 3-methoxyphenyl boronic acid (I-3, 15.7 mg, 0.10 mmol, 1.5 equiv.), palladium acetate (1.6 mg, 0.0069 mol, 10 mol %), cataCXium® A (5.2 mg, 0.014 mmol, 20 mol %) and cesium carbonate (57 mg, 0.17 mmol, 2.5 equiv.) were added to a screw cap tube. After three vacuum/N₂ cycles, degassed dioxane (460 μL) and water (230 μL) were added. The tube was capped under N₂ and heated to 100° C., for 2 h. The reaction was diluted with EtOAc and poured on saturated NH₄Cl (aq). The layers were separated and the aqueous layer was extracted three times with EtOAc. The combined organic layers were washed with brine, dried over MgSO₄, filtered, and concentrated in vacuo. The crude material was purified by reverse phase HPLC (C18 column) using a gradient of MeCN in 10 mM ammonium bicarbonate (aq). The fractions containing the desired compound were partially concentrated, then lyophilized, to give compound 5 as a white solid (6.0 mg, 0.017 mmol, 24% yield). LC-MS: 100% purity, m/z=357.1 [M+H]. ¹H NMR (500 MHz, DMSO): δ 8.02 (d, J=7.3 Hz, 2H), 7.56 (t, J=7.7 Hz, 2H), 7.45 (t, J=7.4 Hz, 1H), 7.24 (t, J=7.9 Hz, 1H), 6.86-6.66 (m, 3H), 3.75 (s, 3H), 2.05 (s, 3H).

The compound 7-(3-chlorophenyl)-6-methyl-8-oxo-3-phenyl-5,8-dihydroimidazo[1,2-b]pyridazine-2-carbonitrile (6)

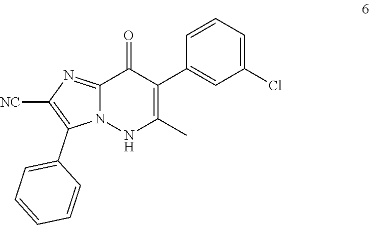 [SMILES: Cc1[nH]n2c(-c3ccccc3)c(C#N)nc2c(=O)c1-c1cccc(Cl)c1]

was prepared similarly, replacing 3-methoxyphenyl boronic acid 1-3 with 3-chlorophenyl boronic acid. Compound 4 (40.0 mg, 0.11 mmol, 1.0 equiv.), 3-chlorophenyl boronic acid (24.9 mg, 0.16 mmol, 1.5 equiv.), palladium acetate (2.4 mg, 0.011 mol, 10 mol %); cataCXium® A (8.0 mg, 0.021 mmol, 20 mol %) and cesium carbonate (88 mg, 0.27 mmol, 2.5 equiv.) were added to a screw cap tube. After three vacuum/N₂ cycles, degassed dioxane (709 μL) and water (354 μL) were added. The tube was capped under N₂ and heated to 100° C., for 3 h. The reaction was diluted with EtOAc and poured on saturated NH₄Cl (aq). The layers were separated and the aqueous layer was extracted three times with EtOAc. The combined organic layers were washed with brine, dried over MgSO₄, filtered, and concentrated in vacuo. The crude material was purified by reverse phase flash chromatography (C18 column) using a gradient of MeCN (+0.1% formic acid) in water (+0.1% formic acid). The fractions containing the desired compound were partially concentrated, then lyophilized, to give compound 6 as a white solid (15 mg, 0.043 mmol, 39% yield). LC-MS: 100% purity, m/z=361.8 [M+H]. ¹H NMR (400 MHz, DMSO): δ 8.04-7.97 (m, 2H), 7.60-7.55 (m, 2H), 7.50-7.43 (m, 1H), 7.37 (t, J=7.8 Hz; 1H), 7.34-7.30 (m, 1H), 7.28-7.25 (m, 1H), 7.23-7.19 (m, 1H), 2.08 (s, 3H).

The compound 7-(4-ethylphenyl)-6-methyl-8-oxo-3-phenyl-5,8-dihydroimidazo[1,2-b]pyridazine-2-carbonitrile (13)

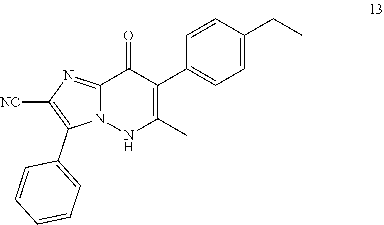 [SMILES: CCc1ccc(-c2c(C)[nH]n3c(-c4ccccc4)c(C#N)nc3c2=O)cc1]

was prepared similarly, replacing 3-methoxyphenyl boronic acid 1-3 with 4-ethylphenyl boronic acid. Compound 4 (30.0 mg, 0.080 mmol, 1.0 equiv.), 4-ethylphenyl boronic acid (17.9 mg, 0.12 mmol, 1.5 equiv.), palladium acetate (1.8 mg, 0.0080 mol, 10 mol %), cataCXium® A (6.0 mg, 0.016 mmol, 20 mol %) and cesium carbonate (66 mg, 0.20 mmol, 2.5 equiv.) were added to a screw cap tube. After three vacuum/N₂ cycles, degassed dioxane (530 μL) and water (265 μL) were added. The tube was capped under N₂ and heated to 100° C., for 1 h. The reaction was diluted with EtOAc and poured on saturated NH₄Cl (aq). The layers were separated and the aqueous layer was extracted three times with EtOAc. The combined organic layers were washed with brine, dried over MgSO₄, filtered, and concentrated in vacuo. The crude material was purified by reverse phase flash chromatography (C18 column) using a gradient of MeCN (+0.1% formic acid) in water (+0.1% formic acid). The fractions containing the desired compound were partially concentrated, then lyophilized, to give compound 13 as a white solid (8.0 mg, 0.022 mmol, 28% yield). LC-MS: 95.6% purity, m/z=355.2 [M+H]. ¹H NMR (400 MHz, DMSO): δ 8.02-7.98 (m, 2H), 7.64 (t, J=7.5 Hz, 2H), 7.60-7.54 (m, 1H), 7.32 (d, J=7.9 Hz, 2H), 7.26 (d, J=7.9 Hz, 2H), 2.68 (q, J=7.6 Hz, 2H), 2.21 (s, 3H), 1.25 (t, J=7.6 Hz, 3H).

The compound 7-(2-fluorophenyl)-6-methyl-8-oxo-3-phenyl-5,8-dihydroimidazo[1,2-b]pyridazine-2-carbonitrile (17)

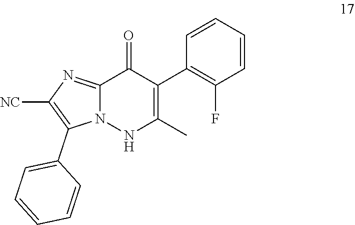 [SMILES: Cc1[nH]n2c(-c3ccccc3)c(C#N)nc2c(=O)c1-c1ccccc1F]

was prepared similarly, replacing 3-methoxyphenyl boronic acid I-3 with 2-fluorophenyl boronic acid. LC-MS: 99.6% purity, m/z=345.2 [M+H]. ¹H NMR (400 MHz, DMSO): δ 8.03-8.00 (m, 2H), 7.60-7.56 (m, 2H), 7.50-7.48 (m, 1H), 7.34-7.27 (m, 2H), 7.22-7.19 (, 2H), 2.02 (s, 3H).

### Example 2: Additional Compounds

The compounds 7-(3-chlorophenyl)-3-phenyl-6-(2H-tetrazol-5-yl)-2-(trifluoromethyl)imidazo[1,2-b]pyridazin-8(5H)-one (7), 7-(3-chlorophenyl)-8-oxo-3-phenyl-6-(2H-tetrazol-5-yl)-5,8-dihydroimidazo[1,2-b]pyridazine-2-carbonitrile (8), 7-(3-methoxyphenyl)-3-phenyl-6-(2H-tetrazol-5-yl)-2-(trifluoromethyl)imidazo[1,2-b]pyridazin-8(5H)-one (9), 7-(3-methoxyphenyl)-8-oxo-3-phenyl-6-(2H-tetrazol-5-yl)-5,8-dihydroimidazo[1,2-b]pyridazine-2-carbonitrile (10), 7-(4-methoxybenzo[d]oxazol-2-yl)-3-phenyl-6-(2H-tetrazol-5-yl)-2-(trifluoromethyl)imidazo[1,2-b]pyridazin-8(5H)-one (11), and 7-(4-methoxybenzo[d]oxazol-2-yl)-8-oxo-3-phenyl-6-(2H-tetrazol-5-yl)-5,8-dihydroimidazo[1,2-b]pyridazine-2-carbonitrile (12);

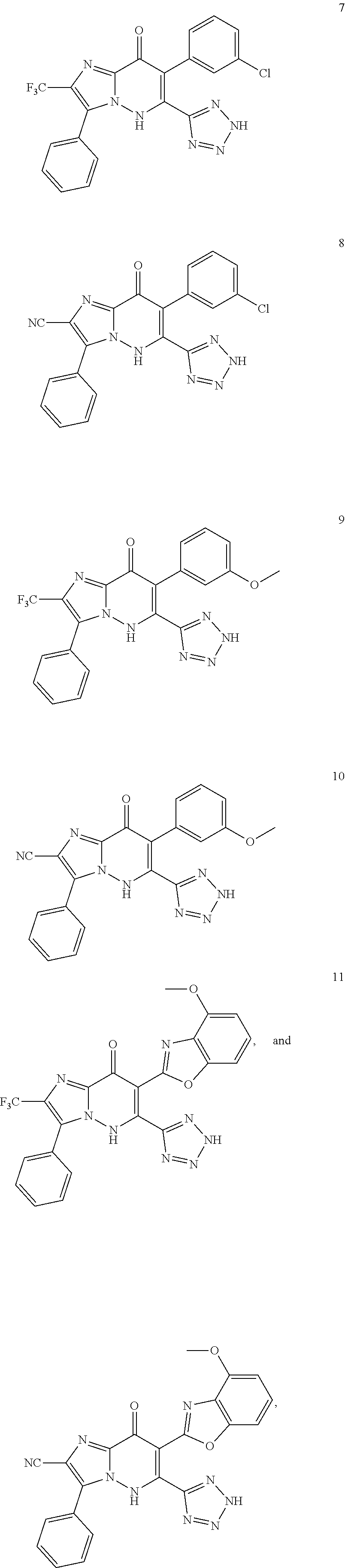 [SMILES: O=c1c(-c2cccc(Cl)c2)c(-c2nn[nH]n2)[nH]n2c(-c3ccccc3)c(C(F)(F)F)nc12 ; N#Cc1nc2c(=O)c(-c3cccc(Cl)c3)c(-c3nn[nH]n3)[nH]n2c1-c1ccccc1 ; COc1cccc(-c2c(-c3nn[nH]n3)[nH]n3c(-c4ccccc4)c(C(F)(F)F)nc3c2=O)c1 ; COc1cccc(-c2c(-c3nn[nH]n3)[nH]n3c(-c4ccccc4)c(C#N)nc3c2=O)c1 ; COc1cccc2oc(-c3c(-c4nn[nH]n4)[nH]n4c(-c5ccccc5)c(C(F)(F)F)nc4c3=O)nc12 ; COc1cccc2oc(-c3c(-c4nn[nH]n4)[nH]n4c(-c5ccccc5)c(C#N)nc4c3=O)nc12]

are prepared similarly to the methods as described herein.

### Example 3

Synthesis of compounds 14-16 was carried out in four, five, or six steps as follows:

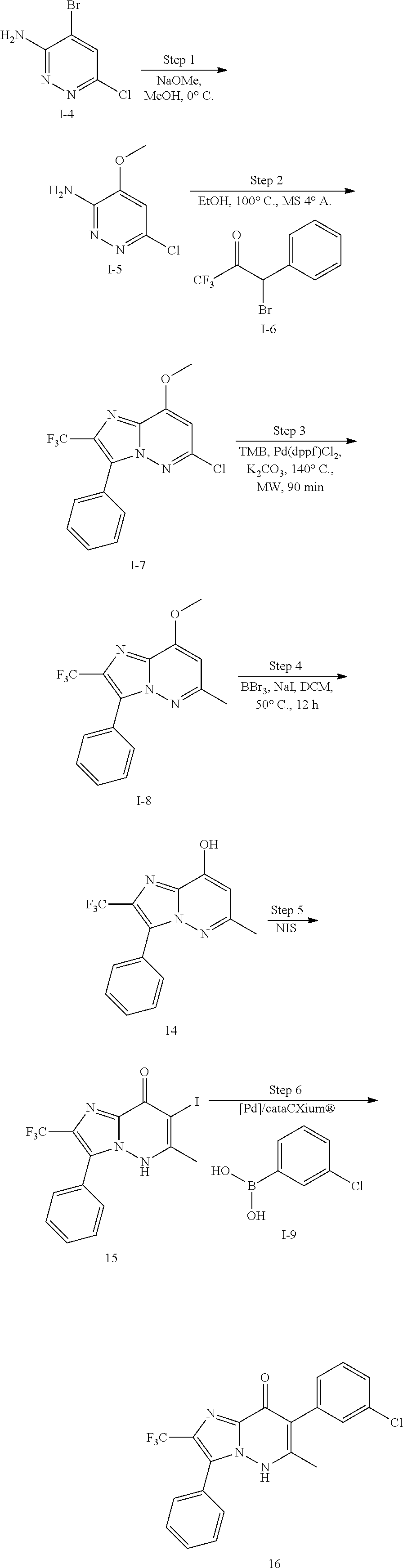

### Step 1

Synthesis of 6-chloro-4-methoxypyridazin-3-amine (I-5): To a solution of 4-bromo-6-chloropyridazin-3-amine (I-4, 1.0 g, 4.8 mmol, Combi-Blocks, San Diego, Calif.) in methanol (5 mL) was added sodium methoxide (38.38 mL, 0.5M in MeOH) at 0° C. The ice bath was removed, and the solution stirred for 12 h. After completion, the solution was poured into water (50 mL) then extracted 3× with EtOAc. The organic layers were combined, washed with brine, dried over Na₂SO₄, then concentrated in vacuo to afford 1-5, which was used in the next step without further purification (667 mg, 4.18 mmol, 87% yield). LC-MS (ESI): 99.4% purity, m/z=160.2 [M+H]. ¹H NMR (400 MHz, Methanol-d₄) δ 7.00 (s, 1H), 4.00 (s, 3H).

### Step 2

Synthesis of 6-chloro-8-methoxy-3-phenyl-2-(trifluoromethyl)imidazo[1,2-b]pyridazine (1-7): Compound I-5 (1.15 g, 7.22 mmol, 1.2 equiv.) and 3-bromo-1,1,1-trifluoro-3-phenylpropan-2-one (1-6, 1.6 g, 6.01 mmol, 1 equiv., Enamine, Monmouth, N.J.) were dissolved in EtOH (12.03 mL) with 4A° molecular sieves in a sealed microwave vial. The reaction mixture was immediately heated to 90° C., and left to stir for 18 h. After cooling to room temperature, the solvent was removed in vacuo and the crude material was purified by Teledyne ISCO Combi-flash (heptane/EtOAc), to afford 1-7 as a white solid (756 mg, 2.31 mmol, 38% yield). LC-MS (ESI): 97% purity, m/z=328.15 [M+H]. ¹H NMR (400 MHz, Methanol-d₄) δ 7.59-7.57 (m, 5H), 7.00 (s, 1H), 4.21 (s, 3H).

### Step 3

Synthesis of 8-methoxy-6-methyl-3-phenyl-2-(trifluoromethyl)imidazo[1,2-b]pyridazine (1-8): Compound I-7 (300 mg, 0.9155 mmol, 1 equiv.), trimethylboroxine (TMB, 197 μL, 1.2 equiv.), Pd(dppf)Cl₂ (100.5 mg, 0.1373 mmol, 15 mol %), and potassium carbonate (379 mg, 2.7465 mmol, 3 equiv.) were suspended in degassed 1,4-dioxane (18.31 mL, 0.05 M) and irradiated in a microwave apparatus at 140° C., for 1.5 h. Upon completion, the resulting mixture was filtered, and the solvent evaporated in vacuo. The crude material was purified by Teledyne ISCO Combi-Flash (heptane/EtOAc) to give I-8 (83.0 mg, 0.2701 mmol, 29.5%). LC-MS (ESI): 91% purity, m/z=308.25 [M+H]. ¹H NMR (400 MHz, Methanol-d₄) δ 7.60-7.54 (m, 5H), 6.78 (s, 1H), 4.16 (s, 3H), 2.51 (s, 3H).

### Step 4

Synthesis of 6-methyl-3-phenyl-2-(trifluoromethyl)imidazo[1,2-b]pyridazin-8-ol (14): Compound I-8 (70 mg, 0.2278 mmol) was dissolved in DCM (0.9113 mL, 0.25 M) under N₂, the mixture was cooled down to 0° C., and boron tribromide (BBr₃) (1.82 mL, 1.82 mmol, 1M in DCM) was added dropwise. The cooling bath was removed, and the reaction was left to stir for 30 minutes before the portion-wise addition of sodium iodide (68.3 mg, 0.4556 mmol, 2 equiv.) then heated to 50° C., for 24 h. Once complete, the mixture was cooled to 0° C., before the dropwise addition of methanol (5 mL). The mixture was then concentrated in vacuo before being dissolved in DCM, then washed sequentially with water, brine, dried over Na₂SO₄, filtered, and concentrated in vacuo to afford Compound 14 as a yellow solid (59 mg, 0.2012 mmol, 88% yield). LC-MS (ESI): 97% purity, m/z=294.25 [M+H].

### Step 5

Synthesis of 7-iodo-6-methyl-3-phenyl-2-(trifluoromethyl)imidazo[1,2-b]pyridazin-8(5H)-one (15): Compound 14 (137 mg, 0.4672 mmol, 1 equiv.) was suspended in DMF (1.87 mL) and cooled to 0° C. N-iodosuccinimide (115.6 mg, 0.5139 mmol, 1.1 equiv.) was added in portionwise. After 15 min of stirring at 0° C., 10 mL water was added, and the mixture was transferred to a separatory funnel. The aqueous layer was extracted 3× with EtOAc, and the combined organic layers were successively washed with sodium thiosulfate (5%), brine, dried over Na₂SO₄, filtered, and concentrated in vacuo to afford the crude Compound 15 as a yellow solid (149 mg, 0.3565 mmol, 76.3% yield). LC-MS (ESI); 92% purity m/z=419.75 [M+H].

### Step 6

Synthesis of 7-(3-chlorophenyl)-6-methyl-3-phenyl-2-(trifluoromethyl)imidazo[1,2-b]pyridazin-8(5H)-one (16): Compound 15 (16.5 mg, 0.039 mmol, 1.0 equiv.), (3-chlorophenyl)boronic acid (1-9, 15.7 mg, 0.10 mmol, 1.5 equiv., TCI America, Portland, Oreg.), palladium acetate (0.88 mg, 0.0039 mol, 10 mol %), cataCXium® A (2.8 mg, 0.0079 mmol, 20 mol %, Sigma-Aldrich) and cesium carbonate (32 mg, 0.098 mmol, 2.5 equiv.) were added to a sealed vial. After three vacuum/N₂ cycles, degassed dioxane (262 μL) and water (132 μL) were added. The vial was capped under N₂ and heated to 100° C., for 2 h. The reaction was diluted with EtOAc and poured on saturated NH₄Cl (aq). The layers were separated and the aqueous layer was extracted 3× with EtOAc. The combined organic layers were washed with brine, dried over Na₂SO₄, filtered, and concentrated in vacuo. The crude material was purified by Teledyne ISCO Combi-Flash (heptane/EtOAc). The fractions containing the desired compound were concentrated, then lyophilized, to give Compound 16 as a white solid (4.1 mg, 0.010 mmol, 26% yield). LC-MS: 99.7% purity, m/z=401.9 [M+H]. ¹H NMR (400 MHz, DMSO-d6) δ 7.67-7.42 (m, 8H), 7.33 (d, J=6.9 Hz, 1H), 2.15 (s, 3H).

Additional compounds 18-22 were prepared by replacing (3-chlorophenyl)boronic acid 1-9 with a suitable boronic acid compound (commercially available, e.g. Combi-Blocks (San Diego, Calif.). TCI America (Portland, Oreg.), Alfa Aesar (Tewksbury. Mass.), Matrix Scientific (Columbia, S.C.)).
7-(3,5-difluorophenyl)-6-methyl-3-phenyl-2-(trifluoromethyl)imidazo[1,2-b]pyridazin-8(5H)-one (18);
7-(3-methoxyphenyl)-6-methyl-3-phenyl-2-(trifluoromethyl)imidazo[1,2-b]pyridazin-8(5H)-one (19);
7-(3-bromophenyl)-6-methyl-3-phenyl-2-(trifluoromethyl)imidazo[1,2-b]pyridazin-8(5H)-one (20);
7-(4-ethylphenyl)-6-methyl-3-phenyl-2-(trifluoromethyl)imidazo[1,2-b]pyridazin-8(5H)-one (21); and 7-(1H-indol-5-yl)-6-methyl-3-phenyl-2-(trifluoromethyl)imidazo[1,2-b]pyridazin-8(5H)-one (22):

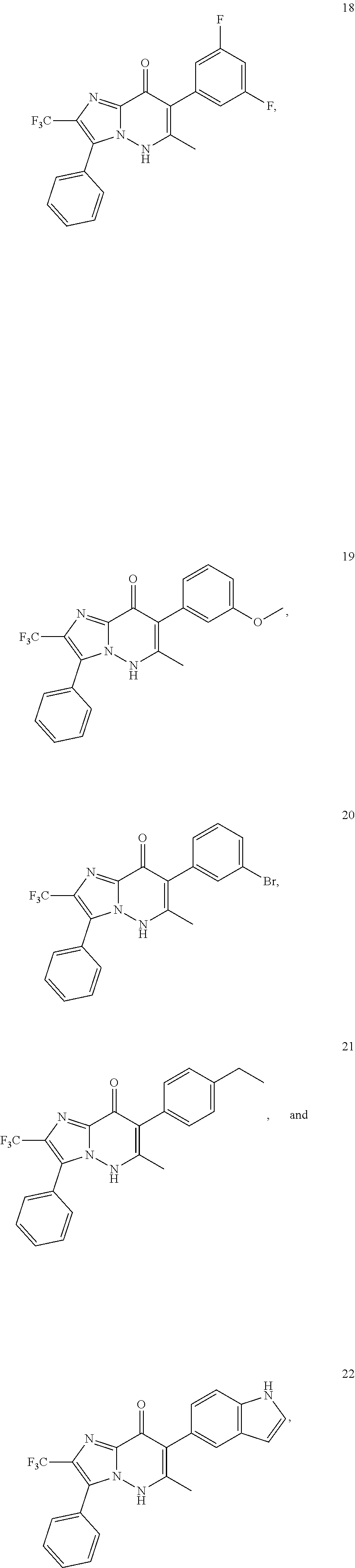 [SMILES: Cc1[nH]n2c(-c3ccccc3)c(C(F)(F)F)nc2c(=O)c1-c1cc(F)cc(F)c1 ; COc1cccc(-c2c(C)[nH]n3c(-c4ccccc4)c(C(F)(F)F)nc3c2=O)c1 ; Cc1[nH]n2c(-c3ccccc3)c(C(F)(F)F)nc2c(=O)c1-c1cccc(Br)c1 ; CCc1ccc(-c2c(C)[nH]n3c(-c4ccccc4)c(C(F)(F)F)nc3c2=O)cc1 ; Cc1[nH]n2c(-c3ccccc3)c(C(F)(F)F)nc2c(=O)c1-c1ccc2[nH]ccc2c1]

were prepared by this method, using the starting boronic acid as shown in the following Table 1.

TABLE 1
Additional compounds prepared by the methods of Example 3.

| Comp. # | Boronic Acid | ¹H NMR | Purity (%) | MS (m/z) |
|---|---|---|---|---|
| 18 | 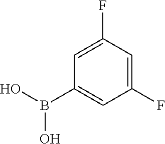 [SMILES: OB(O)c1cc(F)cc(F)c1] | ¹H NMR (400 MHz, Methanol- d₄) δ 7.63 (s, 2H), 7.55 (d, J = 6.6 Hz, 3H), 7.04 (d, J = 7.8 Hz, 3H), 2.25 (s, 3H). | 95.8 | 406.25 |
| 19 | 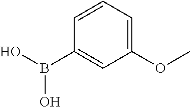 | not purified | | |
| 20 | 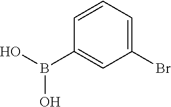 | not purified | | |
| 21 | 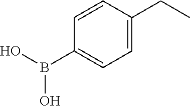 | ¹H NMR (400 MHz, Methanol- d₄) δ 7.78-7.49 (m, 5H), 7.37 (d, J = 7.7 Hz, 2H), 7.28 (d, J = 7.9 Hz, 2H), 2.75 (q, J = 7.6 Hz, 2H), 2.21 (s, 3H), 1.37-1.27 (d, 3H). | 91.0 | 398.30 |
| 22 | 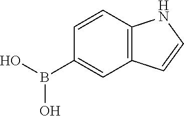 [SMILES: OB(O)c1ccc2[nH]ccc2c1] | ¹H NMR (400 MHz, Methanol- d₄) δ 7.66 (d, J = 7.3 Hz, 2H), 7.60-7.50 (m, 5H), 7.33 (d, J = 2.7 Hz, 1H), 7.07 (d, J = 8.4 Hz, 1H), 6.54 (d, J = 2.8 Hz, 1H), 2.22 (s, 3H) | 91.3 | 409.25 |

### Example 4

Synthesis of compounds where R¹ is —C(═O)NR^{a}R^{b} were carried out in one step as follows:

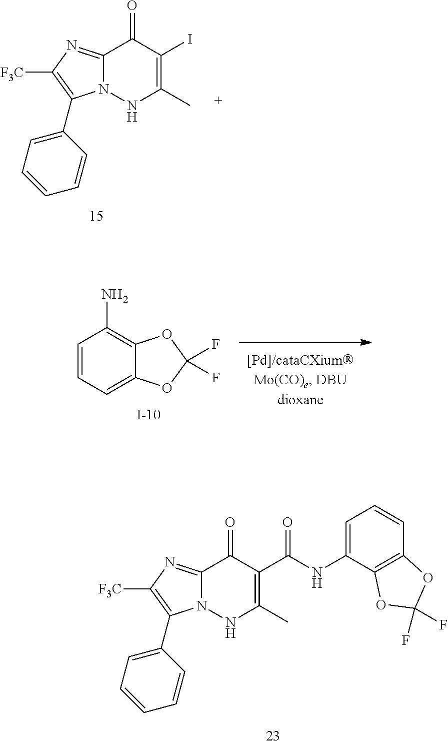

Synthesis of N-(2,2-difluorobenzo[d][1,3]dioxol-4-yl)-6-methyl-8-oxo-3-phenyl-2-(trifluoromethyl)-5,8-dihydroimidazo[1,2-b]pyridazine-7-carboxamide (23): Compound 15 (43 mg, 0.10 mmol, 1.0 equiv., see Example 3), 2,2-difluoro-1,3-benzodioxol-4-amine (I-10, 58 μL, 0.51 mmol, 5 equiv., Alfa Aesar. Tewksbury, Mass.), palladium acetate (2.4 mg, 0.010 mmol, 10 mol %), cataCXium® A (7.4 mg, 0.021 mmol, 20 mol %), and molybdenum hexacarbonyl (10 mg, 0.038 mmol, 0.37 equiv.) in dioxane (0.50 mL) were mixed in a microwave vial. After purging with N₂, DBU (46 μL, 0.31 mmol, 3 equiv.) was added in one portion to the reaction vial and purged again. The vial was capped and heated to 90° C., for 3 h. Upon completion, CH₂Cl₂ was added to the mixture, which was filtered through Celite. The solvent was evaporated under reduced pressure and the mixture was extracted 3× with EtOAc. The combined organic layers were washed with brine, dried over Na₂SO₄, filtered, and concentrated in vacuo. The crude material was purified by reverse phase flash chromatography (C18 column) using a gradient of MeCN in water (+0.1% formic acid). The fractions containing the desired compound were partially concentrated, then lyophilized, to give compound 23 as an off-white solid (29 mg, 0.059 mmol, 57% yield). LC-MS: 98.2% purity, m/z=493.20 [M+H]. ¹H NMR (400 MHz, DMSO-d6) δ 14.06 (s, 1H), 8.19 (d, J=8.5 Hz, 1H), 7.60-7.56 (m, 2H), 7.55-7.50 (m, 2H), 7.50-7.45 (m, 1H), 7.21 (s, 1H), 7.13 (t, J=8.3 Hz, 1H), 7.02 (d, J=8.0 Hz, 1H), 2.57 (s, 3H).

The compound 6-methyl-8-oxo-3-phenyl-2-(trifluoromethyl)-5,8-dihydroimidazo[1,2-b]pyridazine-7-carboxylic acid (24),

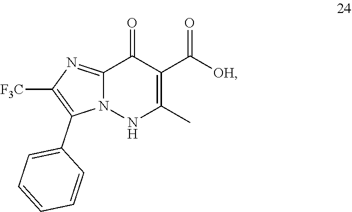 [SMILES: Cc1[nH]n2c(-c3ccccc3)c(C(F)(F)F)nc2c(=O)c1C(=O)O]

was isolated as a by-product of this reaction. LC-MS: 90.0% purity, m/z=337.90 [M+H]. ¹H NMR (400 MHz, Methanol-d₄) δ 7.67-7.50 (m, 5H), 2.68 (s, 3H).

Additional compounds 25-45 were prepared by replacing 2,2-difluoro-1,3-benzodioxol-4-amine I-10 with a suitable amine compound (amine starting materials were commercially available, e.g. Combi-Blocks (San Diego, Calif.), Synthonix (Wake Forest, N.C.); Enamine (Monmouth, N.J.), Ambeed (Arlington Heights, Ill.), Aurum Pharmtech (Franklin Park, N.J.)), and optionally replacing the 7-iodo compound 15 with the 7-iodo compound 4 (see Example 1).
N-(2-cyanophenyl)-6-methyl-8-oxo-3-phenyl-2-(trifluoromethyl)-5,8-dihydroimidazo[1,2-b]pyridazine-7-carboxamide (25);
N-(5-chloro-2-cyanophenyl)-6-methyl-8-oxo-3-phenyl-2-(trifluoromethyl)-5,8-dihydroimidazo[1,2-b]pyridazine-7-carboxamide (26);
N-(3-cyanopyridin-4-yl)-6-methyl-8-oxo-3-phenyl-2-(trifluoromethyl)-5,8-dihydroimidazo[1,2-b]pyridazine-7-carboxamide (27);
N-(2-cyano-5-fluorophenyl)-6-methyl-8-oxo-3-phenyl-2-(trifluoromethyl)-5,8-dihydroimidazo[1,2-b]pyridazine-7-carboxamide (28);
N-(2-cyano-5-fluoropyridin-3-yl)-6-methyl-8-oxo-3-phenyl-2-(trifluoromethyl)-5,8-dihydroimidazo[1,2-b]pyridazine-7-carboxamide (29);
N-(2-cyano-4-fluorophenyl)-6-methyl-8-oxo-3-phenyl-2-(trifluoromethyl)-5,8-dihydroimidazo[1,2-b]pyridazine-7-carboxamide (30);
N-(2-cyano-3-fluorophenyl)-6-methyl-8-oxo-3-phenyl-2-(trifluoromethyl)-5,8-dihydroimidazo[1,2-b]pyridazine-7-carboxamide (31);
N-(4-cyanopyridin-3-yl)-6-methyl-8-oxo-3-phenyl-2-(trifluoromethyl)-5,8-dihydroimidazo[1,2-b]pyridazine-7-carboxamide (32);
6-methyl-8-oxo-3-phenyl-2-(trifluoromethyl)-N-(4-(trifluoromethyl)thiazol-2-yl)-5,8-dihydroimidazo[1,2-b]pyridazine-7-carboxamide (33);
2-cyano-N-(2-cyano-5-fluoropyridin-3-yl)-6-methyl-8-oxo-3-phenyl-5,8-dihydroimidazo[1,2-b]pyridazine-7-carboxamide (34);
2-cyano-N-(2-cyanopyridin-3-yl)-6-methyl-8-oxo-3-phenyl-5,8-dihydroimidazo[1,2-b]pyridazine-7-carboxamide (35);
2-cyano-N-(3-cyanopyridin-4-yl)-6-methyl-8-oxo-3-phenyl-5,8-dihydroimidazo[1,2-b]pyridazine-7-carboxamide (36);
2-cyano-6-methyl-8-oxo-3-phenyl-N-(2-(trifluoromethyl)thiazol-4-yl)-5,8-dihydroimidazo[1,2-b]pyridazine-7-carboxamide (37);
N-(5-chloro-2-cyanophenyl)-2-cyano-6-methyl-8-oxo-3-phenyl-5,8-dihydroimidazo[1,2-b]pyridazine-7-carboxamide (38);
2-cyano-N-(2-cyano-5-fluorophenyl)-6-methyl-8-oxo-3-phenyl-5,8-dihydroimidazo[1,2-b]pyridazine-7-carboxamide (39);
2-cyano-N-(2-cyano-3-fluorophenyl)-6-methyl-8-oxo-3-phenyl-5,8-dihydroimidazo[1,2-b]pyridazine-7-carboxamide (40);
2-cyano-N-(2-cyano-4-fluorophenyl)-6-methyl-8-oxo-3-phenyl-5,8-dihydroimidazo[1,2-b]pyridazine-7-carboxamide (41); 2-cyano-6-methyl-8-oxo-3-phenyl-N-(6-(trifluoromethyl)pyridin-2-yl)-5,8-dihydroimidazo[1,2-b]pyridazine-7-carboxamide (42);
2-cyano-N-(3-cyanothiophen-2-yl)-6-methyl-8-oxo-3-phenyl-5,8-dihydroimidazo[1,2-b]pyridazine-7-carboxamide (43);
N-(4-chloro-2-cyanophenyl)-2-cyano-6-methyl-8-oxo-3-phenyl-5,8-dihydroimidazo[1,2-b]pyridazine-7-carboxamide (44); and
2-cyano 1N-(3-cyanopyridazin-4-yl)-6-methyl-8-oxo-3-phenyl-5,8-dihydroimidazo[1,2-b]pyridazine-7-carboxamide (45):

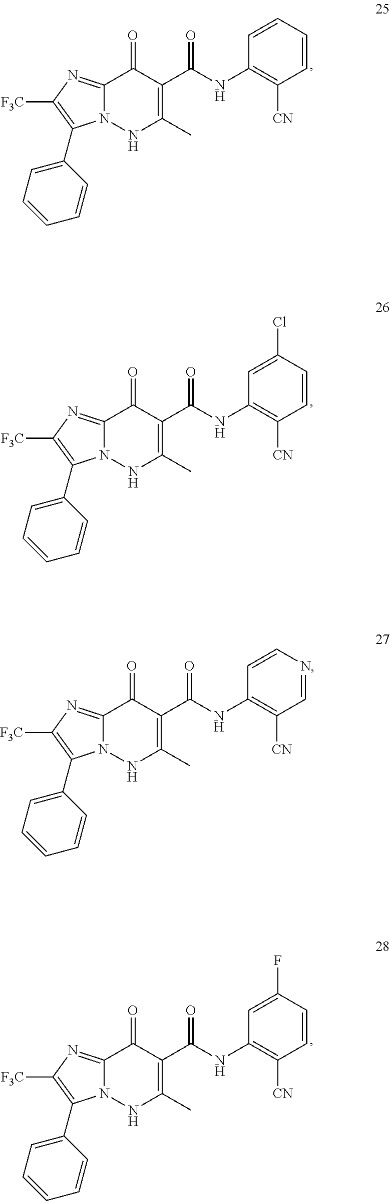 [SMILES: Cc1[nH]n2c(-c3ccccc3)c(C(F)(F)F)nc2c(=O)c1C(=O)Nc1ccccc1C#N ; Cc1[nH]n2c(-c3ccccc3)c(C(F)(F)F)nc2c(=O)c1C(=O)Nc1cc(Cl)ccc1C#N ; Cc1[nH]n2c(-c3ccccc3)c(C(F)(F)F)nc2c(=O)c1C(=O)Nc1ccncc1C#N ; Cc1[nH]n2c(-c3ccccc3)c(C(F)(F)F)nc2c(=O)c1C(=O)Nc1cc(F)ccc1C#N]

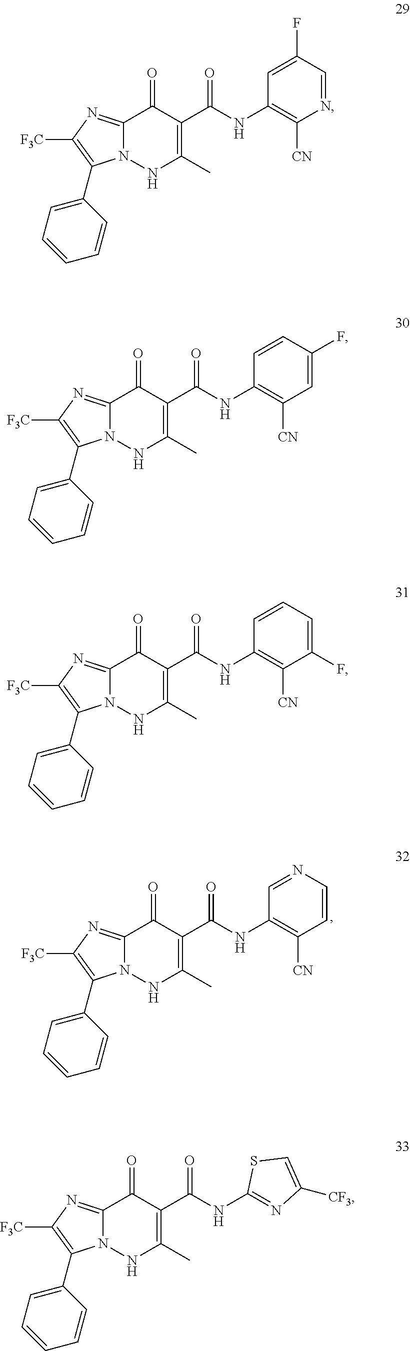 [SMILES: Cc1[nH]n2c(-c3ccccc3)c(C(F)(F)F)nc2c(=O)c1C(=O)Nc1cc(F)cnc1C#N ; Cc1[nH]n2c(-c3ccccc3)c(C(F)(F)F)nc2c(=O)c1C(=O)Nc1ccc(F)cc1C#N ; Cc1[nH]n2c(-c3ccccc3)c(C(F)(F)F)nc2c(=O)c1C(=O)Nc1cccc(F)c1C#N ; Cc1[nH]n2c(-c3ccccc3)c(C(F)(F)F)nc2c(=O)c1C(=O)Nc1cnccc1C#N ; Cc1[nH]n2c(-c3ccccc3)c(C(F)(F)F)nc2c(=O)c1C(=O)Nc1nc(C(F)(F)F)cs1]

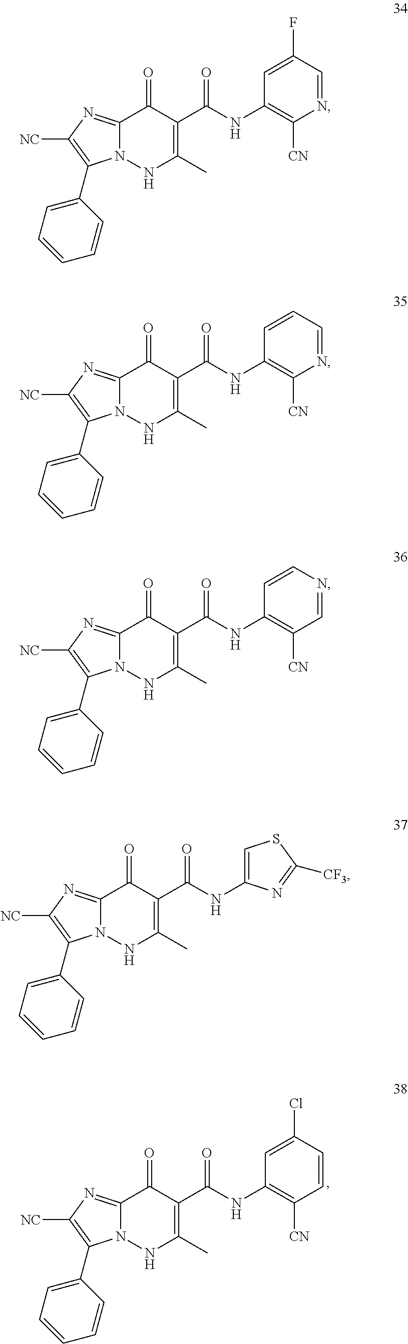 [SMILES: Cc1[nH]n2c(-c3ccccc3)c(C#N)nc2c(=O)c1C(=O)Nc1cc(F)cnc1C#N ; Cc1[nH]n2c(-c3ccccc3)c(C#N)nc2c(=O)c1C(=O)Nc1cccnc1C#N ; Cc1[nH]n2c(-c3ccccc3)c(C#N)nc2c(=O)c1C(=O)Nc1ccncc1C#N ; Cc1[nH]n2c(-c3ccccc3)c(C#N)nc2c(=O)c1C(=O)Nc1csc(C(F)(F)F)n1 ; Cc1[nH]n2c(-c3ccccc3)c(C#N)nc2c(=O)c1C(=O)Nc1cc(Cl)ccc1C#N]

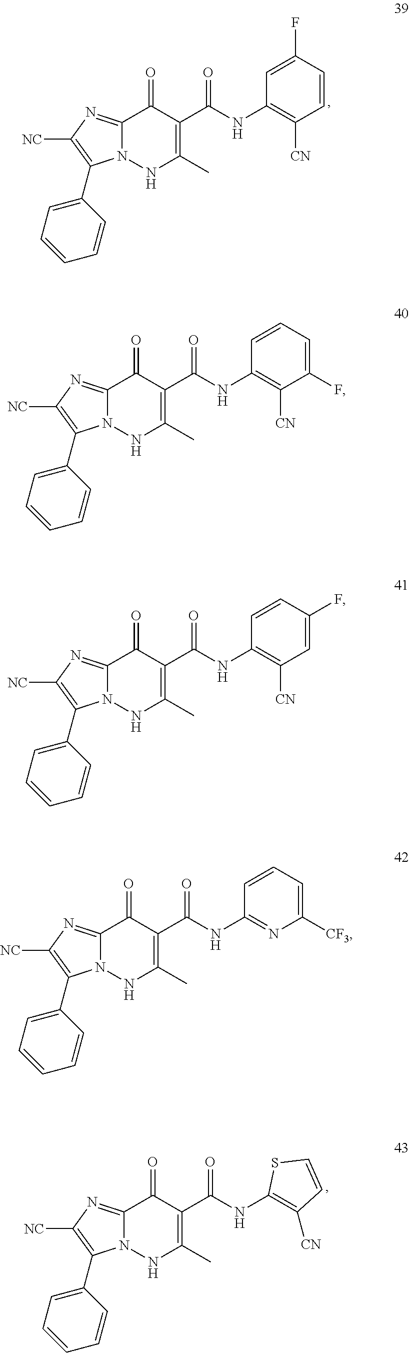 [SMILES: Cc1[nH]n2c(-c3ccccc3)c(C#N)nc2c(=O)c1C(=O)Nc1cc(F)ccc1C#N ; Cc1[nH]n2c(-c3ccccc3)c(C#N)nc2c(=O)c1C(=O)Nc1cccc(F)c1C#N ; Cc1[nH]n2c(-c3ccccc3)c(C#N)nc2c(=O)c1C(=O)Nc1ccc(F)cc1C#N ; Cc1[nH]n2c(-c3ccccc3)c(C#N)nc2c(=O)c1C(=O)Nc1cccc(C(F)(F)F)n1 ; Cc1[nH]n2c(-c3ccccc3)c(C#N)nc2c(=O)c1C(=O)Nc1sccc1C#N]

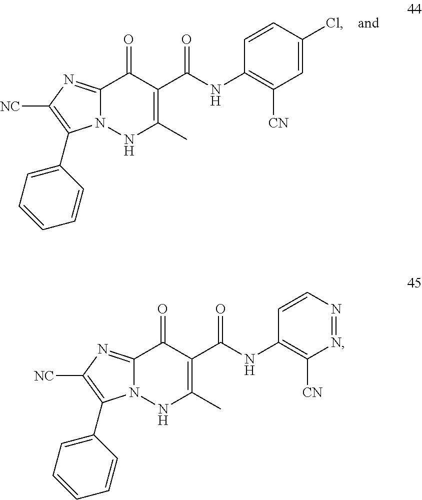 [SMILES: Cc1[nH]n2c(-c3ccccc3)c(C#N)nc2c(=O)c1C(=O)Nc1ccc(Cl)cc1C#N ; "and" ; Cc1[nH]n2c(-c3ccccc3)c(C#N)nc2c(=O)c1C(=O)Nc1ccnnc1C#N]

were prepared by this method, using the starting iodo compound and amine compound (Iodo/Amine) as shown in the following Table 2.

TABLE 2
Additional compounds prepared by the methods of Example 4.

| Comp. # | Iodo/Amine | ¹H NMR | Purity (%) | MS (m/z) |
|---|---|---|---|---|
| 25 | Comp. 15/ 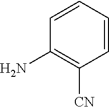 | ¹H NMR (400 MHz, Methanol-d₄) δ 8.39-8.27 (m, 1H), 7.69 (d, J = 7.8 Hz, 1H), 7.52 (q, J = 7.4, 6.8 Hz, 6H), 7.21 (t, J = 7.6 Hz, 1H), 2.69 (s, 3H). | 92.7 | 438.20 |
| 26 | Comp. 15/ 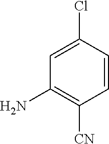 | ¹H NMR (400 MHz, Methanol-d₄) δ 8.63 (s, 1H), 7.64 (dd, J = 17.8, 7.8 Hz, 3H), 7.56-7.47 (m, 3H), 7.19 (d, J = 8.4 Hz, 1H), 2.68 (s, 3H). | 94.1 | 472.25 |
| 27 | Comp. 15/ 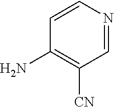 | ¹H NMR (400 MHz, Methanol-d₄) δ 8.73 (s, 1H), 8.68 (d, J = 6.1 Hz, 1H) 8.55 (d, J = 6.0 Hz, 1H), 7.67 7.48 (m, 5H), 2.68 (s, 3H). | 98.3 | 439.20 |
| 28 | Comp. 15/ 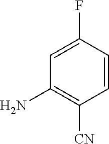 | ¹H NMR (400 MHz, Methanol-d₄) δ 8.40 (d, J = 13.3 Hz, 1H), 7.78- 7.69 (m, 1H), 7.56 (dt, J = 34.3, 6.8 Hz, 5H), 6.98-6.88 (m, 1H), 2.69 (s, 3H). | 91.5 | 456.25 |
| 29 | Comp. 15/ 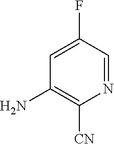 | ¹H NMR (400 MHz Methanol-d₄) δ 8.86 (d, J = 11.6 Hz, 1H), 8.25 (d, J = 2.0 Hz, 1H), 7.62 (d, J = 7.2 Hz, 2H), 7.58-7.22 (m, 3H), 2.67 (s, 3H). | 98.6 | 457.15 |
| 30 | Comp. 15/ 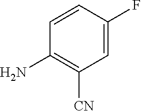 | ¹H NMR (400 MHz, Methanol-d₄) δ 8.16 (s, 1H), 7.66-7.45 (m, 6H), 7.39 (s, 1H), 2.67 (s, 3H). | 98.7 | 456.25 |
| 31 | Comp. 15/ 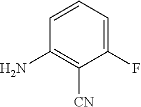 | ¹H NMR (400 MHz, Methanol-d₄) δ 8.13 (d, J = 8.3 Hz, 1H), 7.74- 7.36 (m, 5H), 6.98 (t, J = 8.6 Hz, 2H), 2.65 (s, 3H). | 96.1 | 456.25 |
| 32 | Comp. 15/ 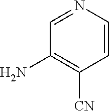 | ¹H NMR (400 MHz, Methanol-d₄) δ 9.61 (s, 1H), 8.39 (d, J = 4.8 Hz, 1H), 7.70 (d, J = 4.9 Hz, 1H), 7.63 (d, J = 6.7 Hz, 2H), 7.53 (d, J = 7.2 Hz, 3H), 2.68 (s, 3H). | 99.7 | 439.25 |
| 33 | Comp. 15/ 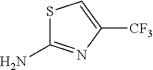 | ¹H NMR (400 MHz, Methanol-d₄) δ 7.66-7.46 (m, 6H), 2.70 (s, 3H). | 94.4 | 488.25 |
| 34 | Comp. 4/ 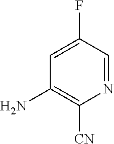 | ¹H NMR (400 MHz, DMSO) δ 8.89 (dd, J = 11.8, 2.6 Hz, 1H), 8.39 (d, J = 2.6 Hz, 1H), 7.98- 7.94 (m, 2H), 7.65-7.57 (m, 2H), 7.56-7.48 (m, 1H), 3.04 (s, 3H). | 97.8 | 412.3 |
| 35 | Comp. 4/ 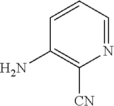 | ¹H NMR (400 MHz, DMSO) δ 14.56 (s, 1H), 8.92 (dd, J = 8.7, 1.3 Hz, 1H), 8.34 (dd, J = 4.4, 1.4 Hz, 1H), 7.99-7.93 (m, 2H), 7.64 (dd, J = 8.7, 4.5 Hz, 1H), 7.59 (t, J = 7.6 Hz, 2H), 7.51 (t, J = 7.4 Hz, 1H), 2.64 (s, 3H). | 99.5 | 394.4 (M − H) |
| 36 | Comp. 4/ 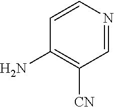 | ¹H NMR (500 MHz, DMSO) δ 14.89 (s, 1H), 8.84 (s, 1H), 8.62 (d, J = 6.2 Hz, 1H), 8.59 (d, J = 6.1 Hz, 1H), 7.98-7.93 (m, 2H), 7.59 (t, J = 7.6 Hz. 2H), 7.51 (t, J = 7.4 Hz, 1H), 2.63 (s, 3H). | 98.8 | 394.3 (M − H) |
| 37 | Comp. 4/ 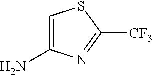 | ¹H NMR (500 MHz, DMSO) δ 15.33 (s, 1H), 7.97-7.92 (m, 2H), 7.84 (s, 1H), 7.63-7.56 (m, 2H), 7.55-7.48 (m, 1H), 2.65 (s, 3H). | 100.0 | 443.3 |
| 38 | Comp. 4/ 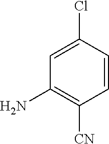 | ¹H NMR (500 MHz, DMSO) δ 14.41 (s, 1H), 8.58 (d, J = 9.1 Hz, 1H), 7.96-7.93 (m, 2H), 7.89 (d, J = 2.6 Hz, 1H), 7.66 (dd, J = 9.1, 2.7 Hz, 1H), 7.60-7.56 (m, 2H), 7.50 (t, J = 7.5 Hz, 1H), 2.62 (s, 3H). | 97.5 | 427.3 (M − H) |
| 39 | Comp, 4/ 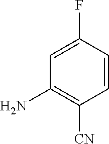 | ¹H NMR (500 MHz, DMSO) δ 14.59 (s, 1H), 8.48 (dd, J = 12.6, 2.6 Hz, 1H), 7.97 (dd, J = 8.3, 1.2 Hz, 2H), 7.85 (dd, J = 8.7, 6.4 Hz, 1H), 7.60 (t, J = 7.6 Hz, 2H), 7.52 (t, J = 7.4 Hz, 1H), 7.04-6.97 (m, 1H), 2.64 (s, 3H). | 98.5 | 411.2 (M − H) |
| 40 | Comp. 4/ 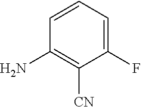 | ¹H NMR (500 MHz, DMSO) δ 14.50 (s, 1H), 8.41 (d, J = 8.7 Hz, 1H), 7.98-7.93 (m, 2H), 7.65 (dd, J = 15.8, 8.4 Hz, 1H), 7.59 (t, J = 7.6 Hz, 2H), 7.51 (t, J = 7.5 Hz, 1H), 7.07 (t, J = 9.2 Hz, 1H), 2.63 (s, 3H). | 98.3 | 411.3 (M − H) |
| 41 | Comp. 4/ 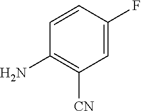 | ¹H NMR (500 MHz, DMSO) δ 14.36 (s, 1H), 8.60 (dd, J = 9.4, 5.2 Hz, 1H) 8.11-8.04 (m, 2H), 7.85 (dd, J = 8.5, 3.1 Hz, 1H), 7.70 (t, J = 7.6 Hz, 2H), 7.65-7.56 (m, 2H), 2.74 (s, 3H). | 100.0 | 411.3 (M − H) |
| 42 | Comp. 4/ 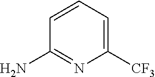 [SMILES: Nc1cccc(C(F)(F)F)n1] | ¹H NMR (500 MHz, DMSO) δ 14.08 (s, 1H), 8.59-8.55 (m, 1H), 8.02-7.94 (m, 3H), 7.62-7.58 (m, 2H), 7.53-7.47 (m, 2H), 2.67- 2.62 (s, 3H). | 100.0 | 438.4 |
| 43 | Comp. 4/ 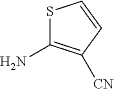 | ¹H NMR (500 MHz, DMSO) δ 15.69 (s, 1H), 7.98-7.94 (m, 2H), 7.62-7.58 (m, 2H), 7.55-7.50 (m, 1H) 7.16-7.13 (m, 1H), 7.04- 7.01 (m, 1H), 2.65 (s, 3H). | 100.0 | 400.4 |
| 44 | Comp. 4/ 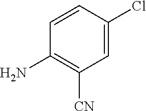 | 1H NMR (500 MHz, DMSO) δ 14.41 (s, 1H), 8.57 (d, J = 9.2 Hz, 1H), 7.95 (d, J = 7.1 Hz, 2H), 7.89 (d, J = 2.6 Hz, 1H), 7.66 (dd, J = 9.2, 2.5 Hz, 1H), 7.58 (t, J = 7.6 Hz, 2H), 7.50 (d, J = 7.4 Hz, 1H), 2.62 (s, 3H). | 99.5 | 427.5 |
| 45 | Comp. 4/ 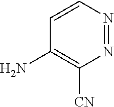 | ¹H NMR (500 MHz, CD₃CN) δ 15.19-15.12 (m, 1H), 9.00 (s, 1H), 8.82 (s, 1H), 8.01-7.95 (m, 2H), 7.60-7.54 (m, 2H), 7.50 (s, 2H), 2.67 (s, 3H). | 100.0 | 396.4 |

### Example 5: cGAS Biochemical Activity Assay

Human cGAS sequence encoding amino acids 155-522 was cloned into a pET (EMD Millipore) based expression plasmid. The resulting construct contained a tandem N-terminal hexahistidine tag, maltose binding protein fusion followed by a tobacco etch virus protease cleavable linker preceding cGAS amino acids 155-522.

Construct sequence: Amino acids 155-522,
Human cGAS
SEQ. ID No. 1
DAAPGASKLRAVLEKLKLSRDDISTAAGMVKGVVDHLLLRLKCDSAFRGV
GLLNTGSYYEHVKISAPNEFDVMFKLEVPRIQLEEYSNTRAYYFVKFKRN
PKENPLSQFLEGEILSASKMLSKFRKIIKEEINDIKDTDVIMKRKRGGSP
AVTLLISEKISVDITLALESKSSWPASTQEGLRIQNWLSAKVRKQLRLKP
FYLVPKHAKEGNGFQEETWRLSFSHIEKEILNNHGKSKTCCENKEEKCCR
KDCLKLMKYLLEQLKERFKDKKHLDKFSSYHVKTAFFHVCTQNPQDSQWD
RKDLGLCFDNCVTYFLQCLRTEKLENYFIPEFNLFSSNLIDKRSKEFLTK
QIEYERNNEFPVFDEF,

Protein was expressed and purified from E. coli BL21 DE3 Rosetta 2 (EMD Millipore) cells using standard techniques. Cells were grown in 2× yeast extract tryptone medium and expression was initiated via the addition of isopropyl β-D-1-thiogalactopyranoside. Expression proceeded overnight at 18° C. Cells were harvested by centrifugation and subsequently used via sonication. Insoluble fraction was removed by centrifugation. Maltose binding protein (MBP) fusion proteins were purified on a dextrin sepharose column (GE Healthcare) and the MBP tag was removed using tobacco etch virus protease overnight during dialysis. Protein was further purified on a heparin column (GE Healthcare) and eluted using a NaCl gradient. Column fraction were pooled and further purified on a Superdex 75 gel filtration column (GE Healthcare). Protein was quantified using 280 nm absorbance. Protein was then flash frozen in liquid nitrogen and stored at −80° C., until use.

Potential antagonists were diluted in 100% dimethyl sulfoxide and added to the reaction. Final dimethyl sulfoxide concentration was 5%. The compounds were tested from 250 μM with 5-fold serial dilutions down to 0.000128 μM.

Two complementary DNA oligos (IDT DNA) were annealed by slow cooling from 95° C. The resulting double stranded DNA was used to activate cGAS.

Top strand oligo:
SEQ. ID No. 2
5′-TACAGATCTACTAGTGATCTATGACTGATCTGTACATGATCTAC
A-3′
Bottom strand oligo:
SEQ. ID No. 3
3′-TGTAGATCATGTACAGATCAGTCATAGATCACTAGTAGATCTGT
A-3′

Reactions were performed at 37° C., for 1.25 hours. Reaction buffer: 20 mM Tris HCl pH 9, 100 mM NaCl, 5 mM MgCl₂, 0.1 mg/ml bovine gamma globulin, 1 mM adenosine triphosphate, 250 μM guanosine triphosphate, 0.5 mM Tris(2-carboxyethyl)phosphine hydrochloride, 1 μM double stranded DNA and 25 nM purified cGAS protein.

Reactions were stopped and ATP levels in the reaction were measured using a luciferase based assay. Promega Kinase-Glo Max Assay. Luminescence was measured on a plate reader (Molecular Devices). Values were normalized to control wells lacking compound.

Table 3 below provides IC₅₀ data for certain compounds of the invention on cGAS. “A” indicates an IC₅₀ value of less than 1 μM, “B” indicates an IC₅₀ value between 1 and 50 μM, and “C” indicates an IC₅₀ above the upper limit of the assay (50 μM), or where an IC₅₀ value could not be generated from the data.

TABLE 3

| Compound | cGAS IC₅₀ |
|---|---|
| ID | (μM) |
| 3 | A |
| 5 | A |
| 6 | A |
| 13 | A |
| 16 | |
| 17 | |
| 18 | |
| 21 | |
| 22 | |
| 23 | |
| 24 | |
| 25 | |
| 26 | |
| 27 | |
| 28 | |
| 29 | |
| 30 | |
| 31 | |
| 32 | |
| 33 | |
| 34 | |
| 35 | |
| 36 | |
| 37 | |
| 38 | |
| 39 | |
| 40 | |
| 41 | |
| 42 | |
| 43 | |
| 44 | |
| 45 | |

### Example 6: THP1 Cell-Based cGAS/STING Pathway Activity Assay

A cellular assay can be used to assess the compounds of the invention for their ability to inhibit the cGAS/STING pathway. Cells that express a luciferase-based reporter that is linked to IRF-3 activation are used to determine response as a function of compound concentration. Such an assay is described in Vincent et al., Nature Communications 2017, 8(1):750, doi: 10.1038/s41467-017-00833-9. Compounds of the invention were assessed using similar assay methods in a THP1 cell assay to generate IC₅₀ values. Compounds 5, 6 and 13 have an IC₅₀ below 10 μM.

### Example 7: Inhibition of Cytokine Secretion by Trex1-Knockout (KO) Bone Marrow-Derived Macrophages (BMMs)

The inhibition of secreted cytokine is measured in bone marrow-derived macrophages (BMMs) from diseased mice to evaluate the potency of compounds of the invention as described herein. Mice lacking the gene for Trex1 protein (trex1−/− or Trex1-KO) exhibit cGAS/STING pathway-dependent autoimmune and autoinflammatory disease manifestations, including enhanced cytokine secretion by cells. To derive bone marrow macrophages, marrow from the femurs and tibias that are harvested from Trex1-KO mice is cultured in growth media supplemented with macrophage colony-stimulating factor (M-CSF).

Differentiated BMM are harvested and frozen for subsequent experimentation. For treatment with compound of the present disclosure, a frozen stock of Trex1-KO BMM is thawed and 1×10⁵ cells are plated in 96 well format. BMMs are treated with a dilution series of the compound and incubated overnight at 37° C. 5% CO₂, at which point cellular supernatants are harvested and stored at −80° C., for subsequent analysis. The media used for this dilution series is without FBS. Remaining cells are evaluated for viability using Cell Titer Glo 2.0 kit according to the manufacturer instructions. BMM supernatants are evaluated for the secreted cytokine normal T cell expressed and secreted (RANTES/CCL5) or for secreted cytokine monocyte chemoattractant protein 1 (MCP-1/CCL2) using cytometric bead array mouse Flex Set kits (BD Biosciences). Cytokine concentration is calculated from a standard curve and normalized to vehicle (DMSO) treated cells.

### INCORPORATION BY REFERENCE

The disclosure of each of the patent documents and scientific articles referred to herein is incorporated by reference for all purposes.

### EQUIVALENTS

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A compound of Formula (I): 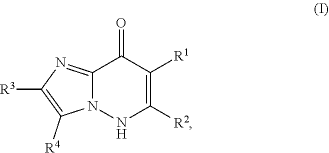
or a pharmaceutically acceptable salt, pharmaceutically acceptable solvate or pharmaceutically acceptable hydrate thereof, wherein
R¹ is Q¹-T¹-(X¹)ₙ;
Q¹ is a bond or C₁₋₃alkylene, wherein the C₁₋₃alkylene group is optionally substituted with one or more substituents independently selected from the group consisting of halo, C₁₋₆alkyl, C₂₋₆alkenyl, C₂₋₆alkynyl, cyano, C₁₋₆haloalkyl, —OR^{w2}, and —NR^{w2}R^{x2};
T¹ is H, halo, cyano, C₃₋₆cycloalkyl, C₆₋₁₀aryl, 3- to 12-membered heterocycloalkyl, 5- to 10-membered heteroaryl, —C(═O)C₀₋₃alkylene-C₃₋₈cycloalkyl, —C(═O)—C₀₋₃alkylene-C₆₋₁₀aryl, —C(═O)—C₀₋₃alkylene-3 to 12-membered heterocycloalkyl, —C(═O)—C₀₋₃alkylene-5 to 10-membered heteroaryl, —C(═O)R^{a}, —C(═O)OR^{a}, —NR^{a}R^{b}, —S(═O)₂R^{a}, —NR^{a}C(═O)R^{a}, —NR^{a}C(═O)NR^{a}R^{b}, —NR^{a}C(═O)OR^{a}, —NR^{a}S(═O)₂R^{a}, —C(═O)NR^{a}S(═O)₂R^{a}, —NR^{a}S(═O)₂NR^{a}R^{b}, —C(═O)NR^{a}R^{b}, or —S(═O)₂NR^{a}R^{b};
each X¹ is independently selected from the group consisting of halo, cyano, oxo, C₀₋₃alkylene-C(═O)R^{c}, C₀₋₃alkylene-OR^{c}, C₀₋₃alkylene-C(═O)OR^{c}, C₀₋₃alkylene-OC(═O)R^{c}, C₀₋₃alkylene-NR^{c}R^{d}, C₀₋₃alkylene-N⁺R^{c}R^{d}R^{d′}, C₀₋₃alkylene-S(═O)ₘR^{c}, C₀₋₃alkylene-NR^{c}C(═O)R^{c}, C₀₋₃alkylene-NR^{c}C(═O)NR^{c}R^{d}, C₀₋₃alkylene-OC(═O)NR^{c}R^{d}, C₀₋₃alkylene-NR^{c}C(═O)OR^{c}, C₀₋₃alkylene-NR^{c}S(═O)₂R^{c}, C₀₋₃alkylene-C(═O)NR^{c}S(═O)₂R, C₀₋₃alkylene-NR^{c}S(═O)NR^{c}R^{d}, C₀₋₃alkylene-C(═O)NR^{c}R^{d}, C₀₋₃alkylene-S(═O)₂NR^{c}R^{d}, C₀₋₃alkylene-C(═NR^{c})NR^{c}R^{d}, C₀₋₃alkylene-NR^{c}C(═NR^{c})NR^{c}R^{d}, and R^{S1}, in which R^{S1} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;
and each R^{S1} is optionally substituted with one or more substituents independently selected from the group consisting of halo, cyano, nitro, oxo, C₁₋₆alkyl, C₂₋₆alkenyl, C₂₋₆alkynyl, C₁₋₆haloalkyl, C₀₋₃alkylene-NR^{e}R^{f}, C₀₋₃alkylene-OR, C₀₋₃alkylene-NR^{e}C(═O)R, C₀₋₃alkylene-NR^{e}C(═O)OR^{e}, C₀₋₃alkylene-NR^{e}C(═O)NR^{e}R^{f}, C₀₋₃alkylene-OC(═O)R^{e}, C₀₋₃alkylene-C(═O)OR^{e}, C₀₋₃alkylene-C(═O)NR^{e}R^{f}, C₀₋₃alkylene-C(═O)R^{e}, C₀₋₃alkylene-S(═O)ₘRe, C₀₋₃alkylene-S(═O)₂NR^{e}R^{f}, C₀₋₃alkylene-NR^{e}S(═O)₂R^{e}, C₀₋₃alkylene-C(═O)NR^{e}S(═O)₂R^{e}, C₀₋₃alkylene-NR^{e}S(═O)₂NR^{e}R^{f}, and R^{S2}, in which R^{S2} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl,
and each R^{S2} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w}, and —NR^{w}R^{x};
R² is Q¹-T²-(X²)ₚ;
Q² is a bond or C₁₋₃alkylene, wherein the C₁₋₃alkylene group is optionally substituted with one or more substituents independently selected from the group consisting of halo, C₁₋₆alkyl, C₂₋₆alkenyl, C₂₋₆alkynyl, cyano, C₁₋₆haloalkyl, —OR^{w3}, and —NR^{w3}R^{x3};
T² is H, halo, cyano, C₁₋₆alkyl, C₂₋₆alkenyl, C₂₋₆alkynyl, C₃₋₈cycloalkyl, C₆₋₁₀aryl, 3- to 12-membered heterocycloalkyl, 5- to 10-membered heteroaryl, —C(═O)—C₀₋₃alkylene-C₃₋₈cycloalkyl, —C(═O)—C₀₋₃alkylene-C₆₋₁₀aryl, —C(═O)—C₀₋₃alkylene-3- to 12-membered heterocycloalkyl, —C(═O)—C₀₋₃alkylene-5- to 10-membered heteroaryl, —OR, —S(═O)ₘR^{k}, —P(═O)R^{kk}R^{mm}, —NR^{k}R^{m}, —C(═O)OR^{k}, or —C(═O)NR^{k}R^{m},
each X² is independently selected from the group consisting of halo, cyano, oxo, C₀₋₃alkylene-ORⁿ, C₀₋₃alkylene-S(═O)ₘRⁿ, C₀₋₃alkylene-NRⁿR^{o}, C₀₋₃alkylene-C(═O)NRⁿR^{o}, C₀₋₃alkylene-C(═O)ORⁿ, and R^{S3}, in which R^{S3} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, Ca-alkylene-3- to 12-membered heterocycloalkyl, or C₀₋₃alkylene-5- to 10-membered heteroaryl;
and R^{S3} is optionally substituted with one or more substituents independently selected from the group consisting of halo, oxo, cyano, C₀₋₃alkylene-OR^{p}, C₀₋₃alkylene-S(═O)ₘR^{p}, C₀₋₃alkylene-NR^{p}R^{q}, C₀₋₃alkylene-C(═O)NR^{p}R^{q}, C₀₋₃alkylene-C₀₋₃alkylene-C(═O)OR^{p}, C₁₋₆alkyl, C₂₋₆alkenyl, C₂₋₆alkynyl, C₁₋₆haloalkyl, and R^{S4}, in which R^{S4} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;
and each R^{S4} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w4}, and —NR^{w4}R^{x4};
R³ is halo, C₁₋₃alkyl, C₁₋₃haloalkyl, C₂₋₃alkenyl, C₂₋₃alkynyl, C₃₋₆cycloalkyl, —CN, —OR^{r}, —C(═O)R^{r}, —S(═O)ₘR^{r}, NR^{r}R^{t}, or —C(═O)OR^{r}, wherein C₁₋₃alkyl, C₂₋₃alkenyl and C₂₋₃alkynyl are optionally substituted with one C₃₋₆cycloalkyl;
R⁴ is C₁₋₆alkyl, C₁₋₆haloalkyl, S(═O)ₘR^{u}, C₀₋₃alkylene-C₃₋₈cycloalkyl, C₀₋₃alkylene-C₆₋₁₀ aryl, C₀₋₃alkylene-3- to 12-membered heterocycloalkyl, or C₀₋₃alkylene-5- to 10-membered heteroaryl, wherein C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl are optionally substituted with one or more substituents independently selected from the group consisting of halo, oxo, C₁₋₆alkyl, C₂₋₆alkenyl, C₂₋₆alkynyl, cyano, C₁₋₆haloalkyl, OR^{w5}, and NR^{w5}R^{x5};
each of R^{a} and R^{b}, independently, is H or R^{S5}, in which R^{S5} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;
and R^{S5} is optionally substituted with one or more substituents independently selected from the group consisting of halo, cyano, oxo, C₁₋₆haloalkyl, C₀₋₃alkylene-OR^{c2}, C₀₋₃alkylene-C(═O)R^{c2}, C₀₋₃alkylene-C(═O)OR^{c2}, C₀₋₃alkylene-OC(═O)R^{c2}, C₀₋₃alkylene-C(═O)NR^{c2}R^{d2}, C₀₋₃alkylene-S(═O)ₘR^{c2}, C₀₋₃alkylene-S(═O)₂NR^{c2}R^{d2}, C₀₋₃alkylene-NR^{c2}R^{d2}, C₀₋₃alkylene-NR^{c2}C(═O)R^{c2}, C₀₋₃alkylene-NR^{c2}C(═O)OR^{c2}, C₀₋₃alkylene-NR^{c2}C(═O)NR^{c2}R^{d2}, C₀₋₃alkylene-NR^{c2}S(═O)₂R^{c2}, C₀₋₃alkylene-C(═O)NR^{c2}S(═O)₂R^{c2}, C₀₋₃alkylene-NR^{c2}S(═O)₂NR^{c2}R^{d2}, C₀₋₃alkylene-N(S(═O)₂R^{c2})₂, and R^{S6}, in which R^{S6} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;
and each R^{S6} is optionally substituted with one or more substituents independently selected from the group consisting of halo, cyano, oxo, C₁₋₆alkyl, C₂₋₆alkenyl, C₂₋₆alkynyl, C₁₋₆haloalkyl, C₀₋₃alkylene-NR^{e2}R^{f2}, C₀₋₃alkylene-OR^{e2}, C₀₋₃alkylene-NR^{e2}C(═O)R^{e2}, C₀₋₃alkylene-NR^{e2}C(═O)OR^{e2}, C₀₋₃alkylene-NR^{e2}C(═O)NR^{e2}R^{f2}, C₀₋₃alkylene-OC(═O)R^{e2}, C₀₋₃alkylene-C(═O)OR^{e2}, C₀₋₃alkylene-C(═O)NR^{e2}R^{f2}, C₀₋₃alkylene-C(═O)R^{e2}, C₀₋₃alkylene-S(═O)ₘR^{e2}, C₀₋₃alkylene-S(═O)₂NR^{e2}R^{f2}, C₀₋₃alkylene-NR^{e2}S(═O)₂R^{e2}, C₀₋₃alkylene-C(═O)NR^{e2}S(═O)₂R^{e2}, C₀₋₃alkylene-NR^{e2}S(═O)₂NR^{e2}R^{f2}, and R^{S7}, in which R^{S7} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;
and each R^{S7} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w6}, and —NR^{w6}R^{x6};
each of R^{c}, R^{c2}, R^{d}, R^{d′}, and R^{d2}, independently, is H or R^{S8}, in which R^{S8} is C₁₋₆alkyl, C₂₋₆alkenyl, C₁₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;
and each R^{S8} is optionally substituted with one or more substituents independently selected from the group consisting of halo, cyano, oxo, C₁₋₆alkyl, C₂₋₆alkenyl, C₂₋₆alkynyl, C₁₋₆haloalkyl, C₀₋₃alkylene-NR^{e3}R^{f3}, C₀₋₃alkylene-OR^{e3}, C₀₋₃alkylene-C(═O)OR^{et}, C₀₋₃alkylene-C(═O)NR^{e3}R^{f3}, C₀₋₃alkylene-C(═O)R^{e3}, C₀₋₃alkylene-S(═O)ₘR^{e3}, C₀₋₃alkylene-S(═O)₂NR^{e3}R^{f3}, C₀₋₃alkylene-NR^{f3}C(═O)R^{e3}, C₀₋₃alkylene-NR^{f3}S(═O)ₘR^{e3}, and R^{S9}, in which R^{S9} is C₀₋₃alkylene-C₃₋₈cycloalkyl, C₀₋₃alkylene-C₆₋₁₀aryl, C₀₋₃alkylene-3- to 12-membered heterocycloalkyl, C₀₋₃alkylene-5- to 10-membered heteroaryl;
and each R^{S9} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w7}, and —NR^{w7}R^{x7};
each of R^{e}, R^{e2}, R^{e3}, R^{f}, R^{f2}, and R^{f3}, independently, is H or R^{S10}, in which R^{S10} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;
and each R^{S10} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w8}, and —NR^{w8}R^{x8};
each of R^{kk} and R^{mm}, is independently selected from the group consisting of R^{k}, —OR^{k}, and —NR^{k}R^{m};
each of R^{k} and R^{m}, independently, is H or R^{z}, in which R^{z} is C₁₋₄alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;
and each R^{z} is optionally substituted with one or more substituents independently selected from the group consisting of halo, cyano, oxo, C₁₋₆alkyl, C₂₋₆alkenyl, C₂₋₆alkynyl, C₁₋₆haloalkyl, C₀₋₃alkylene-NRⁿ²R^{o2}, C₀₋₃alkylene-ORⁿ², C₀₋₃alkylene-C(═O)ORⁿ², C₀₋₃alkylene-C(═O)NRⁿ²R^{o2}, C₀₋₃alkylene-C(═O)Rⁿ², C₀₋₃alkylene-S(═O)ₘRⁿ², C₀₋₃alkylene-S(═O)₂NRⁿ²R^{o2}, and R^{S11}, in which R^{S11} is C₀₋₃alkylene-C₃₋₈cycloalkyl, C₀₋₃alkylene-C₆₋₁₀aryl, C₀₋₃alkylene-3- to 12-membered heterocycloalkyl, C₀₋₃alkylene-5- to 10-membered heteroaryl;
and each R^{S11} is optionally substituted with one or more substituents independently selected from the group consisting of halo, oxo, cyano, C₀₋₃alkylene-OR^{p2}, C₀₋₃alkylene-S(═O)ₘR^{p2}, C₀₋₃alkylene-NR^{p2}R^{q2}, C₀₋₃alkylene-C(═O)NR^{p2}R^{q2}, C₀₋₃alkylene-C₀₋₃alkylene-C(═O)OR^{p2}, C₁₋₆alkyl, C₂₋₆alkenyl, C₂₋₆alkynyl, C₁₋₆haloalkyl, and R^{S12}, in which R^{S12} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;
each R^{S12} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w9}, and —NR^{w9}R^{x9};
each of Rⁿ, Rⁿ², R^{o}, and R^{o2}, independently, is H or R^{S13}, in which R^{S13} is C₁₋₆alkyl, C₂₋₆-alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;
each R^{S13} is optionally substituted with one or more substituents independently selected from the group consisting of halo, oxo, cyano, C₀₋₃alkylene-OR^{p3}, C₀₋₃alkylene-S(═O)ₘR^{p3}, C₀₋₃alkylene-NR^{p3}R^{q3}, C₀₋₃alkylene-C(═O)NR^{p3}R^{q3}, C₀₋₃alkylene-C(═O)OR^{p3}, C₁₋₆alkyl, C₂₋₆alkenyl, C₂₋₆alkynyl, C₁₋₆haloalkyl, and R^{S14}, in which R^{S14} is C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;
each R^{S14} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w10}, and —NR^{w10}R^{x10};
each of R^{p}, R^{p2}, R^{p3}, R^{q}, R^{q2}, and R^{q3}, independently, is H or R^{S15}, in which R^{S15} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;
each R^{S15} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —OR^{w11}, and —NR^{w11}R^{x11};
each of R^{r}, R^{t}, and R^{u}, independently, is H or R^{S16}, in which R^{S16} is C₁₋₆alkyl, C₂₋₆alkenyl, C₂₋₆alkynyl, C₀₋₃alkylene-C₃₋₈cycloalkyl, C₀₋₃alkylene-C₆₋₁₀aryl, C₀₋₃alkylene-3- to 12-membered heterocycloalkyl, or C₀₋₃alkylene-5- to 10-membered heteroaryl;
and each R^{S16} is optionally substituted with one or more substituents independently selected from the group consisting of halo, oxo, C₁₋₆alkyl, C₂₋₆alkenyl, C₂₋₆alkynyl, cyano, C₁₋₆haloalkyl, —C(═O)OR^{w12}, —OR^{w12}, and —NR^{w12}R^{x12};
each R^{w}, R^{w2}, R^{w3}, R^{w4}, R^{w5}, R^{w6}, R^{w7}, R^{w8}, R^{w9}, R^{w10}, R^{w11}, R^{w12}, R^{x}, R^{x2}, R^{x3}, R^{x4}, R^{x5}, R^{x6}, R^{x7}, R^{x8}, R^{x9}, R^{x10}, R^{x11}, and R^{x12}, independently, is H, C₁₋₆alkyl, C₂₋₆alkenyl, C₂₋₆alkynyl or C₁₋₆haloalkyl;
each of n and p independently is 0, 1, 2, 3, 4, or 5, wherein when T¹ is H, n is 0, and when T² is H, p is 0; and
m is 0, 1, or 2;
with the proviso that the compound is not

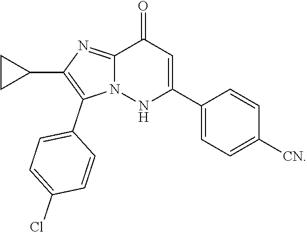 [SMILES: N#Cc1ccc(-c2cc(=O)c3nc(C4CC4)c(-c4ccc(Cl)cc4)n3[nH]2)cc1]

2. The compound of claim 1, wherein Q¹ is a bond and T¹ is C₆₋₁₀aryl, 3- to 12-membered heterocycloalkyl, or 5- to 10-membered heteroaryl, and n is 0, 1, 2, 3 or 4.

3. The compound of claim 1, wherein Q¹ is a bond or —CH₂— and T¹ is C₃₋₈-cycloalkyl, C₆₋₁₀aryl, 3- to 12-membered heterocycloalkyl, 5- to 10-membered heteroaryl, or —C(═O)NR^{a}R^{b}.

4. The compound of claim 1, wherein Q¹ is a bond or —CH₂—, T¹ is —C(═O)NR^{a}R^{b} and n is 0.

5. The compound of claim 1, wherein T¹ is 9- or 10-membered bicyclic heteroaryl.

6. The compound of claim 1, wherein one of R^{a} and R^{b} is H or methyl.

7. The compound of claim 1, wherein one of R^{a} and R^{b} independently is pyridinyl, pyrazinyl, pyridazinyl, pyrimidinyl, indolyl, indazolyl, benzimidazolyl, imidazopyridinyl, quinolinyl, isoquinolinyl, quinazolinyl, oxazolyl, isoxazolyl, thiazolyl, isothiazolyl, benzoxazolyl, oxadiazolyl, triazolyl, imidazolyl, furan, or thiophenyl, and the other is hydrogen or methyl.

8. The compound of claim 1, wherein R² is Q²-T²-(X²)ₚ, Q² is a bond, T² is H, halo, cyano, C₁₋₆alkyl, C₁₋₆haloalkyl, C₂₋₆alkenyl, or C₂₋₆alkynyl, and each X² independently is halo or —OC₁₋₆alkyl.

9. The compound of claim 1, wherein R² is H, cyano, methyl or methoxymethyl.

10. The compound of claim 1, wherein R³ is C₁₋₃alkyl, C₁₋₃haloalkyl, —CN, —S(═O)₂C₁₋₃alkyl or —C(═O)OC₀₋₃alkyl.

11. The compound of claim 1, wherein R³ is —CN, C₁₋₃alkyl, C₁₋₃haloalkyl or —C(═O)OC₁₋₃alkyl.

12. The compound of claim 1, wherein R³ is —CN or —CF₃.

13. The compound of claim 1, wherein R⁴ is C₁₋₃alkyl, C₁₋₆haloalkyl, —S(═O)₂C₁₋₃alkyl, C₃₋₈cycloalkyl, C₆₋₁₀aryl, 3- to 12-membered heterocycloalkyl, or 5- to 10-membered heteroaryl, wherein C₃₋₈cycloalkyl, C₆₋₁₀aryl, 3- to 12-membered heterocycloalkyl, or 5- to 10-membered heteroaryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}.

14. The compound of claim 1, wherein R⁴ is C₃₋₈cycloalkyl or C₆₋₁₀aryl, wherein C₃₋₈cycloalkyl and C₆₋₁₀aryl are optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

15. The compound of claim 1, wherein R⁴ is phenyl optionally substituted with 1-3 substituents selected from the group consisting of halo, oxo, C₁₋₆alkyl, C₂₋₆alkenyl, C₂₋₆alkynyl, cyano, C₁₋₆haloalkyl, —OR^{w5}, and —NR^{w5}R^{x5}, wherein R^{w5} and R^{x5} are independently H, C₁₋₆alkyl or C₁₋₆haloalkyl.

16. The compound of claim 1, wherein T¹ is aryl or heteroaryl.

17. The compound of claim 1, wherein T¹ is 5- or 6-membered monocyclic heteroaryl or 9- or 10-membered bicyclic heteroaryl.

18. The compound of claim 1, wherein T¹ is pyridinyl, pyrazinyl, pyridazinyl, pyrimidinyl, indolyl, indolinyl, isoindolyl, isoindolinyl, indazolyl, pyrazolopyridinyl, pyrazolopyrimidinyl, oxazolopyrimidinyl, oxazolopyridinyl, imidazopyridinyl, benzimidazolyl, tetrahydrobenzimidazolyl, benzofuranyl, dihydrobenzofuranyl, isobenzofuranyl, dihydroisobenzofuranyl, triazolopyridinyl, benzothiazolyl, azabenzimidazolyl, azabenzoxazolyl, azabenzothiazolyl, imidazopyridinyl, quinolinyl, isoquinolinyl, quinazolinyl, oxazolyl, isoxazolyl, thiazolyl, isothiazolyl, benzoxazolyl, benzodioxolyl, chromanyl, tetrahydrooxazoloazepinyl, tetrahydrobenzoxazolyl, oxadiazolyl, thiadiazolyl, pyrazolyl, triazolyl, imidazolyl, furanyl, or thiophenyl.

19. The compound of claim 1, wherein T¹ is 3- to 12-membered heterocycloalkyl.

20. The compound of claim 19, wherein T¹ is piperazine, piperidine, quinuclidine, or morpholine.

21. The compound of claim 1, wherein the compound is of Formula (IIa):

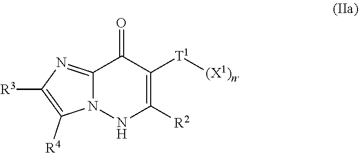

22. A compound of Formula (III): 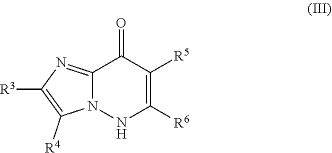
or a pharmaceutically acceptable salt, pharmaceutically acceptable solvate or pharmaceutically acceptable hydrate thereof, wherein
R⁵ is selected from the group consisting of —C(═O)NR⁹R¹⁰, phenyl optionally substituted with 1, 2, or 3 R¹¹, and a 5 to 10-membered heteroaryl optionally substituted with 1, 2, or 3 R¹²;
R⁶ is selected from the group consisting of —H, halo, —CN, C₁₋₃haloalkyl, and —C₁₋₃alkyl;
R⁷ is selected from the group consisting of —CN, —C₁₋₃alkyl, and C₀₋₃haloalkyl,
R⁸ is selected from the group consisting of phenyl optionally substituted with 1, 2, or 3 R¹³, and a 5- or 6-membered monocyclic heteroaryl optionally substituted with 1, 2, or 3 R¹⁴;
R⁹ and R¹⁰ are independently selected from the group consisting of —H, phenyl optionally substituted with 1, 2, or 3 R¹⁵, and a 5 to 10-membered heteroaryl optionally substituted with 1, 2, or 3 R¹⁶;
each R¹¹, R¹³ and R¹⁵ is independently selected from the group consisting of —C₁₋₃alkyl, —C₁₋₃haloalkyl, halo, —CN, —OH, —OC₁₋₃alkyl, and —OC₁₋₃haloalkyl; and
each R¹², R¹⁴ and R¹⁶ is independently selected from the group consisting of —C₁₋₃alkyl, —C₁₋₃haloalkyl, halo, —CN, —OH, —OC₁₋₃alkyl, and —OC₁₋₃haloalkyl.

23. The compound of claim 22, wherein R⁵ is selected from the group consisting of —C(═O)NR⁹R¹⁰, phenyl optionally substituted with 1, 2, or 3 R¹¹, a 5- or 6-membered monocyclic heteroaryl optionally substituted with 1, 2, or 3 R¹², and a 9- or 10-membered bicyclic heteroaryl optionally substituted with 1, 2, or 3 R¹²; and one of R⁹ and R¹⁰ is —H and the other of R⁹ and R¹⁰ is selected from the group consisting of phenyl optionally substituted with 1, 2, or 3 R¹⁵, a 5- or 6-membered monocyclic heteroaryl optionally substituted with 1, 2, or 3 R¹⁶, and a 9- or 10-membered bicyclic heteroaryl optionally substituted with 1, 2, or 3 R¹⁶.

24. The compound of claim 22, wherein R⁵ is selected from the group consisting of —C(═O)NR⁹R¹⁰, phenyl optionally substituted with 1, 2, or 3 R¹¹, a 5- or 6-membered monocyclic heteroaryl optionally substituted with 1, 2, or 3 R¹², and a 9- or 10-membered bicyclic heteroaryl optionally substituted with 1, 2, or 3 R¹²; one of R⁹ and R¹⁰ is —H and the other of R⁹ and R¹⁰ is selected from the group consisting of phenyl optionally substituted with 1, 2, or 3 R¹⁵, a 5- or 6-membered monocyclic heteroaryl optionally substituted with 1, 2, or 3 R¹⁶, and a 9- or 10-membered bicyclic heteroaryl optionally substituted with 1, 2, or 3 R¹⁶; R⁶ is C₁₋₃haloalkyl or —C₁₋₃alkyl; R⁷ is —CN or —CF₃; and R⁸ is phenyl optionally substituted with 1, 2, or 3 substituents independently selected from the group consisting of —C₁₋₃alkyl, —C₁₋₃haloalkyl, halo, —CN, —OC-s3alkyl, and —OC₁₋₃haloalkyl.

25. The compound of claim 1, or a pharmaceutically acceptable salt, pharmaceutically acceptable solvate or pharmaceutically acceptable hydrate thereof, wherein the compound is selected from the group consisting of

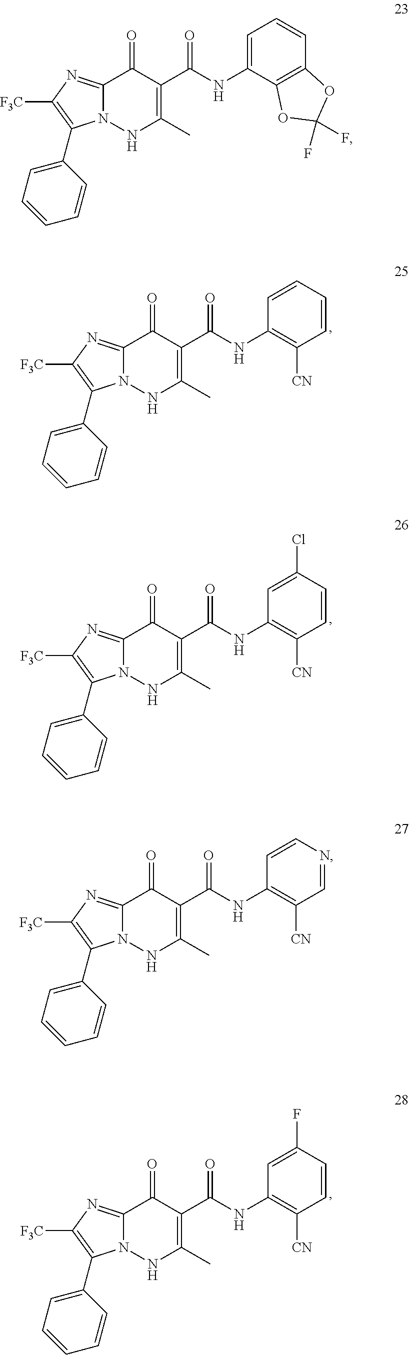 [SMILES: Cc1[nH]n2c(-c3ccccc3)c(C(F)(F)F)nc2c(=O)c1C(=O)Nc1cccc2c1OC(F)(F)O2 ; Cc1[nH]n2c(-c3ccccc3)c(C(F)(F)F)nc2c(=O)c1C(=O)Nc1ccccc1C#N ; Cc1[nH]n2c(-c3ccccc3)c(C(F)(F)F)nc2c(=O)c1C(=O)Nc1cc(Cl)ccc1C#N ; Cc1[nH]n2c(-c3ccccc3)c(C(F)(F)F)nc2c(=O)c1C(=O)Nc1ccncc1C#N ; Cc1[nH]n2c(-c3ccccc3)c(C(F)(F)F)nc2c(=O)c1C(=O)Nc1cc(F)ccc1C#N]

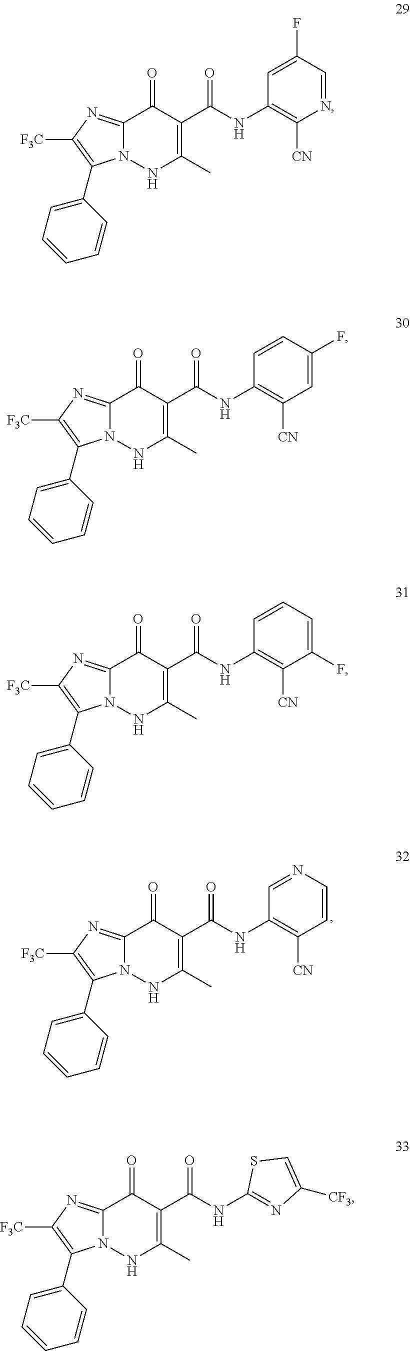 [SMILES: Cc1[nH]n2c(-c3ccccc3)c(C(F)(F)F)nc2c(=O)c1C(=O)Nc1cc(F)cnc1C#N ; Cc1[nH]n2c(-c3ccccc3)c(C(F)(F)F)nc2c(=O)c1C(=O)Nc1ccc(F)cc1C#N ; Cc1[nH]n2c(-c3ccccc3)c(C(F)(F)F)nc2c(=O)c1C(=O)Nc1cccc(F)c1C#N ; Cc1[nH]n2c(-c3ccccc3)c(C(F)(F)F)nc2c(=O)c1C(=O)Nc1cnccc1C#N ; Cc1[nH]n2c(-c3ccccc3)c(C(F)(F)F)nc2c(=O)c1C(=O)Nc1nc(C(F)(F)F)cs1]

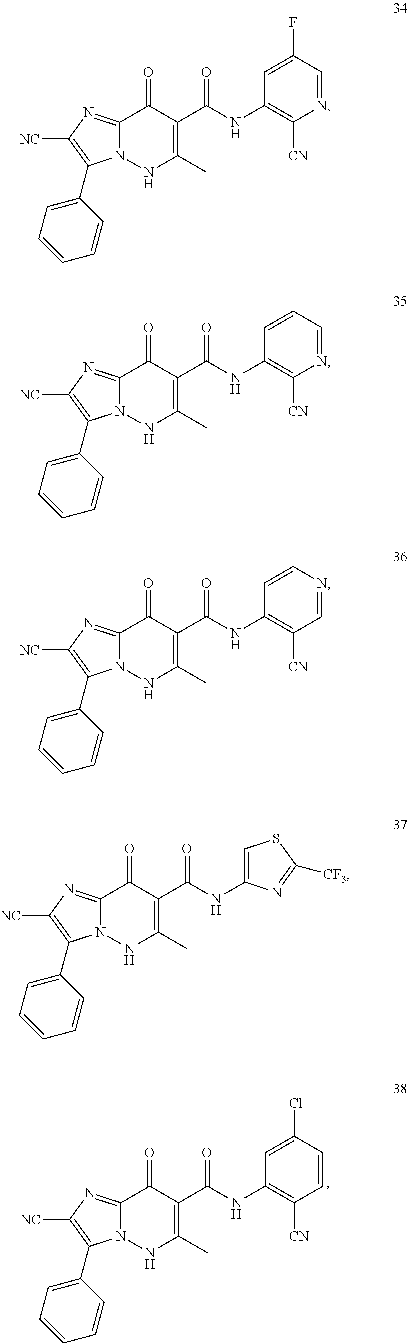 [SMILES: Cc1[nH]n2c(-c3ccccc3)c(C#N)nc2c(=O)c1C(=O)Nc1cc(F)cnc1C#N ; Cc1[nH]n2c(-c3ccccc3)c(C#N)nc2c(=O)c1C(=O)Nc1cccnc1C#N ; Cc1[nH]n2c(-c3ccccc3)c(C#N)nc2c(=O)c1C(=O)Nc1ccncc1C#N ; Cc1[nH]n2c(-c3ccccc3)c(C#N)nc2c(=O)c1C(=O)Nc1csc(C(F)(F)F)n1 ; Cc1[nH]n2c(-c3ccccc3)c(C#N)nc2c(=O)c1C(=O)Nc1cc(Cl)ccc1C#N]

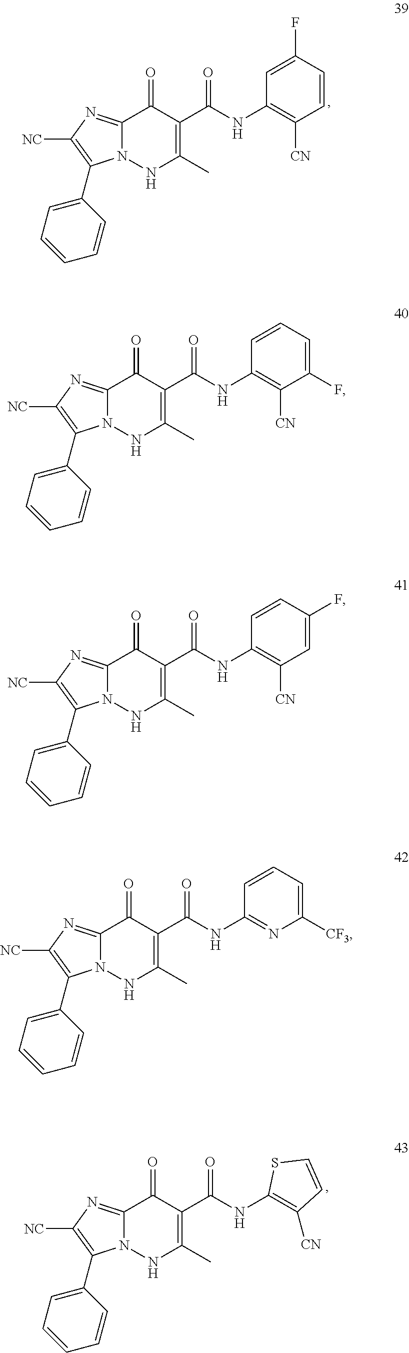 [SMILES: Cc1[nH]n2c(-c3ccccc3)c(C#N)nc2c(=O)c1C(=O)Nc1cc(F)ccc1C#N ; Cc1[nH]n2c(-c3ccccc3)c(C#N)nc2c(=O)c1C(=O)Nc1cccc(F)c1C#N ; Cc1[nH]n2c(-c3ccccc3)c(C#N)nc2c(=O)c1C(=O)Nc1ccc(F)cc1C#N ; Cc1[nH]n2c(-c3ccccc3)c(C#N)nc2c(=O)c1C(=O)Nc1cccc(C(F)(F)F)n1 ; Cc1[nH]n2c(-c3ccccc3)c(C#N)nc2c(=O)c1C(=O)Nc1sccc1C#N]

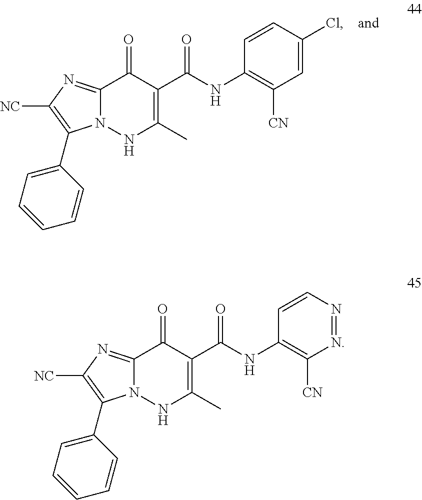 [SMILES: Cc1[nH]n2c(-c3ccccc3)c(C#N)nc2c(=O)c1C(=O)Nc1ccc(Cl)cc1C#N ; "and" ; Cc1[nH]n2c(-c3ccccc3)c(C#N)nc2c(=O)c1C(=O)Nc1ccnnc1C#N]

26. The compound of claim 1, or a pharmaceutically acceptable salt, pharmaceutically acceptable solvate or pharmaceutically acceptable hydrate thereof, wherein the compound is selected from the group consisting of

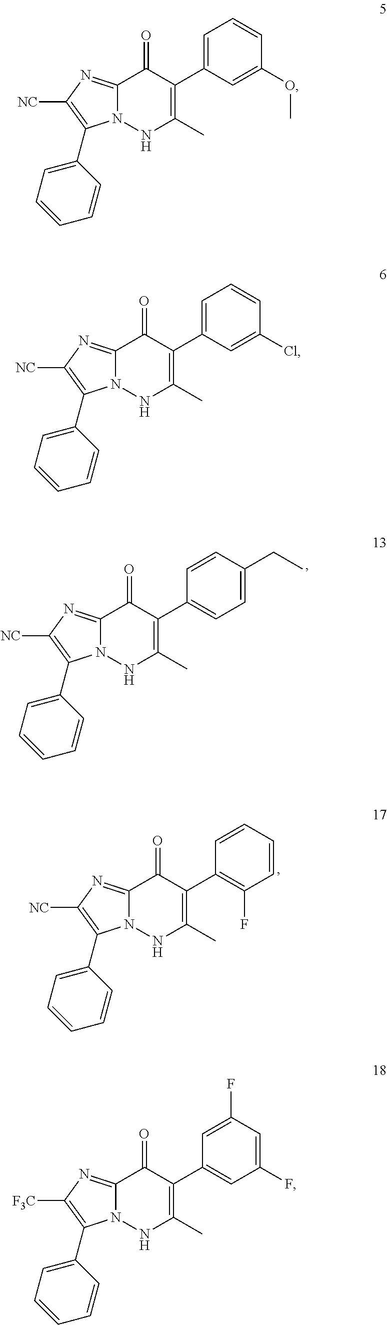 [SMILES: COc1cccc(-c2c(C)[nH]n3c(-c4ccccc4)c(C#N)nc3c2=O)c1 ; Cc1[nH]n2c(-c3ccccc3)c(C#N)nc2c(=O)c1-c1cccc(Cl)c1 ; CCc1ccc(-c2c(C)[nH]n3c(-c4ccccc4)c(C#N)nc3c2=O)cc1 ; Cc1[nH]n2c(-c3ccccc3)c(C#N)nc2c(=O)c1-c1ccccc1F ; Cc1[nH]n2c(-c3ccccc3)c(C(F)(F)F)nc2c(=O)c1-c1cc(F)cc(F)c1]

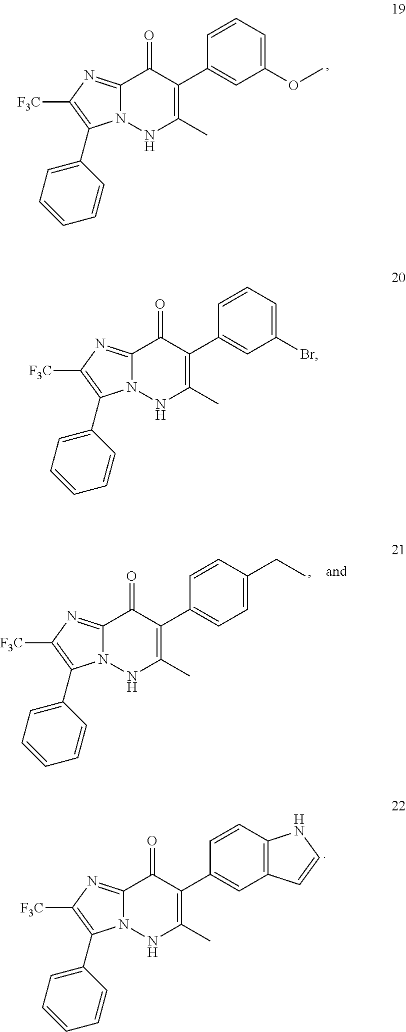 [SMILES: COc1cccc(-c2c(C)[nH]n3c(-c4ccccc4)c(C(F)(F)F)nc3c2=O)c1 ; Cc1[nH]n2c(-c3ccccc3)c(C(F)(F)F)nc2c(=O)c1-c1cccc(Br)c1 ; CCc1ccc(-c2c(C)[nH]n3c(-c4ccccc4)c(C(F)(F)F)nc3c2=O)cc1 ; Cc1[nH]n2c(-c3ccccc3)c(C(F)(F)F)nc2c(=O)c1-c1ccc2[nH]ccc2c1]

27. A pharmaceutical composition comprising the compound of claim 1, or a pharmaceutically acceptable salt, pharmaceutically acceptable solvate or pharmaceutically acceptable hydrate thereof, together with a pharmaceutically acceptable diluent or carrier.

28. A method of inhibiting the cGAS/STING pathway in a cell, comprising contacting the cell with the compound of claim 1, or a pharmaceutically acceptable salt, pharmaceutically acceptable solvate or pharmaceutically acceptable hydrate thereof.

29. A method of inhibiting cytokine production in a cell, comprising contacting the cell with the compound of claim 1, or pharmaceutically acceptable salt, pharmaceutically acceptable solvate or pharmaceutically acceptable hydrate thereof.

30. A method of treating a cGAS/STING pathway-mediated condition, comprising administering to a subject in need thereof an effective amount of a compound of claim 1, or a pharmaceutically acceptable salt, pharmaceutically acceptable solvate or pharmaceutically acceptable hydrate thereof.

31. The method of claim 30, wherein the cGAS/STING pathway-mediated condition is an autoimmune, inflammatory, or neurodegenerative condition.

32. A method of treating a disease in a subject, comprising administering to the subject in need thereof a therapeutically effective amount of the compound of claim 1, or a pharmaceutically acceptable salt, pharmaceutically acceptable solvate or pharmaceutically acceptable hydrate thereof, wherein the disease is selected from the group consisting of systemic inflammatory response syndrome (SIRS), sepsis, septic shock, atherosclerosis, celiac disease, dermatomyositis, scleroderma, interstitial cystitis, transplant rejection (e.g. graft-versus-host disease), Aicardi-Goutieres Syndrome, Hutchison Guilford progeria syndrome, Singleton-Merten Syndrome, proteasome-associated autoinflammatory syndrome, SAVI (STING-associated vasculopathy with onset in infancy), CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenic purpura, thrombotic thrombocytopenic purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjogren's syndrome, X-linked reticulate pigmentary disorder, polymyositis, spondyloenchondrodysplasia, age-related macular degeneration, Alzheimer's disease and Parkinson's disease.

33. A method of treating a disease in a subject, comprising administering to the subject in need thereof a therapeutically effective amount of the compound of claim 1, including any pharmaceutically acceptable salt, pharmaceutically acceptable solvate or pharmaceutically acceptable hydrate thereof, in combination with a Janus Kinase (Jak) inhibitor, wherein the disease is selected from the group consisting of systemic inflammatory response syndrome (SIRS), sepsis, septic shock, atherosclerosis, celiac disease, dermatomyositis, scleroderma, interstitial cystitis, transplant rejection (e.g. graft-versus-host disease), Aicardi-Goutieres Syndrome, Hutchison Guilford progeria syndrome, Singleton-Merten Syndrome, proteasome-associated autoinflammatory syndrome, SAVI (STING-associated vasculopathy with onset in infancy), CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenic purpura, thrombotic thrombocytopenic purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjogren's syndrome, X-linked reticulate pigmentary disorder, polymyositis, spondyloenchondrodysplasia, age-related macular degeneration, Alzheimer's disease and Parkinson's disease.

34. A compound of claim 1, for use in the treatment of a cGAS/STING pathway-mediated condition.

35. A compound of claim 1, for use in combination with a Janus Kinase inhibitor, for the treatment of a disease selected from the group consisting of systemic inflammatory response syndrome (SIRS), sepsis, septic shock, atherosclerosis, celiac disease, dermatomyositis, scleroderma, interstitial cystitis, transplant rejection (e.g. graft-versus-host disease), Aicardi-Goutieres Syndrome, Hutchison Guilford progeria syndrome, Singleton-Merten Syndrome, proteasome-associated autoinflammatory syndrome, SAVI (STING-associated vasculopathy with onset in infancy), CANDLE (Chronic Atypical Neutrophilic Dermatosis with Lipodystrophy and Elevated Temperature) syndrome, chilblain lupus erythematosus, systemic lupus erythematosus, rheumatoid arthritis, juvenile rheumatoid arthritis, Wegener's disease, inflammatory bowel disease (e.g. ulcerative colitis, Crohn's disease), idiopathic thrombocytopenic purpura, thrombotic thrombocytopenic purpura, autoimmune thrombocytopenia, multiple sclerosis, psoriasis, IgA nephropathy, IgM polyneuropathies, glomerulonephritis, autoimmune myocarditis, myasthenia gravis, vasculitis, Type 1 diabetes, Type 2 diabetes, Sjogren's syndrome, X-linked reticulate pigmentary disorder, polymyositis, spondyloenchondrodysplasia, age-related macular degeneration, Alzheimer's disease and Parkinson's disease.

36. Composition comprising a compound of claim 1 and a Janus Kinase inhibitor.

37. A kit comprising a compound of claim 1 and a Janus Kinase inhibitor.


SEQUENCE LISTING (3 sequences)

SEQ ID NO: 1 - protein, 366 aa, Artificial Sequence
  Synthetic Construct
DAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 2 - DNA, 45 bp, Artificial Sequence
  Synthetic construct
tacagatctactagtgatctatgactgatctgtacatgatctaca

SEQ ID NO: 3 - DNA, 45 bp, Artificial Sequence
  Synthetic Construct
tgtagatcatgtacagatcagtcatagatcactagtagatctgta